(19) United States
(12) Patent Application Publication
(10) Pub. No.: US 2021/0398684 A1
(43) Pub. Date: Dec. 23, 2021

(54) SYSTEMS AND METHODS FOR AUTOMATED CONTACT TRACING

(71) Applicant: PricewaterhouseCoopers LLP, New York, NY (US)

(72) Inventors: Robert MESIROW, Falls Church, VA (US); Patrick PARODI, Bethesda, MD (US); Marc MAZZIE, Washington, DC (US); Stillman BRADISH, Washington, DC (US); Srdjan MARINOVIC, Washington, DC (US)

(73) Assignee: PricewaterhouseCoopers LLP, New York, NY (US)

(21) Appl. No.: 17/350,612

(22) Filed: Jun. 17, 2021

Related U.S. Application Data
(60) Provisional application No. 63/069,226, filed on Aug. 24, 2020.
(60) Provisional application No. 63/041,646, filed on Jun. 19, 2020.
(60) Provisional application No. 63/041,656, filed on Jun. 19, 2020.
(60) Provisional application No. 63/041,662, filed on Jun. 19, 2020.

(51) Int. Cl. G16H 50/30; H04W 4/02; G06Q 10/10; G16H 50/70; G06F 16/28

(52) U.S. Cl. CPC G16H 50/30; H04W 4/023; G16H 50/80; G16H 50/70; G06F 16/288; G06Q 10/105

(57) ABSTRACT

A system and method for generating a risk score for an observer device based on the wireless signals that are emitted from one or more wireless devices is provided. The system and method includes receiving, by an analytic server, a request for tracing data associated with a first device identifier of a first observer device; generating, by the analytic server responsive to receiving the request, tracing data including a second identifier of a second observer device and a risk score indicative of a degree of association between the first observer device and the second observer device; and transmitting, by the analytic server, the tracing data to a human resource (HR) server associated with an organization, wherein the tracing data causes the HR server to notify the second observer device.

### CROSS-REFERENCE TO RELATED APPLICATIONS

This application claims the benefit of U.S. Provisional Application No. 63/041,646, filed Jun. 19, 2020, U.S. Provisional Application No. 63/041,656, filed Jun. 19, 2020, U.S. Provisional Application No. 63/041,662, filed Jun. 19, 2020, and U.S. Provisional Application No. 63/069,226, filed Aug. 24, 2020, the entire contents of each of which are incorporated herein by reference.

### TECHNICAL FIELD

This application relates generally to systems and methods for automated contact tracing using electronic devices, such as wireless devices and other devices enabled with wireless transmitters.

### BACKGROUND

From an epidemiological perspective, granularity of the interactions, both in time and space, can be very instructive as to how a disease spreads through a population and for later tracing efforts. But where epidemiological study gathers and analyzes data to understand and predict the current and future spread of a disease, tracing is an investigatory effort to retrace specific interactions of particular individuals. Thus comparatively higher levels of granularity may suffice for certain epidemiological purposes, whereas tracing is premised on and expects very personalized or individualized information. With recent health emergencies spreading there has been an emerging need for tools to help expedite and automate tracing the historic interactions between individuals.

Modern societal norms create an expectation that people will carry some form of electronic device with them when they go to the office. These can be a great tool for gathering and referencing tracing data when there is a need to back-trace who came in contact with an infected person. It is well understood that data transmitted and received from personal devices, such as cell phones, can be captured and analyzed for various purposes, such as commercial marketing intelligence or law enforcement. The data captured from devices might allow for tracking and cataloging the actions of a device through the day could be analyzed for tracing purposes. Other technological approaches using personal devices for tracking data and analyzing user behavior, however, have issues of privacy and information quality. Privacy issues can come in many forms, including concerns for the confidentiality of health information and personal location tracking. Often the need for granular, personalized data-gathering is in tension with privacy concerns. Prior approaches have traded-off detailed, granular information for privacy, or vice-versa. What is therefore needed is a means for maintaining user privacy while benefitting from the detailed device data in tracing.

### SUMMARY

Described herein are systems and methods that can leverage the data generate from wireless devices to determine and identify contacts between people for automated contact tracing, while maintaining personal privacy. Embodiments herein describe systems and methods for locating and/or tracing devices using proximal groups of devices where device transmit observations of their interactions with one another but only within a geo-fenced location. The observation data can be used to identify whether individuals were in contact with one another and the extent of that contact. The data being exchanged, however, is a string for identifying the observer device, rather than the user, so personal tracking information is not transmitted openly. Nor is identifiable human information used for tracing analytics or stored together with the observed data from user devices, so for practical purposes the data captured from a wireless device is not readily associated with the user. The tracing data capture from a device is only associated with a person when only authorized individuals execute tracing processes that query the separate databases storing the device tracing data and the device-to-human associations.

Software applications for capturing tracing data may be downloaded and installed from public datastores (e.g., Apple App Store®, Google Play®), a dedicated public datastore (e.g., enterprise-specific App Store®, enterprise-specific Google Play®), or a private datastore (e.g., enterprise application server). An application may be installed onto a user mobile device that instructs the user device hardware to capture and exchange data with the tracing system. In some implementations, a user device may transmit and receive observation data with other user devices such that each user device captures observation data. The user device may then transmit observation data to the tracing system. And in some implementations, the user device may be connected to a separate hardware component that communicates with other devices for capturing observation data. In some cases, the separate device may be configured to transmit data to other devices that is received by those other devices as observation data, while the user device receives observation data from other devices. The user device then transmits the observation data to the tracing system.

In an embodiment, a system comprising a plurality of observer devices configured to generate observation data based on a plurality of wireless signals emitted from each of the plurality of observer devices, wherein the observer devices includes at least one mobile device paired with at least one tracer device respectively, and wherein the observer devices are configured to generate the observation data based on the wireless signals emitted from each of the at least one tracer device; a mobile device paired with a tracer device, the mobile device configured to receive the wireless signals emitted from the observer devices and transmit the observation data to an analytic server via one or more networks, and the tracer device configured to emit wireless signals containing the observation data for the mobile device paired with the tracer device; an analytic server configured to: receive the observation data from each observer device; identify a proximity relationship between the mobile device and a set of one or more observer devices based up on the observation data received for the mobile device and the set of one or more observer devices; and generate a risk score for the mobile device based upon the proximity relationship, the risk score indicative of a degree of association between the mobile device each observer device of the set of observer devices.

In one aspect, a method can generate risk scores for client devices based on wireless signals. The method includes receiving, by an analytic server, a request for tracing data associated with a first identifier of a first observer device; generating, by the analytic server responsive to receiving the request, tracing data including a second identifier of a second observer device and a risk score indicative of a degree of association between the first observer device and the second observer device, wherein the degree of association corresponds to at least one of a signal strength of the second observer device, a duration during which the first observer device detects an output from the second observer device having a signal strength at or above a predetermined value, a count of the number of times in which the first observer device detects the output from the second observer device; and transmitting, by the analytic server, the tracing data to a human resource (HR) associated with an organization, wherein the tracing data causes the HR server to notify the second observer device using the second identifier.

In another aspect, a system can generate risk scores for client devices based on wireless signals. The system includes an analytic server configured to: receive observation data from an observer device, wherein the observer device generates the observation data based on a plurality of wireless signals emitted from a plurality of observer devices; maintain, in a database and based on the observation data, an association between a plurality of observer identifiers of the plurality of observer devices and a plurality of signal strengths determined by the observer device, each signal strength of the plurality of signal strengths corresponding to a respective wireless signal of the plurality of wireless signals that is emitted by a respective observer device of the plurality of observer devices; and generate, by the analytic server and based on the database, a risk score indicative of a degree of association between a first observer device and a second observer device.

In another aspect, a method can generate risk scores for client devices based on wireless signals. The method includes capturing, by an application executing on a first observer device and responsive to entering a geofence location according to instructions from a human resource (HR) server, a wireless signal emitted from a second observer device; generating, by the application executing on the first observer device, observation data based on the wireless signal, wherein the observation data indicates a signal strength of the captured wireless signal; and transmitting, by the application executing on the observer device, the observation data to an analytic server causing the analytic server to determine a risk score that indicates a degree of proximity between the first observer device and the second observer device based on the observation data.

In another aspect, a system can generate risk scores for client devices based on wireless signals. The system includes a first observer device configured to, responsive to entering a geofence location according to instructions from a human resource (HR) server: capture, by an application executing on the first observer device, a wireless signal emitted from a second observer device; generate, by the application executing on the first observer device, observation data based on the wireless signal, wherein the observation data indicates a signal strength of the captured wireless signal; transmit, by the application executing on the observer device, the observation data to an analytic server causing the analytic server to determine a risk score that indicates a degree of proximity between the first observer device and the second observer device based on the observation data.

In another aspect, a system can generate risk scores for client devices based on wireless signals. The system includes an observation database configured to store user identifiers associated with observation device identifiers; and a human resource (HR) server configured to: transmit, to an analytic server, a request for tracing data associated with a first observer device; receive, from the analytic server, the tracing data, wherein the tracing data includes a risk score indicative of a degree of association between the first observer device and a second observer device; determine that the risk score exceeds a predetermined threshold; and transmit, to the second observer device and responsive to determining that the risk score exceeds the predetermined threshold, a message indicating a health status of a user of the second observer device.

In one aspect, a method can generate a risk level data indicating a risk level of a target device associated with a user. In some embodiments, the method includes selecting, by a server, the target device associated with the user. In some embodiments, the method includes obtaining, by the server, detection data of the target device. In some embodiments, the method includes determining, by the server, a hypercluster associated the target device. In some embodiments, the method includes determining, by the server, a pattern of movements of the target device. In some embodiments, the method includes generating, by the server, the risk level data indicating the risk level of the target device, according to the detection data, the hypercluster, and the pattern.

In some embodiments, generating the risk level data includes determining, by the server, a first risk score of the target device according to the detection data. In some embodiments, generating the risk level data includes determining, by the server, a second risk score of the hypercluster associated with the target device. In some embodiments, generating the risk level data includes determining, by the server, a third risk score of the target device according to the pattern of movements of the target device. In some embodiments, generating the risk level data includes determining an aggregated risk score, according to the first risk score, the second risk score, and the third risk score. In some embodiments, generating the risk level data includes determining the risk level of the target device according to the aggregated risk score.

In some embodiments, determining, by the server, the first risk score of the target device includes comparing, by the server, a number of detections of the target device by a plurality of observer devices against predetermined threshold values. In some embodiments, determining, by the server, the first risk score of the target device includes determining the first risk score, according to the comparison.

In some embodiments, determining, by the server, the second risk score of the hypercluster associated with the target device includes aggregating, by the server, risk scores of a plurality of devices associated with the hypercluster. In some embodiments, determining, by the server, the second risk score of the hypercluster associated with the target device includes determining, by the server, the second risk score of the hypercluster, according to the aggregated risk scores.

In some embodiments, determining, by the server, the third risk score of the target device according to the pattern of movements of the target device includes determining, by the server, the pattern of movements of the target device. In some embodiments, determining, by the server, the third risk score of the target device according to the pattern of movements of the target device includes predicting, by the server, coexistences of the target device with one or more devices, according to the pattern of movement.

In one aspect, a method can generate a risk level data indicating a risk level of a target device associated with a user. In some embodiments, the method includes selecting, by a server, the target device associated with the user. In some embodiments, the method includes determining, by the server, a first hypercluster associated with the target device. In some embodiments, the method includes determining, by the server, a coexistence of the hypercluster with a second hypercluster. In some embodiments, the method includes generating, by the server, the risk level data indicating the risk level of the target device according to the determined coexistence.

In some embodiments, generating, by the server, the risk level data indicating the risk level of the target device according to the determined coexistence includes determining, by the server, coexistences of one or more devices of the first hypercluster with one or more devices of the second hypercluster. In some embodiments, generating, by the server, the risk level data indicating the risk level of the target device according to the determined coexistence includes determining, by the server, a risk level of the first hypercluster, according to determined coexistences.

In one aspect, a method can generate a risk level data indicating a risk level of a target device associated with a user. In some embodiments, the method includes selecting, by a server, the target device associated with the user. In some embodiments, the method includes determining, by the server, time points of coexistences by the target device with one or more hyperclusters. In some embodiments, the method includes generating, by the server, the risk level data indicating the risk level of the target device according to the time points of coexistences.

In some embodiments, generating, by the server, the risk level data indicating the risk level of the target device according to the time points of coexistences includes determining, by the server, a first coexistence of the target device with one or more first devices associated with a first hypercluster at a first time point. In some embodiments, generating, by the server, the risk level data indicating the risk level of the target device according to the time points of coexistences includes determining, by the server, a second coexistence of the target device with one or more second devices associated with a second hypercluster at a second time point. In some embodiments, generating, by the server, the risk level data indicating the risk level of the target device according to the time points of coexistences includes determining, by the server, the risk level of the target device according to the first coexistence with the one or more first devices at the first time point and the second coexistence with the one or more second devices at the second device.

In one aspect, a method can generate a risk level data indicating a risk level of a target device associated with a user. In some embodiments, the method includes selecting, by a server, the target device associated with the user. In some embodiments, the method includes receiving, by the server from a plurality of devices, detection data of the target device. In some embodiments, the method includes generating, by the server, a profile of the target device based on the detection data from the plurality of devices. In some embodiments, the method includes generating, by the server, the risk level data indicating the risk level of the target device according to the profile of the target device.

In some embodiments, generating, by the server, the profile of the target device based on the detection data from the plurality of devices includes generating, by the server, a trajectory of locations of the target device. In some embodiments, the target device is a beacon device transmitting a wireless signal. In some embodiments, the plurality of devices generate the detection data, in response to detecting the wireless signal from the beacon device. In some embodiments, the method further includes generating, by the server for another device of the plurality of devices, another risk level data indicating another risk level of the another device according to the risk level of the target device.

In some embodiments, a method comprising receiving, by a server, observation data of a plurality of wireless signals observed by a plurality of observer devices, the observation data comprising spatial proximity and temporal persistence data between the plurality of observer devices; determining, by the server, that an observer device of the plurality of observer devices was proximate to a proximal grouping of electronic devices at a time point based on observation data associated with the observer device; identifying, by the server in a database, a label associated with the proximal grouping, the label associated with a weight; determining, by the server, a risk score for the observer device based on a set of one or more risk factors of the observation data and the weight associated with the proximal group identified by the label; and transmitting, by the server, a signal comprising the risk score to an administrator client device.

In some embodiments, a method comprising receiving, by a server, observation data of a plurality of wireless signals observed by a plurality of observer devices, the observation data comprising spatial proximity and temporal persistence data between the plurality of observer devices; receiving, by the server, a request to determine a risk score for an observer device for a time period; identifying, by the server, a proximal grouping of electronic devices with which the observer device has a proximate association during the time period based on the observation data; identifying, by the server, a label associated with the identified proximal grouping of electronic devices and a weight associated with the label; determining, by the server, a number of proximate associations between the proximal grouping of electronic devices and the plurality of observer devices within the time period; adjusting, by the server, the weight associated with the label based on the determined number of proximate associations; determining, by the server, a risk score for the observer device based on at least the adjusted weight; and transmitting, by the server, a signal comprising the risk score to a client device.

In some embodiments, a method comprising receiving, by a server, observation data of a plurality of wireless signals observed by a plurality of observer devices, the observation data comprising spatial proximity and temporal persistence data between the plurality of observer devices; determining, by the server, a pattern of an observer device, the pattern indicating an expected location of the observer device at the time point; receiving, by the server, a request to determine a risk score for an observer device for a time period; identifying, by the server, a conflict between the expected location of the pattern and an observed location of the observer device indicated in the observation data at a given time point; responsive to identifying the conflict: determining, by the server, a risk score for the observer device based on at least a second weight associated with the expected location at the time point; and transmitting, by the server to a client device, a signal indicating a risk tier for the observer device within the time period.

In some embodiments, a system and method for generating a risk score for an observer device based on the wireless signals that are emitted from one or more wireless devices is provided. The system and method includes receiving, by an analytic server, a request for tracing data associated with a first device identifier of a first observer device; generating, by the analytic server responsive to receiving the request, tracing data including a second identifier of a second observer device and a risk score indicative of a degree of association between the first observer device and the second observer device; and transmitting, by the analytic server, the tracing data to a human resource (HR) server associated with an organization, wherein the tracing data causes the HR server to notify the second observer device.

A system and method for generating risk scores for observer devices based on signals emitted from wireless observer devices is provided. Embodiments include receiving, by a server, a request for tracing data associated with a first device identifier of a first observer device; generating, by the server responsive to receiving the request, tracing data including a second identifier of a second observer device and a risk score indicative of a degree of association between the first observer device and the second observer device; and transmitting, by the server, the tracing data to a server associated with an organization, wherein the tracing data causes the server to notify the second observer device. Embodiments can include a mobile device paired with a tracer device emitting signals observer devices capture to generate observation data having information about the mobile device. The mobile device generates observation data using wireless signals the mobile devices captures.

It is to be understood that both the foregoing general description and the following detailed description are exemplary and explanatory and are intended to provide further explanation of the invention as claimed.

### BRIEF DESCRIPTION OF THE DRAWINGS

The accompanying drawings constitute a part of this specification and illustrate embodiments of the subject matter disclosed herein.

FIGS. 1A-1B illustrate network environments which may be useful for practicing embodiments described herein, according to an embodiment.

FIG. 1C illustrates a network environment for locating and/or tracing electronic devices, according to an embodiment.

FIG. 2 illustrates a flowchart for tracing and locating electronic devices, according to an embodiment.

FIG. 3 illustrates an example of system architecture for working with a third party to locate electronic devices, according to an embodiment.

FIG. 4 illustrates a first example of data flows for locating electronic devices, according to an embodiment.

FIG. 5 illustrates a second example of data flows for locating electronic devices, according to an embodiment.

FIG. 6 is an example network environment for generating risk scores based on wireless signals, according to an embodiment.

FIG. 7A is a block diagram depicting an example observer device of the network environment in FIG. 6, according to an embodiment.

FIG. 7B is a block diagram depicting an example analytic server of the network environment in FIG. 6, according to an embodiment.

FIG. 7C is a block diagram depicting an example HR server of the network environment in FIG. 6, according to an embodiment.

FIG. 8 is a flow diagram depicting a method for generating risk scores based on wireless signals, according to an embodiment.

FIG. 9 illustrates a flowchart for determining a risk level of a target device based on detection data of the target device, a hypercluster associated with the target device, and a pattern of movements of the target device, according to an embodiment.

FIG. 10 illustrates a flowchart for determining a risk level of a target device based on coexistences of the target device with one or more hyperclusters, according to an embodiment.

FIG. 11 illustrates a flowchart for determining a risk level of a target device based on time points of coexistences of target device with one or more hyperclusters, according to an embodiment.

FIG. 12 illustrates a flowchart for determining a risk level of a virtual observer device, according to an embodiment.

FIG. 13 shows a flow diagram of a method of generating a risk score based on electronic labels, according to an illustrative embodiment.

FIG. 14 shows a flow diagram of a method of generating a risk score based on sets of contextualized data, according to an illustrative embodiment.

FIG. 15 shows a flow diagram of a method for determining a risk score based on predictive data, according to an illustrative embodiment.

FIG. 16 shows components of a system, according an embodiment.

FIG. 17 shows execution steps of a method for contact tracing using a system with tracer devices, according to an embodiment.

### DETAILED DESCRIPTION

Reference will now be made to the illustrative embodiments illustrated in the drawings, and specific language will be used here to describe the same. It will nevertheless be understood that no limitation of the scope of the claims or this disclosure is thereby intended. Alterations and further modifications of the inventive features illustrated herein, and additional applications of the principles of the subject matter illustrated herein, which would occur to one ordinarily skilled in the relevant art and having possession of this disclosure, are to be considered within the scope of the subject matter disclosed herein. The present disclosure is here described in detail with reference to embodiments illustrated in the drawings, which form a part here. Other embodiments may be used and/or other changes may be made without departing from the spirit or scope of the present disclosure. The illustrative embodiments described in the detailed description are not meant to be limiting of the subject matter presented here.

As would be understood by persons of ordinary skill in the art, any number of the features described in any number of the embodiments below may be combined into a single embodiment, and the description of any features in separate embodiments is not intended to be limiting.

In general, as described in the below passages, an analytic server may monitor wireless signals detected by a plurality of electronic devices and, in some embodiments, generate data structures representing hyperclusters (also sometimes referred to as, “signal clusters” or “proximal groupings” of devices) by analyzing the wireless signals and detected observation received in the wireless signals. The analytic server and system database may receive and store observed data, which includes the interactions of an electronic device with other devices in an environment and/or one or more hyperclusters comprising other devices. When the analytic server receives a request from an enterprise server (e.g., human resources administrator server) or client computing devices (e.g., administrator computer) to trace the interactions of a particular target device, the analytic server may identify other electronic devices in the ecosystem that have interacted with the target device, based on the observation data for the target device and other electronic devices. More specifically, the analytic server may receive the observation data from the observer devices; each of the observer devices receive and report (or observe) observation data based on the wireless signals broadcasted by the target device and by the target device itself.

The analytic server may determine a risk score for infection based on, for example, values in the observation data, a signal context (e.g., associated hypercluster, label data), identified patterns of behavior, weighted values for comparatively busy or empty spaces, and a geolocation (e.g., office location) of the target device as indicated in the observations from the observer devices. In addition, the analytic server may perform a resolution of the wireless signals within the hypercluster to assign semantic meanings to the wireless signals. The semantic meaning may provide useful information on the location or environment that the target device was at some time point located in, such as location, business, and any other knowledge, which may weight or adjust a risk score in order refine the quality, accuracy, and precision of the risk score.

An administrative user of an enterprise network may enter a tracing request into a client computing device to tracing the interactions between devices of the system and the target device. The tracing request may trigger a client computing device of the enterprise network to query an enterprise administration database that stores correspondences between observer device identifiers and user employee identifiers or information. The tracing request may indicate an employee identifier and a given time frame; the query returns the corresponding observer device identifier. This observer identifier is for the target observer device, and is submitted as a query to the analytic server. The analytic server identifies in an analytics database, each of the observer identifiers that were in contact with the target observer device and generates risk scores for each of the observer devices and/or the target observer device. The results of these calculations may be presented to the administrator via a GUI on the client computing device. The analytic server does not have access to the administrator database, so the analytic server is unable to access and return employee identifiers. Rather, the analytic server returns the outputted analytics data and observer identifiers. The client computing device or server may query the administrator database to identify and resolve which user employees correspond to the observer identifiers mentioned in the output from the analytics data.

### I. Tracing Devices Using Proximal Groups of Devices

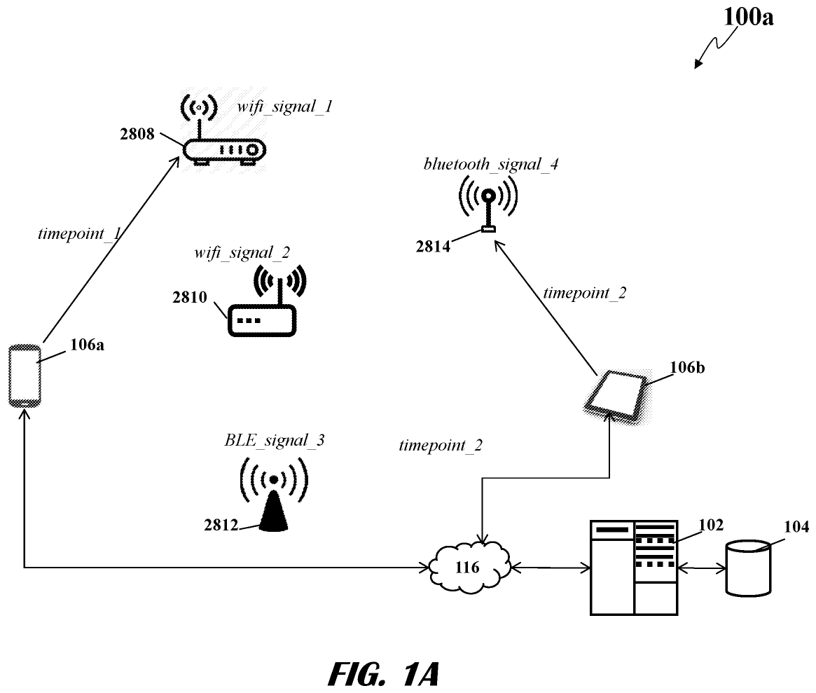

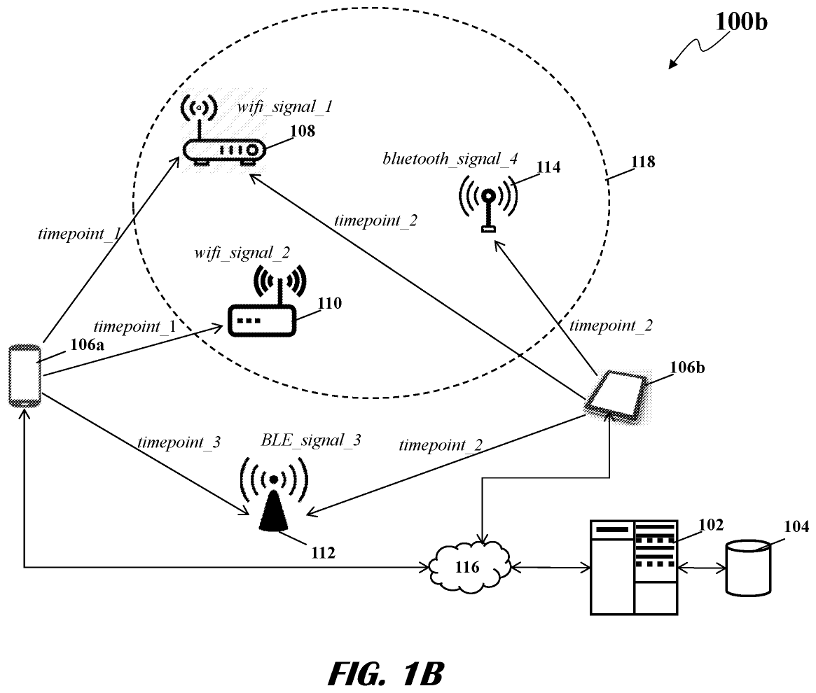

Embodiments herein describe systems and methods for locating and/or tracing interactions between devices using proximal groups of devices. FIG. 1A illustrates a network environment 100 which may be useful for practicing embodiments described herein, according to an embodiment. The network environment 100 may include an analytic server 102 and a database 104 coupled to the analytic server 102. The network environment 100 may include observer devices 106a, 106b (collectively referred to as, “observer devices 106”) that are interconnected with analytic server 102 via a network 116. The network environment 100 may include a Wi-Fi router 108, a Wi-Fi router 110, a BLE transmitter 112, and/or a Bluetooth transmitter 114. Although FIG. 1A shows only a select number of computing devices (e.g., observer devices, Wi-Fi routers, BLE transmitters, Bluetooth transmitters, etc.), the network environment 100 may include any number of components (in any combination) that are interconnected in any arrangement to facilitate the exchange of data between the computing devices.

The analytic server 102 may function as an interface for an administrator to set configuration settings or provide operational instructions to various components of the network environment 100. The analytic server 102 may be any computing device comprising a communications component capable of wired or wireless communication with other components of the network environment 100, and a microprocessor configured to transmit and receive certain types of data from the components of the network environment 100.

Non-limiting examples of the analytic server 102 may include a server (e.g., an application server, a catalog server, a communications server, a computing server, a database server, a file server, a game server, a mail server, a media server, a proxy server, a virtual server, a web server, etc.) a personal computer, a laptop computer, a desktop computer, a mobile computer, a tablet computer, a smart phone, a digital video recorder, a set-top box for a television, a video game console, a digital wallet (sometimes referred to as an “e-Wallet”), or any other type and form of computing device or combinations of devices. In some embodiments, the type of analytic server 102 may be categorized as a mobile device, a desktop device, a device intended to remain stationary, a device adapted to primarily access a network via a local area network (e.g., network 116), or another category of electronic devices such as a media consumption device. The analytic server 102 may include a user application (e.g., a web browser, an email application, an FTP application, etc.) to facilitate the sending and receiving of data over network 116.

For ease of explanation, FIG. 1A shows a single computer device functioning as the analytic server 102. However, it should be appreciated that some embodiments may comprise any number of computing devices functioning as the analytic server 102 and capable of performing the various tasks described herein

The analytic server 102 may receive information on wireless signals (sometimes referred to as, “signals”) detected by one or more observer devices 106 through a network 116 to generate one or more hyperclusters (sometimes referred to as, “proximal groupings of electronic devices”). The analytic server 102 may receive identification information about wireless signals (sometimes referred to as, “signals”) detected by one or more of the observer devices 106a, 106b. In response to receiving the identification information, the analytic server 102 may generate one or more hyperclusters using the identification information, and/or store the identification information and hyperclusters in the database 104 for further processing.

The analytic server 102 may explain the relationships in the physical world by measuring relationships between signals and devices. By approximating the world via temporal relationships between signals, the analytic server 102 may build a dataset to compute temporal persistence between devices. This dataset may be referred to as a signal graph (sometimes referred to as, “SignalGraph”). The signal graph is temporal graph model that may connect signals and observers into a network. The signal graph may comprise signals and observers (e.g., observer devices that observe the signals) at different time points.

The signal graph may provide information about relationships in the physical world. For example, the analytic server 102 may generate a set of hyperclusters (or signal clusters or proximal grouping of electronic devices) based on the spatial proximity and temporal persistence of the wireless signals. A hypercluster may be a set of signals that have been observed together within a number of observations. A given hypercluster may represent a set of devices that remain in physical proximity over time. In other words, a hypercluster is a static relationship between signals, as the proximity persists across time and observations.

The analytic server 102 may be directly or indirectly connected to observer devices 106a, 106b and database 104. Accordingly, the analytic server 102 may be capable of wired or wireless communication through a variety of communication channels with the observer devices 106a, 106b and the database 104 over a network 116. During the wired or wireless communication between the analytic server 102, the observer devices 106a, 106b, and the database 104, each of these devices may be capable to transmitting and receiving data from each other. In some embodiments, each of these devices may normalize and format the data in accordance to pre-stored instructions prior to transmitting the data to other devices. In some embodiments, each of these devices may store a local copy of the data in their memory prior to transmitting original copy of the data to other devices.

Examples of a network 116 may include, but are not limited to, private or public LAN, WLAN, MAN, WAN, and Internet. The network 116 may include both wired and wireless communications channels according to one or more standards and/or via one or more transport mediums. The communication over the network 116 between the components of the network environment 100 may be performed in accordance with various communication protocols such as Transmission Control Protocol and Internet Protocol (TCP/IP), User Datagram Protocol (UDP), and IEEE communication protocols. In one example, the network may include wireless communications according to Bluetooth specification sets, or another standard or proprietary wireless communication protocol. In another example, the network may also include communications over a cellular network, including, e.g., a GSM (Global System for Mobile Communications), CDMA (Code Division Multiple Access), and EDGE (Enhanced Data for Global Evolution) network.

Observer devices 106a, 106b may be any computing and/or telecommunications devices comprising a processor and capable of performing various tasks and processes described herein. Non-limiting examples of the observer devices may include a telephone 106a (e.g., smartphone), a user computer 106b (e.g., desktop, laptop, server, tablet), or any other telecommunications or computing device capable of performing the various tasks and processes described herein. For ease of explanation, FIG. 1A shows two devices functioning as the observer devices 106a, 106b. However, it should be appreciated that some embodiments may comprise any number of observer devices capable of performing the various tasks described herein.

In some embodiments, observer devices 106a, 106b may be computing devices that function as sensor devices, and are directly or indirectly associated with an analytic server 102 and/or a database 104. The sensor devices may be capable of observing signals in their zone of operation emitted by various devices such as IoT devices. The sensor device may further include a sensor processor configured to process the observed signals and extract identification information from the observed signals. Non-limiting examples of the sensor technologies for the sensor devices may include resonant LC sensors, capacitive sensors, and inductive sensors. Based upon the particular type of the sensor waves used and the particular protocols associated with the sensor waves, the sensor devices may observe signals and then generate sensor data, which may include information associated with the observed signals. The sensor processor may receive, interpret, and process sensor data, which the sensor may then provide to a processor of the analytic server 102 and/or the database 104.

Each observer device may include identification information. The identification information may include a name of the observer device, a type of the observer device, a model number of the observer device, a location information of the observer device, and an ID of the observer device where the ID may be pseudo-random identifier such as a hash value. In some cases, each observer device may have multiple IDs and the IDs may change at any time. All past and current identification information of each of the observer device may be stored in a database 104. For example, a given observer device may have an old ID and a new ID, and in such as case, both the old and new IDs may be stored in the database 104. The analytic server 102 may have access to the identification information of each observer device stored in a database 104. The analytic server 102 may generate a query and/or a request and transmit the query and/or the request at any time to the database 104 to receive identification information of any observer device. In some cases, the analytic server 102 on receiving signal data from the observer device may query the database 104 to receive additional identification information regarding the observer device from which it received the signal data.

The analytic server 102 may set configuration settings or provide operational instructions to observer devices 106a, 106b to make observations of signals transmitted by various devices such as Internet of Things (IoT) devices and then provide analytics and data about signal observation application activity back to the analytic server 102. In some embodiments, the analytic server 102 may generate and transmit the operational instructions to the observer devices 106a, 106b at any point of time in order to enable the observer devices 106a, 106b to make the observations of the signals transmitted by various devices such as IoT devices, and then provide analytics and data about signal observation application activity back to the analytic server 102. In some embodiments, the analytic server 102 may generate and transmit the operational instructions to the observer devices 106a, 106b at any point of time in order to disable the observer devices 106 from making any observations of the signals transmitted by various devices such as IoT devices, and then notify the successful disablement of the observer devices 106a, 106b back to the analytic server 102. In some embodiments, the analytic server 102 may also transmit a weblink of configuration settings to the observer devices 106a, 106b, and the observer devices 106a, 106b may use the weblink for installation of the configuration settings in their hardware and/or software. The configuration settings may enable or disable the observer devices 106a, 106b to make the observations of the signals transmitted by various devices such as IoT devices, and then provide analytics and data about signal observation application activity back to the analytic server 102. In some cases, the configuration settings may enable the observer devices 106a, 106b to make the observations of the signals transmitted by various devices such as IoT devices for a limited period of time (such as 2 hours a day) in the day, and the same configuration settings may also disable the observer devices 106a, 106b from making any observations of the signals during the rest of the day. In some cases, the configuration settings may disable the observer devices 106a, 106b from making any observations of the signals when their battery charge is below a predetermined threshold. For this purpose, the configuration settings may allow the analytic server 102 to constantly monitor battery charge of the observer devices 106a, 106b and when the battery charge is below a predetermined threshold, and then the analytic server 102 may disable the observer devices 106a, 106b from making any observations of the signals. In some cases, the configuration settings may disable some applications of the observer devices 106a, 106b when their battery charge is below a predetermined threshold to allow the observer devices 106a, 106b from making observations of the signals. In some embodiments, the configuration settings may instruct the observer devices 106a, 106b to send to the analytic server 102 signals associated with specific types of devices or entities such as signals associated with businesses or other enterprises. The configuration settings may instruct the observer devices 106a, 106b not to send signals associated with individuals' personal devices (e.g., fitness trackers) to the analytic server 102.

The analytic server 102 may receive data including wireless signals detected by observer devices 106a, 106b. In some embodiments, the observer devices 106a, 106b may transmit the data including observed signals to the analytic server 102 as soon as the analytic server 102 detects any signals. In some embodiments, the observer devices 106a, 106b may transmit the observed signals to the analytic server 102 after a predetermined period of time. For example, the observer devices 106a, 106b may be programmed to periodically (e.g., daily) transmit data including all observed signals to the analytic server 102. In some embodiments, the analytic server 102 may fetch data including the observed signals data from the observer devices 106a, 106b periodically (e.g., daily). In some embodiments, the analytic server 102 may fetch data including the observed signals data from the observer devices 106a, 106b based on a triggering condition (e.g., time-based periodic updates, real-time updates). The data may include, but may not be limited to, all observed wireless signals, a time point at which each wireless signals was observed, approximate latitude coordinates of where event of observation is recorded, approximate longitude coordinates of where event of observation is recorded, among other data and identification information.

The analytic server 102 may store into the database 104 all the observation data, such as observed wireless signals, a signal strength value (e.g., RSSI), a time point at which each wireless signals was observed, and, in some implementations, approximate latitude coordinates of where event of observation is recorded, and approximate longitude coordinates of where event of observation is recorded in a database 104 for further processing. In some embodiments, the analytic server 102 may store all the data in the database 104 in a format in which all the data was received by the analytic server 102. In some embodiments, the analytic server 102 may first normalize and format all the data, and then store the normalized and formatted version of the data in the database 104. The analytic server 102 may use any suitable normalization and formatting technique to normalize and format all the data depending on content, received format, structure, and size of the data. Upon normalization and formatting of the data, the analytic server 102 may execute algorithms such as clustering algorithms to generate one or more hyperclusters of the signal datasets. Each hypercluster may represent a set of signals that have been observed together by the observer devices 106a, 106b within a number of observations made by the observer devices 106a, 106b. In some cases, for every two observations in the hypercluster, there may exist at least two overlapping observations that contain said two observations.

As illustrated in FIG. 1A, a first observer device 106a (e.g., a smartphone, a tablet, or other device, etc.) may detect, at timepoint_1, wifi_signal_1 generated by a first Wi-Fi router 108 and wifi_signal_2 generated by a second Wi-Fi router 110. A second observer device 106b (e.g., a smartphone, a tablet, or other device, etc.) may detect, at timepoint_2, wifi_signal_1 generated by the first Wi-Fi router 108, bluetooth_signal_4 generated by Bluetooth transmitter 114, BLE_signal_3 generated by a Bluetooth low energy (BLE) transmitter 112. Furthermore, the first observer device 106a may detect, at timepoint_3, the BLE_signal_3 generated by the BLE transmitter 112. Each of the aforementioned signals may include a tuple of (name, MAC address, type). Two signals may be equivalent of all three elements are equivalent.

Each observer device 106a, 106b may transmit through the network 116 information of the detected signals to the analytic server 102 for storage in the database 104 and for further analysis. Based on the temporal persistence and spatial proximity of the signals observed by the observer devices 106a, 106b and received by the analytic server 102, the analytic server 102 may define one or more hyperclusters (or proximal groupings of electronic devices) associated with the location where the signals are received from.

A data model employed by the analytic server 102 to identify the hyperclusters (e.g., proximal groupings of electronic devices) may include a set of signals S observed by a population of observer mobile devices U. In the illustrative network environment 100a, S={wifi_signal_1, wifi_signal_2, BLE_signal_3, bluetooth_signal_4} and U={106a, 106b}. As described above, each of the signals in the set of signals S may include a tuple of (name, MAC address, type). The analytic server 102 may identify each observer device 106 with a respective mobile advertising identifier or any other identifier assigned to or associated with the app or observer device 106 (e.g., observation identifier), abbreviated as adid or obsvID. The analytic server 102 may associate each adid of the observer devices 106a, 106b with a matrix of signals and time points. More specifically, the analytic server 102 may construct a sparse Boolean matrix to denote which signals an observer adid observed in a given time window. In other words, the Boolean matrix for the observer device 106a, 106b may indicate a presence of (indicated by entry 1) or absence of (indicated by entry 0) one or more signals, as detected by the observer device 106a, 106b for a particular time period. The analytic server 102 may, however, discard signals at stale time points as reported by the observer devices 106a, 106b even though the stale time points may not indicate a nefarious behavior. For example, if an observer device 106a, 106b has a single observation that stretches credulity (threshold set at more than five days lag), the analytic server 102 may simply remove the observation. In some embodiments, the observer devices 106a, 106b may also transmit the respective latitude longitude coordinates of the observer devices 106a, 106b. In some instances, the observer devices 106a, 106b may observe the wireless location signals from coinciding locations. For example, the latitude longitude coordinates of the observer devices 106a, 106b may be the same.

Based on the analysis of the matrices associated with the observer devices 106, the analytic server 102 may generate one or more hyperclusters based on the temporal persistence and spatial proximity of the received signals. For example, FIG. 1B illustrates a network environment 100b which may be useful for practicing embodiments described herein, according to an embodiment. That is, a network environment 100b may include a hypercluster 118 that is generated by the analytic server 102 based on the wireless signals detected by the observer devices 106. In this illustration, the hypercluster 118 may contain three wireless signals: wifi_signal_1, wifi_signal_2, bluetooth_signal_4. The analytic server 102 may determine the spatial proximity of wifi_signal_1, wifi_signal_2, bluetooth_signal_4 based on the fact that the these signals were detected simultaneously or near-simultaneously by the observer devices 106a, 106b. The analytic server 102 may determine the temporal persistence of wifi_signal_1, wifi_signal_2, bluetooth_signal_4 based on the fact that the two observer devices 106a, 106b observed these signals at two time points: the first observer device 106a observed these signals at timepoint_1 and the second observer device 106b observed these signals at timepoint_2. However, the analytic server 102 may determine that BLE_signal_3, even though having spatial proximity with wifi_signal_1, wifi_signal_2, bluetooth_signal_4 may not have the requisite temporal persistence. For example, the first observer 106a did not detect BLE_signal_3 at timepoint_1.

The observer devices 106a, 106b may be directly or indirectly connected to the analytic server 102 and a database 104. Accordingly, the observer devices 106a, 106b may be capable of wired or wireless communication through a variety of communication channels with the analytic server 102 and the database 104 over a network 116. During the wired or wireless communication between the observer devices 106a, 106b, the analytic server 102, and the database 104, each of these devices may be capable to transmitting and receiving data from each other. In some embodiments, the observer devices 106 may normalize and format the data in accordance to pre-stored instructions prior to transmitting the data to the analytic server 102 and/or the database 104. In some embodiments, the observer devices 106a, 106b may store a local copy of the data in their memory prior to transmitting original copy of the data to the analytic server 102 and/or the database 104.

The observer device 106a, 106b may be configured to observe an event. The event may contain all signals that the observer device 106a, 106b scanned around its zone of operation at a given time point. Accordingly, the event may include observed signal data, and in some cases, the event may also include approximate or correct values of latitude coordinates of where the event is recorded by the observer device 106a, 106b at a given time point. In some cases, the event may further include approximate or correct values of longitude coordinates of where the event is recorded by the observer device 106a, 106b at a given time point.

The event is caused when observer device 106a, 106b observes signals from various devices such as IoT devices. The signals may be an electromagnetic signal emitted by the IoT devices. It is to be noted that the signal may be any type of signal emitted by the IoT devices without moving out the scope of the disclosed embodiments. The signals observed by the observer device 106a, 106b may represent discrete values about the signals. In some embodiments, the discrete values of the signals may be characterized by a type of signal. The type of signal may include, but may not be limited to, a Bluetooth® signal, wireless fidelity (Wi-Fi) signal, or Bluetooth Low Energy (BLE) signals. In some embodiments, the discrete values of the signals may further be characterized by a name of signal. The name of the signal may be a SSID (service set identifier) that identifies an IoT device. The SSID may be a unique ID that consists of 32 characters and is used for naming wireless networks. In some embodiments, the discrete values of the signals may further be characterized by an address of the IoT device through which the device communicates the signal. Each IoT device may emit multiple signals.

Network components may effectuate wired and/or wireless signal communications to and from various devices. The network components may include transmitters, a first Wi-Fi router 108, a second Wi-Fi router 110, and a Bluetooth low energy (BLE) transmitter 112. These network components may be an embedded component of an electronic device; and, in some cases, the network component may be attached to the electronic device through any wired or wireless communications medium. The network components such as the first Wi-Fi router 108, the second Wi-Fi router 110, and the Bluetooth low energy (BLE) transmitter 112 may include electromechanical components (e.g., processor, antenna) that allow the network components to communicate various types of signal data with one or more electronic devices. In some implementations, these signals may represent a distinct channel for hosting communications. The data may be communicated using signals, based on predetermined wired or wireless protocols and associated hardware and software technology. The network components may operate based on any number of communication protocols, such as Bluetooth®, Wireless Fidelity (Wi-Fi), and others.

Databases 104 may be directly or indirectly connected to observer devices 106a, 106b and an analytic server 102. Accordingly, the database 104 may be capable of wired or wireless communication through a variety of communication channels with the observer devices 106a, 106b and the analytic server 102 over a network 116. During the wired or wireless communication between the analytic server 102, the observer devices 106a, 106b, and the database 104, the database 104 is capable of receiving data from the analytic server 102 and the observer devices 106. The data may include, but may not be limited to, all observed wireless signals, a time point at which each wireless signals was observed by the observer devices 106a, 106b, approximate latitude coordinates of where event of observation is recorded by the observer devices 106a, 106b, approximate longitude coordinates of where event of observation is recorded by the observer devices 106a, 106b, among other data and identification information. For ease of explanation, FIG. 1A shows a single database 104. However, it should be appreciated that some embodiments may comprise any number of databases capable of performing the various tasks described herein.

The database 104 may have a logical construct of data files that are stored in non-transitory machine-readable storage media, such as a hard disk or memory, controlled by software modules of a database program (for example, SQL), and a related database management system (DBMS) that executes the code modules (for example, SQL scripts) for various data queries and other management functions generated by the analytic server 102 and the observer devices 106a, 106b. In some embodiments, a memory of the databases 104 may be a non-volatile storage device. The memory may be implemented with a magnetic disk drive, an optical disk drive, a solid-state device, or an attachment to a network storage. The memory may include one or more memory devices to facilitate storage and manipulation of program code, set of instructions, tasks, data, PDKs, and the like. Non-limiting examples of memory implementations may include, but are not limited to, a random access memory (RAM), a read only memory (ROM), a hard disk drive (HDD), a secure digital (SD) card, a magneto-resistive read/write memory, an optical read/write memory, a cache memory, or a magnetic read/write memory. In some embodiments, a memory of the databases 104 may be a temporary memory, meaning that a primary purpose of the memory is not long-term storage. Examples of the volatile memories may include dynamic random access memories (DRAM), static random access memories (SRAM), and other forms of volatile memories known in the art. In some embodiments, the memory may be configured to store larger amounts of information than volatile memory. The memory may further be configured for long-term storage of information. In some examples, the memory may include non-volatile storage elements. Examples of such non-volatile storage elements include magnetic hard discs, optical discs, floppy discs, flash memories, or forms of electrically programmable memories (EPROM) or electrically erasable and programmable (EEPROM) memories.

In operation the analytic server 102 may utilize the hypercluster 118 (or proximal grouping of wireless devices 108, 110, 114) to generate a semantic to one or more wireless signals. For example, if the hypercluster 118 is observed by multiple devices throughout the day and not observed by any device during the night, the analytic server 102 may determine that the hypercluster 108 may be within an office and the devices 108, 110, 114 may be installed in an office. If the hypercluster 118 is persistently observed by a few devices, the analytic server 102 may determine that the hypercluster 108 may be within a home and the devices 108, 110, 114 may be installed in a home. The analytic server 102 may also distinguish between enterprise and business signals (e.g., router associated with a company or a chain location) and personal signals (e.g., a BLE signal emitted by a person's fitness tracker). In particular, the analytic server 102 may include a trained classifier to classify whether an observed signal is associated with a particular location (e.g., cafeteria, conference room, gym) or a person. Such classification may allow the analytic server 102 to perform operations in a manner that respects individuals' expectation of privacy and complies with privacy rules in different jurisdictions.

FIG. 1C illustrates a network environment 100c for locating and/or tracing interactions between electronic devices, according to an embodiment. The analytic server 102 may receive an indication from another server (not shown) that a mobile app has been installed on the observer electronic device 120. In response, the analytic server 102 may communicate with the observer electronic device 120 or one or more apps installed therein to trace the interactions of the observer electronic device 120 with one or more signal environments. The observer electronic device 120 may interact with the signal environment by detecting wireless signals around the environment. For example, the observer electronic devices 120 may detect wireless signal from a target device 121 and other wireless signals from signal clusters (also sometimes referred to as, “hyperclusters” and/or “proximal groupings of electronic and/or wireless devices”) 124a, 124b, 124c, 124d. The analytic server 102 may store the interactions information into the database 104. Based on these interactions, the analytic server 102 may determine the location of the observer electronic device 120. Assuming the observer electronic device 120 is in proximity to the target device 121, the analytic server may determine the location of the target device 121. In particular, the analytic server 102 may determine the hyperclusters the observer electronic device 120 detected. As the target device 121 moves, the observer electronic device 120 that detects the wireless signals emitted from the target device 121 may detect different hyperclusters within the same geolocation or from different geolocations. For example, signal clusters 124a, 124b may be co-located at the same or nearly same geolocation 130a. Furthermore, signal clusters 124c, 124d may be co-located at the same or nearly same geolocation 130b. As shown herein, the analytic server 102 may observe four changes in signal context: hyperclusters 124a, 124b, 124c, 124d. However, these four hyperclusters may be associated with two geolocations 130a, 130b. In other words, the same geolocation may include multiple hyperclusters with each hypercluster corresponding to a micro-location within the same geolocation. Thus, the hyperclusters may be able to provide more accurate and fine-grained location information. For example, hyperclusters may be sufficient to identify a specific location in a tall building while geolocation of a tall building may not be sufficient.

When the analytic server 102 receives a request indicating that the target device 121 is lost, the analytic server 102 may locate the observer device 120 that detects wireless signals emitted from the target device 121 based on the signal environment the observer electronic device 120 is in. The analytic server 102 may receive the request to find the a target device 121, and receive observation data from the observer electronic device 120 or a plurality of other electronic devices (not shown) that observe/sense wireless signals emitted from the target device 121. Based on the observations comprising the wireless signal from the target device 121 and other wireless signals within the environment, such as wireless signals from a BLE transmitter 122a and a Wi-Fi router 122b, the analytic server 102 may determine, for example, that the observer electronic device 120 and the target device 121 are proximately located and in the signal context of hypercluster 124c, and in some cases, within a geolocation 130b.

Furthermore, the analytic server 102 may perform a resolution of the wireless signals from the BLE transmitter 122a and/or the Wi-Fi router 122b within hypercluster 124c to assign semantic meanings or atoms to the wireless signals. The semantic meanings or atoms may be knowledge on a given signal in terms of business, manufacture, function, location, and the like. Such wireless signals may provide more information on the location or environment the target device 121 is located in. For example, the analytic server 102 may resolve the wireless signals from the BLE transmitter 122a and the Wi-Fi router 122b within hypercluster 124c. The resolution results of the wireless signals may include the location, business, and any other knowledge associated with the wireless signals. For example, the resolution results may indicate that these signals are from a coffee store (e.g., Starbucks®). Such information may further narrow down the mobile device's location. For example, based on the geolocation, the analytic server may be able to determine a specific building; based on the semantic meanings (e.g., business), the analytic server may be able to determine a specific store in the building.

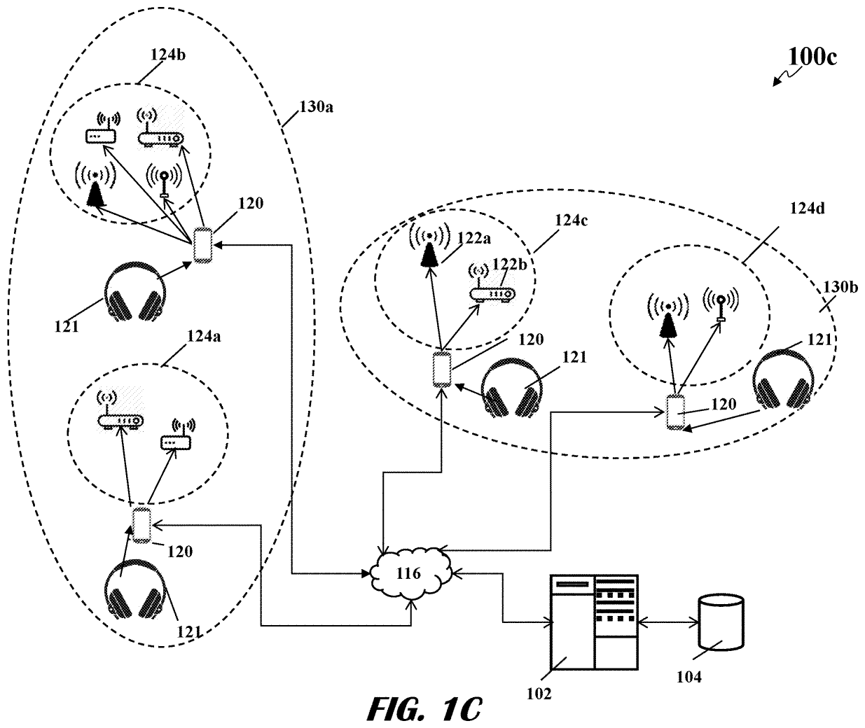

FIG. 2 shows a flow diagram 200 of a method for tracing and locating electronic devices, according to an illustrative embodiment. Other embodiments may comprise additional or alternative operations, or may omit some operations altogether. Although multiple computing systems and databases can implement one or more operations of the method, this description details, for brevity, an analytic server implementing the various operations of the method.

At operation 202, the analytic server may monitor wireless signals detected by a plurality of electronic devices to generate hyperclusters (also referred to as proximal groupings of wireless devices). In operation, the analytic server may trigger a signal scanning function on the electronic devices. An electronic device may be a mobile device (or handheld computer) that is portable enough to hold and operate in the hand. Typically, any handheld computer device will have a liquid-crystal display (LCD) flat screen interface, providing a touchscreen interface with digital buttons and keyboard or physical buttons along with a physical keyboard. Many such devices can connect to the Internet and interconnect with other devices such as car entertainment systems or headsets via Wi-Fi, Bluetooth, cellular networks or near field communication (NFC). Mobile devices may run mobile operating systems that allow third-party apps specialized for said capabilities to be installed and run. In the embodiments disclosed herein, the users of the electronic devices may install a software application from a vendor and the analytic server may receive the notifications from one or more servers of the vendor. The installation of the software application may trigger a signal scanning function on the electronic devices. The signal scanning function may enable the electronic devices to detect different wireless signals around the electronic device. The wireless signals may comprise Wi-Fi, Bluetooth, and Bluetooth Light (BLE). The installation of the software application may also trigger the electronic devices to transmit the detected wireless signals to the analytic server. For instance, the electronic devices may transfer a tuple of (name, MAC address, type) for the detected signals.

The analytic server may monitor wireless signals detected by the plurality of electronic devices. The analytic server may collect the wireless signals detected/observed by the electronic devices at different time points. In some embodiments, the plurality of electronic devices may also report the geolocation data, such as latitude and longitude coordinates of where the observation is recorded. The analytic server may store the observations of the wireless signals and the geolocation data (if available) into a database.

The analytic server may collect the wireless signals detected/observed by the electronic devices at different time points periodically. For example, the analytic server may query the detected wireless signals from the electronic devices every minute. The analytic server may monitor the electronic devices for a predetermined time window (e.g., a sliding window). For example, the analytic server may monitor the electronic devices for seven days or three months.

The analytic server may monitor a given population of electronic mobile devices (users). Each electronic mobile device reports the detected signals to the analytic server. Let S denote a set of signals observed by the given population of mobile devices. As described above, a signal s may be a tuple (name, MAC address, type). The analytic server may consider two signals to be equivalent if all three elements are equivalent. Each mobile user may be identified with a mobile advertising identifier, sometimes abbreviated as adid. Different adids may represent different mobile phones and users. Each adid may be associated with a matrix of signals and time points. Each row is a signal in S, while time points T are of minute precision, and may be closed by a given time window for the analysis. The analytic server may construct a sparse Boolean matrix, U→Bool^{S×T} to store which signals the mobile adid u observed in the given time window. If a mobile device observed a signal s at time t, the analytic server may set the corresponding element in the matrix to 1; otherwise, set the element to 0.

In some embodiments, electronic devices report time points that may become stale over a few days. Whether or not this is indicative of nefarious behavior, doing time-dependent signal analysis on an observer's (e.g., the mobile phone's) signal observations may be difficult for the analytic server if their times are overly stale. If an observer had a signal observation that stretches credulity (the threshold set at more than five days lag), the analytic server may remove that observation (e.g., the detected wireless signals). In some embodiments, the analytic server may remove from consideration an observer (e.g., mobile device) with two or more incredible time points.

The analytic server may analyze the wireless signals to generate hyperclusters. More specifically, the analytic server may analyze the wireless signals collected at different time points from different electronic devices to build the signal graph and generate a set of hyperclusters (or signal clusters) based on the spatial proximity and temporal persistence of the wireless signals. The analytic server may save the signal graph, the hyperclusters, and corresponding geolocation data into the database.

As discussed above, the analytic server may build the signal graph that connects signals and observations into a network. The signal graph may comprise signals and observers (e.g., observer devices that observe the signals) at different time points. The analytic server may generate a set of hyperclusters (or signal clusters) based on the spatial proximity and temporal persistence of the wireless signals. A hypercluster may be a set of signals that have been observed together within a number of observations. A given hypercluster may represent a set of devices that remain in physical proximity over time. In other words, a hypercluster is a static relationship between signals, as the proximity persists across time and observations. The hyperclusters may provide useful information (e.g., location) on the physical world in terms of signals. The analytic server may utilize such information to determine the movement or location changes of a mobile device.

At operation 204, the analytic server may receive a request to locate an electronic device. The request may comprise the identifier of the target device. The identifier may be BLE standard unique identifier. Alternatively, the identifier may be MAC (media access control) address. In operation, a user may open a website in an Internet browser or a local application on an electronic client device configured to receive a request from the user. The analytic server may display a graphical user interface (GUI) for the user to input the request. For example, the user interface may include a text-based interface where the user can manually type requests and provide identifiers of target devices using a keyboard. In another example, the user interface may include an audio-based interface where the user can issue requests by verbally requesting a service.

In some embodiments, instead of looking for an electronic device, the analytic server may actively monitor the electronic device and report the locations of the electronic device by triggering an electronic message. In operation, the analytic server may provide the option to turn on or turn off an alert-triggering mechanism in the GUI. When the user turns on the alert-triggering mechanism, the analytic server may either periodically report the locations of the electronic device or trigger alert electronic messages when the device is acting out of pattern, or has moved out of a safe zone. For example, the analytic server may determine the changes of the hyperclusters of the electronic device, and transmit an alert electronic message when the changes of the hyperclusters satisfy a threshold. The alert electronic message may be proactive messages including instant messages, SMS, emails, text message, phone calls, and the like. More specifically, the analytic server may monitor the signal environment (e.g., hyperclusters) of the electronic device and determine the locations of the electronic device based on the observations from a plurality of observer devices that detect the wireless signals emitted by the electronic device. The analytic server may periodically report the locations of the electronic device. Alternatively, the analytic server may determine if the electronic device is acting out of pattern by determining how likely the device has changed dramatically, and only trigger an alert when the electronic device is acting out of pattern. However, the device may act out of pattern legitimately. For example, a luggage with BLE communication capability may be in home most of time, and start acting out of pattern when the user is travelling. The analytic server may need to be able to determine that such out of pattern actions are legitimate based on the user's reaction to the alert electronic message. In some embodiments, an administrator may configure the system and user devices to only operate within a predefined geofence based on configuration settings (sometimes referred to as, “configuration data”) of a polygon. That is, the configuration settings instructs the device to begin transmitting the observation data only when it is within a virtual parameter as defined by the configuration settings of the polygon.

In some embodiments, an administrator may configure the system and user devices (e.g., observation devices) to only operate within a predefined geofence based on configuration settings of a polygon. That is, the system instructs the device to begin transmitting the observation data only when the device is within a virtual parameter of the polygon.

At operation 206, the analytic server may receive observations comprising the wireless signals emitted by a target electronic device from the plurality of electronic devices. The plurality of electronic devices may act as a network of sensors or observers for the target device. The analytic server may monitor wireless signals detected by the plurality of electronic devices by continuously receiving the wireless signals. Once the analytic server receives the request to trace interactions involving a target device, the analytic server may retrieve observation data including the wireless signals detected by different devices and determine which devices sensed the wireless signals emitted by the target device. The observer devices may also observe other wireless signals around the environment. The analytic server may further analyze the observations from such observer devices to determine, for example, the location information.

An observation may comprise detected Wi-Fi, Bluetooth, and/or BLE signal identifiers, including SSID (e.g., signal name), MAC address and/or universally unique identifier (UUID), tech (Wi-Fi, Bluetooth, BLE) and RSSI (relative measure of signal strength). Each observation may also include the following fields: token, a unique key provided by the analytic server to the app developer; ID, such as Google adid or iOS idfa (advertising identifier), the pseudorandom, resettable advertising identifier attributed to a smartphone by its operating system; the freshest available latitude/longitude reading from the smartphone; timestamp, data and time of the observation; metadata including SDK (software development kit) version, app name, device model and manufacture, and a tag assigned to the app by the developer. Assuming the observer electronic devices are within the same location of the target device, the analytic server may leverage the observer electronic devices to determine the extent of interactions between the devices of the target device (e.g., distance, duration), and thus infer the amount of contact and exposure between humans who are associated with those observer devices. The analytic server may query and refresh the observation input from the observer devices as frequently as possible or may receive a stream of observation data from the observer devices via one or more networks.

At operation 208, the analytic server may determine the observer device that were in contact with, and in some cases a geolocation or a hypercluster, of the target device based on the observations from the plurality of observer devices. The observation of each observer device may comprise the wireless signals of the target device and other wireless signals from the surrounding environment. As discussed above, the analytic server may build hyperclusters based on the monitoring of the plurality of electronic devices. Based on the observations from the plurality of observer devices, the analytic server may determine the signal context, such as hypercluster, of the target device by determining the hypercluster of the observer devices that detect wireless signals from the target device. For example, the analytic server may query the list of detected wireless signals from the observer devices and determine the corresponding hypercluster. In some other embodiments, the analytic server may be able to receive the geolocation directly from the observer devices when GPS (global positioning system) data is available.

The hypercluster may provide information on the signal environment of the target device and relevant observer devices, and further provide information on the physical environment, such as, for example, the intensity (e.g., business) that observer devices are seen at a location (in a hypercluster), or the geolocation of traced interactions. Because a hypercluster may be a set of signals that have been observed together within a number of observations, a given hypercluster may represent a set of devices that remain in physical proximity over time. In other words, a hypercluster may correspond to a physical location of note, such as a particularly busy public space or an employee's office. The analytic server may determine the physical location based on the hypercluster. For example, the analytic server may retrieve a database storing the hyperclusters and the corresponding locations.

In some embodiments, the analytic server may monitor the plurality of electronic devices, build the signal graph, and analyze the wireless signals from the plurality of electronic devices in the signal graph to determine the hyperclusters in an on-demand mode, which may provide results that are more precise. In some other embodiments, the analytic server may determine the hyperclusters in a streaming mode, for example, the analytic server may build the hyperclusters per day.

At operation 210, the analytic server may optionally use additional types of data or calculations to, for example, determine a geolocation a traced interaction or determine the user was in a particularly busy location or hypercluster based on observation data indicating semantic meanings of the wireless signals of the hypercluster. The analytic server may perform resolution of the wireless signals to assign semantic meanings or atoms to the wireless signals based on, for example, predetermined data values corresponding to observer identifiers. The semantic meanings or atoms may be knowledge on a given signal in terms of business, manufacture, function, location, and the like. Such wireless signals may provide more information on the location or environment the target device interacted with another observer device. The additional data or calculations may, for example, result in the analytic server identifying weights or business rules associated with a given location or hypercluster that are then referenced when calculating a risk score for the observer device that came into contact with the target device.

For example, the analytic server may resolve the wireless signals of the hypercluster. The resolution results may indicate that these signals are from a coffee store (e.g., Starbucks). Such information may further narrow down the mobile device's location. Based on the geolocation, the analytic server may be able to determine a specific building; based on the semantic meanings (e.g., business), the analytic server may be able to determine a specific store in the building.

The analytic server may return the following data regarding the target device: freshness, date and time of last observation; latitude/longitude, freshest reported latitude/longitude coordinates; cluster, identifier information about wireless signals observed in proximity to the targeted device, atoms, attributes for signals within the cluster to describe a venue or location (e.g., Starbucks).

At operation 212, the analytic server may generate a graphical user interface on the electronic client device comprising risk scores for the observer devices that were in contact with the target device, and in some implementations, location information of the target device. The output may include, for example, observer identifiers, employee names, a risk score, or a risk tier for each of the observer devices.

In some embodiments, the analytic server may work with a third-party server, such as a third-party company, to trace the locations of devices associated with the third-party company. The analytic server may receive a request from the third-party company to find one of its devices, perform the analysis to determine the device's location, and send the output of the device's location to the third-party company. The third-party company may then generate a graphical user interface via an app from the third-party company on a user's device. In other words, the analytic server may transmit the location information directly to a user's device and display the GUI comprising the location information on the user's device. Alternatively, the analytic server may work with a third-party company, which is in the middle between the analytic server and the user, and transmit the location information to the third-party company.

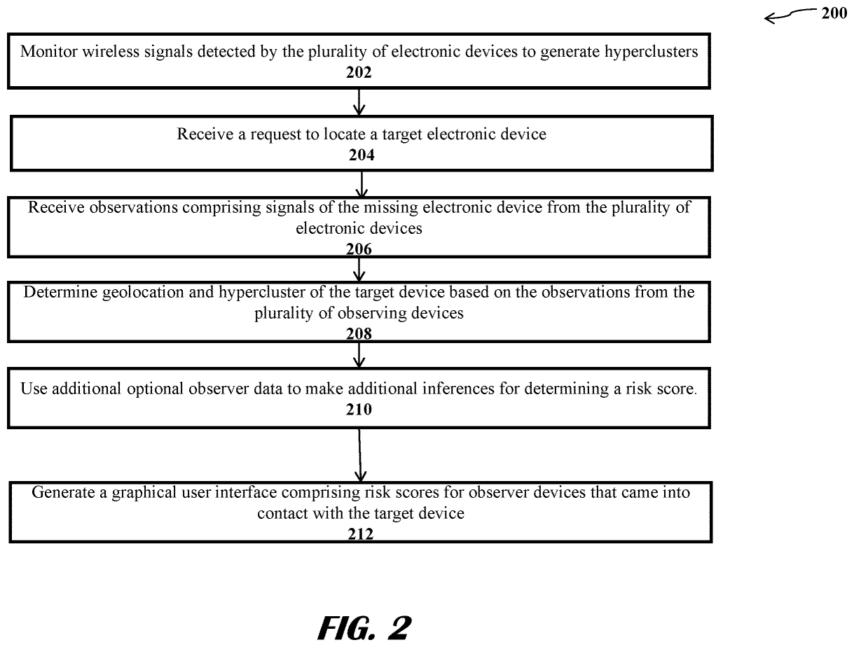

FIG. 3 illustrates an example of system architecture for working with a third party to locate electronic devices, according to an embodiment. In some embodiments, the analytic server 302 may work with an enterprise administration server 304, to trace interactions among electronic devices operated by the employees associated with the enterprise network or ecosystem. For example, employees may have their mobile devices 306 installed with an application (“app”) containing an SDK and developed for the enterprise server 304. This app may report signal observation data to the analytic server 302. The signal observation data may comprise signals and observers (e.g., observer devices that observe the signals) at different time points. Based on such signal observation data, the analytic server may build the signal graph and generate hyperclusters. In addition, the app on the mobile devices 306 may report additional data to the enterprise server 304, such as the observer identifier, a human employee identifier, and in some cases, a health status indicator to indicate the employee has been infected. An administrator user may issue a tracing request to the third-party server 304 via a client computing device (not shown) or submit the request directly into the third-party sever 304. The request may comprise the unique observer identifier of a target device associated with an employee who has test positive for infection. After receiving the request from the administrator, the enterprise server 304 may issue a tracing request to the analytic server 302 comprising the observer identifier of the target observer device. The analytic server 302 may identify which other observer devices in the network ecosystem were in contact with the target device and calculate a risk score for those devices. The analytic server may return the risk score or contact observation information to the enterprise server 304 as a response. The enterprise server 304 may receive the location information from the analytic server 302, transmit the location information, and generate a GUI for presentation to the administrator user's computing device to display the resulting tracing information. In some embodiments, the enterprise server 304 may enrich location information with map data from another server (not shown), based on latitude and longitude data received from the analytic server 302.

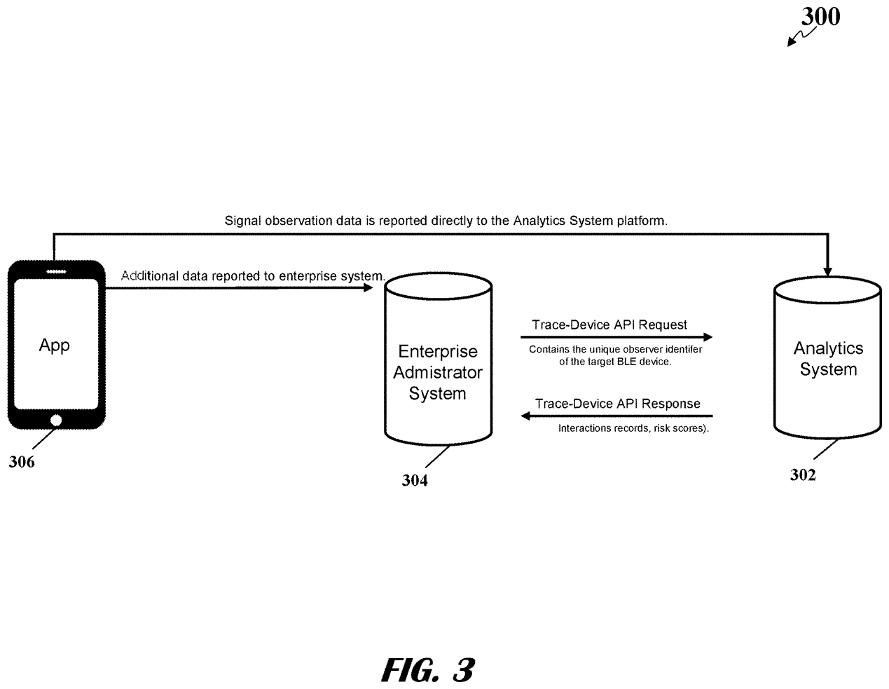

FIG. 4 illustrates a first example of data flows for locating electronic devices, according to an embodiment. The analytic server 402 may receive and collect observation data from mobile devices installed with a third-party app. The mobile devices 404 with the app may be devices operated by customers of the third-party server. The app may comprise observer SDK that collect observations of nearby, detectable Wi-Fi, Bluetooth, and BLE signal information from different devices, such as Wi-Fi hotspots, wearables, and smart-home electronics. The SDK-enabled app on mobile device 404 may report signal observation to the analytic server 402 through Hypertext Transfer Protocol Secure (HTTPS). The analytic server 402 comprising the signal graph platform may transform, encrypt, and index the input signal information (e.g., observations) into a knowledge-base. The analytic server 402 may comprise a query layer that receives a request from the third-party server 406 to find one of its devices. The analytic server may perform the analysis utilizing the combined network input and knowledge-base from the third-party server and other apps of signal clusters to determine the device's location, and send the response of the device's location to the third-party server.

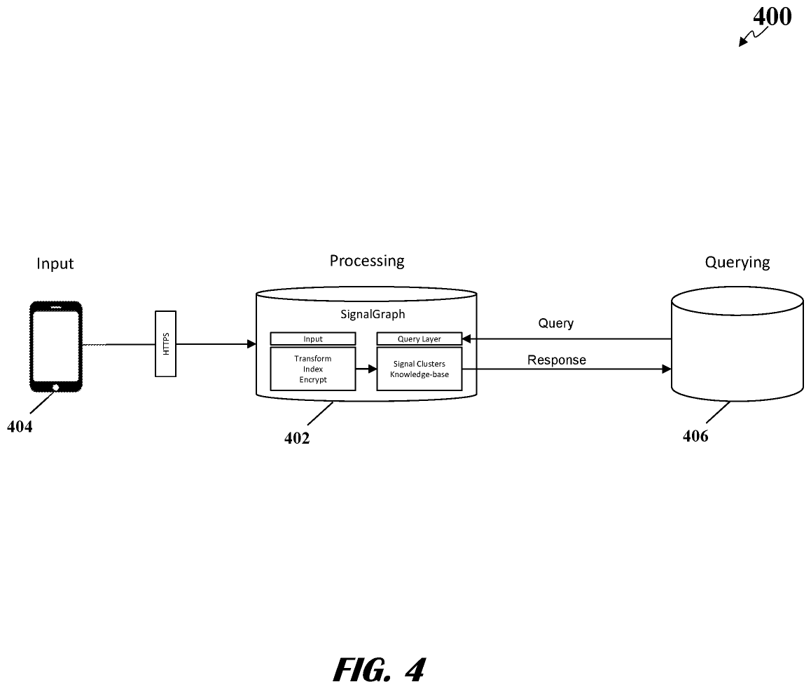

FIG. 5 illustrates a second example of data flows for locating (e.g., tracing) electronic devices, according to an embodiment. A mobile device 502 with a third-party server developed app may collect and report signal information from nearby devices (MACs, SSIDs, UUIDs), along with latitude/longitude and timestamp to a third-party server 504 through HTTPS. The third-party server 504 may validate the input and push all observations into a Kinesis queue. The Kinesis queue may hold observations for a period of time in a first come first out manner. The third-party server 504 may pass the observations (e.g., adid of smartphone, MACs, SSIDs, and UUIDs of observed signals, timestamp, and latitude/longitude, etc.) to the analytic server 506 through an observer application programming interface (API). The analytic server 506 may transform, index, and encrypt the input signal information (e.g., observations) into signal clusters. An OEM (Original Equipment Manufacturer) app end user may input a locate request (e.g., find-device request) on a user device 508. A proxy server 510 may perform request management for account authentication, and forward the request to the analytic server 506. The analytic server 506 may comprise a query layer that receives the request. The analytic server 506 may perform the analysis utilizing the combined network input and knowledge-base from the third-party server and other apps of signal clusters to determine the device's location. The analytic server 506 may return a response comprising freshest signal cluster, location, and timestamp. The proxy server 510 may enrich the response on mapping layer to include enriched location, POI and address information, and return the response to the end user device 508.

### II. Generating Risk Scores Based on Wireless Signals

Embodiments herein describe systems and methods for generating a risk score for a client device based on the wireless signals that are emitted from one or more wireless devices (e.g., other client devices, IoT devices, etc.). The risk score is an indication as to whether the client device was within a predetermined distance from the one or more wireless devices for a predetermined amount of time and/or a predetermined number of times.

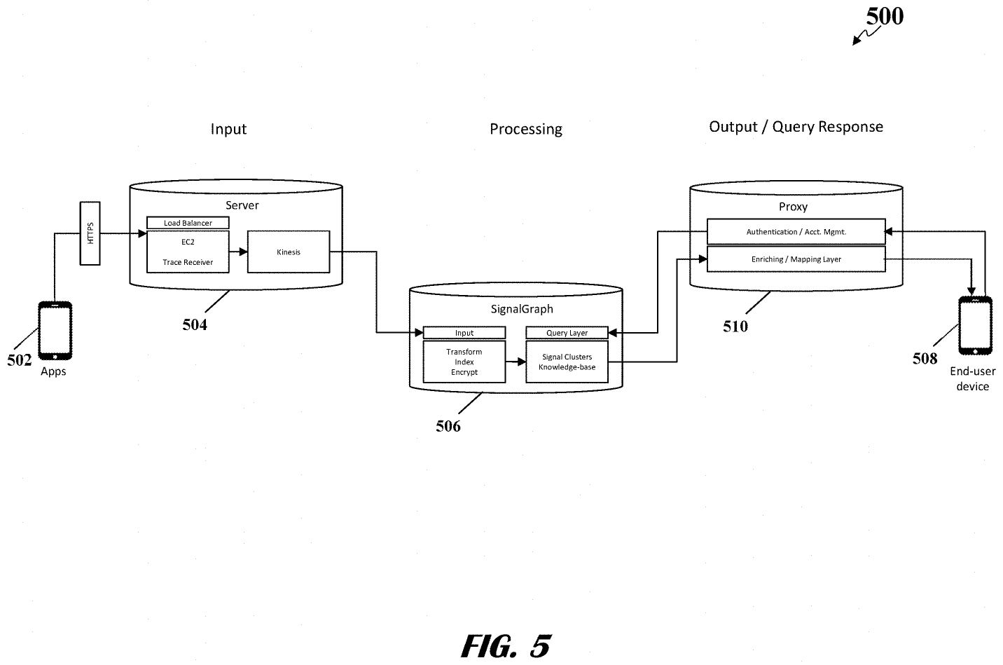

In general, as described in the below passages and specifically in the description of FIG. 6, an employer may operate a human resource server that is configured to deploy (e.g., deliver, send, broadcast) an application and corresponding configuration data (e.g., behavioral rules, geo-fence data, etc.) to the one or more client devices (e.g., a smartphone, a laptop, etc.) that are associated with the respective employees of the organization. Upon receiving an employee's consent (e.g., implied, expressed), the application loads (e.g., installs) onto the employee's client device as a stand-alone application or as a software development kit (SDK) that embeds in and/or interoperates with one or more applications (e.g., an email client, an employee directory, a virtual private network, a browser) that are previously installed on the employee's client device.

Once installed and launched (e.g., executed, run), the application causes the employee's client device to self-configure as an “observer device” that operates (e.g., behaves, functions) according to the configuration data, and sends a dataset (sometimes referred to as, “reporting data”) back to the HR server. The dataset/reporting data may include an observer identifier (which is uniquely associated with the employee's client device) that is generated by the observer device, the employee's name that is associated with the employee's client device, and/or the employee's contact information (e.g., email address, phone number). The HR server stores the reporting data that it receives from the plurality of client devices that are configured as observer devices in a database. The HR server may retrieve the reporting data from the database at a future time to facilitate sending notifications (e.g., alerts, warnings, messages, etc.) to one or more employees of the organization.

The employee's client device operates as an observer device when the employee's client device is near (e.g., within a specific distance) or within a virtual perimeter (sometimes referred to as, “a geo-fence”) defining the employer's physical property (e.g., according to a building layout, property lines, a parcel map, longitude and latitude lines). In some embodiments, the employer may allow the client device (if the employee provides consent) to operate as an observer device even when the client device is outside the virtual perimeter.

A client device that is configured as an observer device is capable of capturing (e.g., monitoring, detecting, observing) and/or analyzing the wireless signals that are emitted by one or more wireless devices (e.g., other client devices operating as an observer device, an IoT device operating as an observer device) that are near (e.g. proximate, adjacent, neighboring) the client device. The employee's client device, as configured as an observer device, determines (e.g., detects, senses, measures, etc.) the signal strength (e.g., signal power in dB or dBm) of the captured wireless signals and/or extracts an identifier (e.g., an observer ID, an SSID) from the captured wireless signal, where the identifier is uniquely associated with the wireless device. The employee's client device then generates a dataset (sometimes referred to herein as, “observation data”) that includes an identifier (e.g., an observer ID, an SSID) of the employee's client device, the identifier of the wireless device that emitted the wireless signal, the corresponding signal strength of the wireless signal, and/or a timestamp (sometimes referred to herein as, “a timepoint”) indicating when the employee's client device captured the wireless signal.

The application executing on the client device causes the client device to periodically push (e.g., send, transmit, deliver) the observation data to an analytic server via a network. That is, the client device sends the observation data to the analytic server regardless of whether the analytic server sends a request for the observation data to be sent. As a result, the analytic server may receive one or more sets of observation data from the one or more client devices that are configured as observer devices. In response to receiving the sets of observation data, the analytic server may store (e.g., maintain) the sets of observation data in a database that is managed and/or controlled by the analytic server.

The analytic server is configured to quantify (or rank) the degree of interaction (e.g., association, contact) that an employee had with other employees based on the wireless signals emitted from their respective client devices. For example, the analytic server executes an application programming interface (API) that is configured to receive calls or requests (e.g., tracing data requests) from an application (e.g., a browser providing access to a web portal) executing on the HR server. The request, which includes an observer identifier to an observer device of a specific employee (e.g., a sick employee) of the organization, instructs the analytic server to generate a dataset (e.g., tracing data) that includes the observer identifiers that identify the other observer devices (i.e., the observer devices associated with the other employees of the organization) that were within a predetermined signal range of the observer device of the specific employee (e.g., sick employee) for any amount of time and/or number of times (i.e., repeat contact).

The analytic server also generates, and includes in the tracing data, a plurality of risk scores that are mapped to the plurality of observer identifiers of the other observer devices, where each risk score (e.g., high, medium, low) indicates the degree of interaction that an employee had with the specific employee (e.g., sick employee).

Thus, the analytic server is able to trace (e.g., determine, detect, discover) the interactions, including the movements and patterns of movements, an employee of an organization may have had with other employees based on analyzing the wireless signals that are emitted from their client device that is operating as an observer device, and in some instances, the wireless signals emitted from non-client devices (e.g., an IoT device, etc.) that are also operating as observer devices. An employer may then use the tracing capability of the analytic server to identify an employee who may have come in contact with a known sick employee, and notify the exposed employee to self-quarantine, thereby preventing and/or mitigating the spread of infectious diseases (e.g., COVID-19) at the workplace.

FIG. 6 is an example network environment for generating risk scores based on wireless signals, according to an embodiment. The network environment 600 may include an analytic server 602 for generating tracing data (e.g., a mapping of observer identifiers to risk scores) associated with an observer device (e.g., a smartphone, a laptop, a Bluetooth beacon, etc.). The network environment 600 may include an observation data database 604a coupled to the analytic server 602 for storing the observation data that is acquired and/or generated by one or more observer devices 606.

The network environment 600 may include an HR server 650 that is controlled and/or managed by an organization 640 (e.g., employer, a business, a non-profit, an academic institution, etc.) and is interconnected with the analytic server 602 via a network 616 (e.g., network 116 in FIG. 1A). The HR server may be located on the property of the organization 640, or in a remote location (e.g., a cloud service).

The network environment 600 may include an HR data database 604b coupled to the HR server 650 for storing HR data (e.g., observer identifiers, employee names, contact information). The HR server 650 may populate the HR data database 604b using any information about an employee that is provided to the organization 640. For example, the HR server 650 may populate the HR data database 604b using the reporting data that an observer device sends to the organization 640. As shown in FIG. 6, the HR server 650 may store the data in the HR database 604b using a data structure that maps (e.g., associates, links) each observer identifier (shown in FIG. 6 as, “Obs. ID”) of an observer device to an employee name (shown in FIG. 6 as, “EE Name”) and contact information (shown in FIG. 6 as, “Contact Info”). For example, the observer identifier of ‘425’ is mapped to the employee name of ‘Bill N’ who may be contacted using ‘email 1’. In some embodiments, the data structure may map each observer identifier to a name for an IoT device (e.g., beacon, smart device), where the IoT device is operating as an observer device.

The HR server 650 may use the HR data that is stored in the HR data database 604b to send notifications to an employee. For example, if the HR server 650 receives a message from a computing device (e.g., analytic server 602, health messaging system 660) indicating that an employee has been exposed to an infectious disease, the HR server 650 can search the HR data database 604b for the contact information that is associated with the employee's name or observer identifier.

The network environment 600 may include a configuration database 604c (shown in FIG. 6 as, “Config. Data Database”) coupled to the HR server 650 for storing configuration data (e.g., an executable application and/or SDK, behavioral rules, geo-fence data, etc.) that defines the behavior for an observer device 606. The HR server 650 may generate the configuration data or receive the configuration data from a third-party (e.g., a software developer).

The network environment 600 may include a health messaging system 660 coupled to the HR server 640 for sending messages (shown in FIG. 6, as “a human health status message”) to notify the organization 640 about the health status (e.g., sick, healthy) of an employee. The health messaging system 660 may be a mobile phone, a landline phone, an email client, a postal service, or any other type of messaging system in which the organization 640 may be notified about an employee's health status. For example, an employee's doctor may call the organization 640 to inform the organization 640 that the employee needs to stay home as a result of being sick. As another example, a person (e.g., a family member, a doctor, another employee) may walk into the HR department of the organization 640 and verbally inform the HR department about the employee's health status.

The network environment 600 may include one or more observer devices 606 (e.g., observer device 106 in FIG. 1A) that are interconnected with the analytic server 602 via the network 616. The network environment 600 may include a Wi-Fi router 608 (e.g., Wi-Fi router 108 in FIG. 1A), a Wi-Fi router 610 (e.g., a Wi-Fi router 110 in FIG. 1A), a BLE transmitter 612 (e.g., BLE transmitter 112 in FIG. 1A), and/or a Bluetooth transmitter 614 (e.g., Bluetooth transmitter 114 in FIG. 1A). Although FIG. 1A shows only a select number of computing devices (e.g., observer devices, Wi-Fi routers, BLE transmitters, Bluetooth transmitters, analytic servers, HR servers, databases, etc.), the network environment 600 may include any number of computing device (in any combination) that are interconnected in any arrangement to facilitate the exchange of data between the computing devices.

As shown in FIG. 6, the organization 640 and/or the HR server 650 may send configuration data (shown in FIG. 6 as, “Config data”) to one or more client devices (e.g., smartphones, laptops, etc.) associated with the employees of the organization 640 via the network 616. The configuration data may cause the one or more client devices to install and launch (e.g., execute, run) an application (or SDK) that configures the client devices as observer devices (e.g., observer devices 606). Once configured, an observer device sends its identifying information (shown in FIG. 6 as, “reporting data”) back to the HR server 650, which stores the reporting data in the HR data database 604b. The reporting data may include an observer identifier, the employee's name that is associated with the employee's observer device, and/or the employee's contact information (e.g., email address, phone number) or other identifier for the employee. The observer devices 606 are also configured to capture the wireless signals emitted from the neighboring wireless devices, generate sets of observation data (shown in FIG. 6 as, “observation data”) based on the wireless signals, and/or transmit the sets of observation data to the analytic server 602.

The analytic server 602 stores the sets of observation data that it receives from the observer devices 606 in the observation data database 604a. As shown in FIG. 6, the analytic server 602 may store the sets of observation data in the observation data database 604a using a data structure that maps an observer identifier of a “first” observer device to the observer identifier of a “second” observer device (shown in FIG. 6 as, “Prox. Obs. ID”) that is proximate to the “first” observer device; a signal strength (shown in FIG. 6 as, “Sig. Str.) that the “first” observer device detects from the wireless signal emitted by the “second” observer device; and a timestamp (shown in FIG. 6 as, “Timestamp) of when the “first” observer device captured the wireless signal. For example, on Jun. 24, 2019 at 19:35:06, an observer device associated with the observer identifier of ‘425’ captured a wireless signal that was emitted from an observer device associated with the observer identifier of ‘325’ and measured a signal strength of the wireless signal as ‘−75 dB’.

The analytic server 602 may use the observation data to determine the movement patterns of an observer device and its degree of interaction (e.g., association, contact) with other observer devices based on the wireless signals emitted from the observer devices. For example, on Jun. 24, 2019 at 19:35:36, the observer device associated with the observer identifier of ‘425’ captured a wireless signal that was emitted from an observer device associated with the observer identifier of ‘325’ and measured a signal strength of the wireless signal as ‘−63 dB’. On Jun. 24, 2019 at 21:00:08, the observer device associated with the observer identifier of ‘425’ captured a wireless signal that was emitted from an observer device associated with the observer identifier of ‘201’ and measured a signal strength of the wireless signal as ‘−85 dB’. Based on this information, the analytic server 602 could determine that the two observer devices have moved closer together and that the two observer devices share a spatial proximity for a duration of at least 30 seconds.

As another example, on Jun. 26, 2019 at 09:02:44, the observer device associated with the observer identifier of ‘201’ captured a wireless signal that was emitted from an observer device associated with the observer identifier of ‘124’ and measured a signal strength of the wireless signal as ‘−75 dB’. On Jun. 26, 2019 at 09:02:50, the observer device associated with the observer identifier of ‘201’ captured a wireless signal that was emitted from an observer device associated with the observer identifier of ‘563’ and measured a signal strength of the wireless signal as ‘−45 dB’. Based on this information, the analytic server 602 could determine that the observer device associated with the observer identifier of ‘563’ is closer to the observer device associated with the observer identifier of ‘201’, as compared to the observer device associated with the observer identifier of ‘124’.

Now that the analytic server 602 has characterized the movement patterns of the observer devices and their degree of interaction, the analytic server 602 may generate and assign a risk score (e.g., high, medium, low) to an observer device indicating that the employee that is associated with the observer device had an exposure risk to an infected employee. An employee associated with a “high” exposure risk (e.g., attended the same meeting in a conference room, sat across from one another at the same lunch table, etc.) indicates that the employee had more interaction with the infected employee, as compared to an employee associated with a “low” exposure risk.

The analytic server 602, in some embodiments, may generate a risk score for a “second” observer device based on the signal strength (i.e., distance between the first and second observer devices) of the wireless signal emitted from the “second” observer device as detected by a “first” observer device. The analytic server 602, in some embodiments, may generate a risk score for a “second” observer device based on a duration during which the “first” observer device detects the wireless signal emitted from the “second” observer device having a signal strength at or above a predetermined value. The predetermined value may be any value between −100 dB and 0 dB. The analytic server 602, in some embodiments, may generate a risk score for a “second” observer device based on the number of times (i.e., repeated contact) in which the “first” observer device detects the wireless signal emitted from the “second.” The analytic server 602, in some embodiments, may generate a risk score based on any combination of distance, duration, and/or repeated contact.

When the organization 640 (or the HR server 650) receives a message (human health status message) from the health message system 660 indicating that an employee has been exposed to an infectious disease, the HR server 650 may send a request (shown in FIG. 6 as, “tracing data request”) to the analytic server 602 for tracing data that is associated with the employee's observer device. The request may include information, such as an observer identifier associated with the employee's observer device (i.e., the employee's client device that is operating as an observer device), an identifier to the employee (e.g., name, contact information, employment title), geo-fence data associated with the organization 640, and/or geographic location data associated with the employee's observer device.

In response to receiving the request, the analytic server 602 may extract the information from the request and retrieve one or more sets of observation data from the observation data database 604a that are associated with the extracted information. For example, the analytic server 602 may retrieve one or more sets of observation data from the observation data database 604a that are associated with the observer identifier of the employee's observer device. In some embodiments, the analytic server 602 may decrease the amount of time needed to search and/or retrieve the observation data from the observation data database 604a by limiting (e.g., filtering) its search to only those directories that were populated with data from observer devices 606 that were in the same geographic location as the employee's observer device. For example, the analytic server 602 would search the directory that stores observation data that was acquired by observer devices 606 that were located in California, if the information extracted from the request indicated that the employee only worked in Los Angeles, Calif.

The analytic server 602 generates a dataset (shown in FIG. 6 as, “tracing data”) that includes a plurality of observer identifiers and a plurality of risk scores, where each risk score is associated with a respective observer identifier. The tracing data identifies the observer devices that are associated with those employees of the organization 640 who were within a predetermined proximity (as defined by a signal strength) of an infected employee (as identified by the observer identifier extracted from the request). In some embodiments, the tracing data only identifies the observer devices that are associated with those employees of the organization 640 who were within a predetermined proximity (as defined by a signal strength) of an infected employee for a predetermined amount of time and/or who entered the predetermined proximity more than a predetermined number of times.

The analytic server 602 transmits the tracing data to the HR server 650. In response to receiving the tracing data, the HR server 650 extracts the observer identifiers from the tracing data and retrieves the contact information associated with each of the observer identifiers from the HR database 604b. For each of the retrieved contact information, the HR server 650 generate a message (shown in FIG. 6 as, “alert data”) and transmits the message to the employee associated with the contact information to notify the employee to self-quarantine

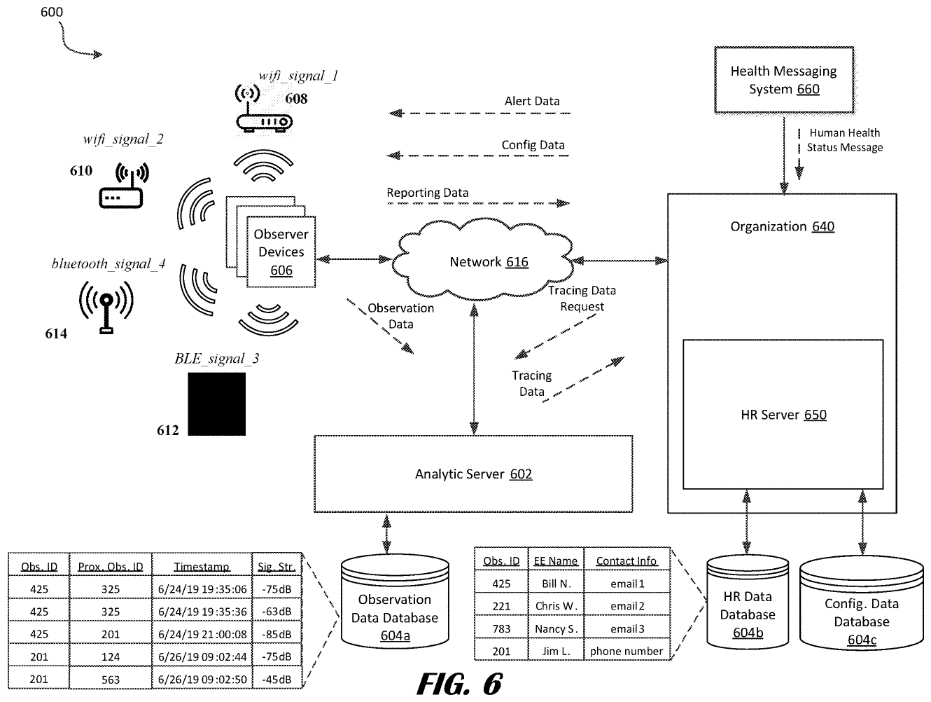

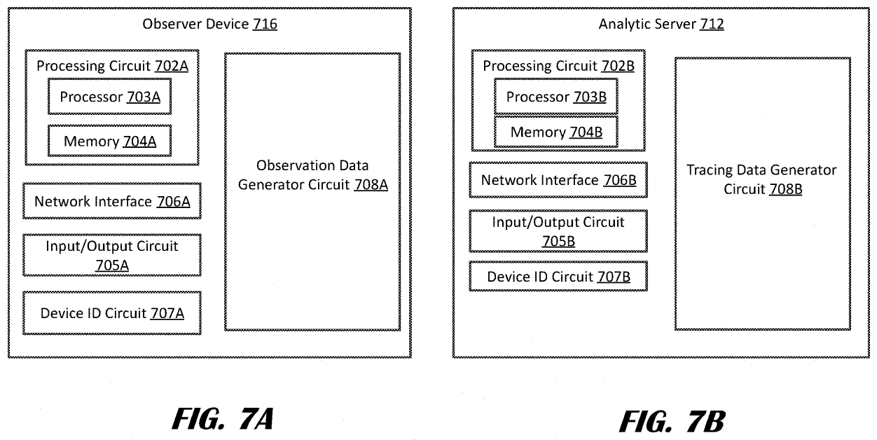

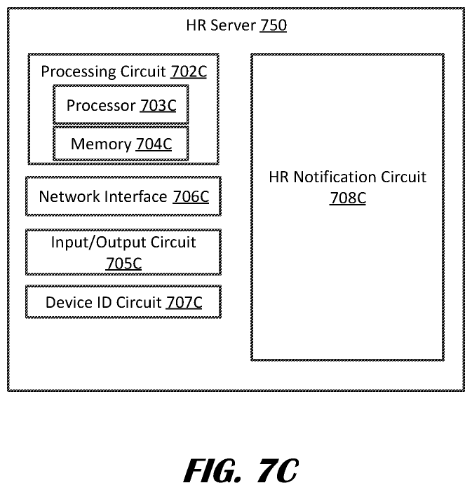

FIG. 7A is a block diagram depicting an example observer device of the network environment in FIG. 6, according to an embodiment. While various circuits, interfaces, and logic with particular functionality are shown, it should be understood that the observer device 606 includes any number of circuits, interfaces, and logic for facilitating the functions described herein. For example, the activities of multiple circuits may be combined as a single circuit and implemented on a single processing circuit (e.g., processing circuit 702A), as additional circuits with additional functionality are included.

The observer device 716 includes a processing circuit 702A composed of one or more processors 703A and a memory 704A. A processor 703A may be implemented as a general-purpose processor, a microprocessor, an Application Specific Integrated Circuit (ASIC), one or more Field Programmable Gate Arrays (FPGAs), a Digital Signal Processor (DSP), a group of processing components, or other suitable electronic processing components. In many embodiments, processor 703A may be a multi-core processor or an array (e.g., one or more) of processors.

The memory 704A (e.g., Random Access Memory (RAM), Read-Only Memory (ROM), Non-volatile RAM (NVRAM), Flash Memory, hard disk storage, optical media, etc.) of processing circuit 702A stores data and/or computer instructions/code for facilitating at least some of the various processes described herein. The memory 704A includes tangible, non-transient volatile memory, or non-volatile memory. The memory 704A stores programming logic (e.g., instructions/code) that, when executed by the processor 703A, controls the operations of the observer device 716. In some embodiments, the processor 703A and the memory 704A form various processing circuits described with respect to the observer device 716. The instructions include code from any suitable computer programming language such as, but not limited to, C, C++, C#, Java, JavaScript, VBScript, Perl, HTML, XML, Python, TCL, and Basic. In some embodiments (referred to as “headless servers”), the observer device 716 may omit the input/output circuit (e.g., input/output circuit 705A), but may communicate with an electronic computing device via a network interface (e.g., network interface 706A).

The observer device 716 includes a network interface 706A configured to establish a communication session with a computing device for sending and receiving data over a network to the computing device. Accordingly, the network interface 706A includes a cellular transceiver (supporting cellular standards), a local wireless network transceiver (supporting 802.11X, ZigBee, Bluetooth, Wi-Fi, or the like), a wired network interface, a combination thereof (e.g., both a cellular transceiver and a Bluetooth transceiver), and/or the like. In some embodiments, the observer device 716 includes a plurality of network interfaces 706A of different types, allowing for connections to a variety of networks, such as local area networks or wide area networks including the Internet, via different sub-networks.

The observer device 716 includes an input/output circuit 705A configured to receive user input from and provide information (e.g., notifications, alerts) to a user of the observer device 716. In this regard, the input/output circuit 705A is structured to exchange data, communications, instructions, etc. with an input/output component of the observer device 716. Accordingly, input/output circuit 605A may be any electronic device that conveys data to a user by generating sensory information (e.g., a visualization on a display, one or more sounds, tactile feedback, etc.) and/or converts received sensory information from a user into electronic signals (e.g., a keyboard, a mouse, a pointing device, a touch screen display, a microphone, etc.). The one or more user interfaces may be internal to the housing of the observer device 716, such as a built-in display, touch screen, microphone, etc., or external to the housing of the observer device 716, such as a monitor connected to the observer device 716, a speaker connected to the observer device 716, etc., according to various embodiments. In some embodiments, the input/output circuit 705A includes communication circuitry for facilitating the exchange of data, values, messages (e.g., alerts sent from HR server 650), and the like between the input/output device and the components of the observer device 716. In some embodiments, the input/output circuit 705A includes machine-readable media for facilitating the exchange of information between the input/output device and the components of the observer device 716. In still another arrangement, the input/output circuit 705A includes any combination of hardware components (e.g., a touchscreen), communication circuitry, and machine-readable media.

The observer device 716 includes a device identification circuit 707A (shown in FIG. 7A as device ID circuit 707A) configured to generate and/or manage a device identifier (e.g., observer ID) associated with the observer device 716. The device identifier may include any type and form of identification used to distinguish the observer device 716 from other computing and/or observer devices. In some embodiments, to preserve privacy, the device identifier may be cryptographically generated, encrypted, or otherwise obfuscated by any circuit of the observer device 716. In some embodiments, the observer device 716 may include the device identifier (e.g., observer ID) in any communication (any of the messages in FIG. 28, e.g., observation data, reporting data, etc.) that the observer device 716 sends to a computing device.

The observer device 716 includes an observation data generator (ODG) circuit 708A that is configured to receive configuration data from the HR server 750. In response to receiving the configuration data, the ODG circuit 708A installs an application or SDK contained within the configuration data that configures the ODG circuit 708A to behave according to the configuration data. The ODG circuit 708A is configured to send reporting data (e.g., observer ID, etc.) back to the HR server 750. The observer devices 716 is configured to listen for (e.g., monitor) and capture wireless signals emitted from the neighboring wireless devices. The observer devices 716 is configured to generate observation data based on the wireless signals. The observer devices 716 is configured to transmit the observation data to the analytic server 602.

The observer device 716 includes a bus (not shown), such as an address/data bus or other communication mechanism for communicating information, which interconnects circuits and/or subsystems of the observer device 716. In some embodiments, the observer device 716 may include one or more of any such circuits and/or subsystems.

In some embodiments, some or all of the circuits of the observer device 716 may be implemented with the processing circuit 702A. For example, any of the observer device 716 may be implemented as a software application stored within the memory 704A and executed by the processor 703A. Accordingly, such arrangement can be implemented with minimal or no additional hardware costs. In some embodiments, any of these above-recited circuits rely on dedicated hardware specifically configured for performing operations of the circuit.

FIG. 7B is a block diagram depicting an example analytic server of the network environment in FIG. 6, according to an embodiment. While various circuits, interfaces, and logic with particular functionality are shown, it should be understood that the analytic server 712 includes any number of circuits, interfaces, and logic for facilitating the functions described herein. For example, the activities of multiple circuits may be combined as a single circuit and implemented on a single processing circuit (e.g., processing circuit 702A), as additional circuits with additional functionality are included.

The analytic server 712 includes a processing circuit 702B composed of one or more processors 703B and a memory 704B. The processing circuit 702B includes identical or nearly identical functionality as processing circuit 702A in FIG. 7A, but with respect to circuits and/or subsystems of the analytic server 712 instead of circuits and/or subsystems of the observer device 716.

The memory 704B of processing circuit 702B stores data and/or computer instructions/code for facilitating at least some of the various processes described herein. The memory 704B includes identical or nearly identical functionality as memory 704A in FIG. 7A, but with respect to circuits and/or subsystems of the analytic server 712 instead of circuits and/or subsystems of the observer device 716.

The analytic server 712 includes a network interface 706B configured to establish a communication session with a computing device for sending and receiving data over a network to the computing device. Accordingly, the network interface 706B includes identical or nearly identical functionality as network interface 706A in FIG. 6A, but with respect to circuits and/or subsystems of analytic server 712 instead of circuits and/or subsystems of the observer device 716.

The analytic server 712 includes an input/output circuit 705B configured to receive user input from and provide information to a user. In this regard, the input/output circuit 705B is structured to exchange data, communications, instructions, etc. with an input/output component of the analytic server 712. The input/output circuit 705B includes identical or nearly identical functionality as input/output circuit 705A in FIG. 7A, but with respect to circuits and/or subsystems of analytic server 712 instead of circuits and/or subsystems of the observer device 606.

The analytic server 712 includes a device identification circuit 707B (shown in FIG. 7B as device ID circuit 707B) configured to generate and/or manage a device identifier associated with the analytic server 712. The device ID circuit 707B includes identical or nearly identical functionality as device ID circuit 707A in FIG. 7A, but with respect to circuits and/or subsystems of analytic server 712 instead of circuits and/or subsystems of the observer device 716.

The analytic server 712 includes a tracing data generator (TDG) circuit 708B that is configured to receive sets of observation data (e.g., a plurality of observer identifiers, a plurality of signal strengths, etc.) from one or more observer devices 716. In response to receiving the sets of observation data, the TDG circuit 708B stores the sets of observation data in a database (e.g., observation data database 604a in FIG. 6). The TDG circuit 708B is configured to maintain, in the database and using the sets of observation data, an association between a plurality of observation identifiers and a plurality of signal strengths, where each signal strength corresponding to a respective wireless signal that was emitted by a respective observer device.

The TDG circuit 708B is configured to receive a request to generate tracing data from the HR server 750. In some embodiments, the request may be received from an application (e.g., a browser providing access to a web portal) executing on the HR server 750, in this instance an application programming interface (API) Layer executing on the TDG circuit 708B may receive the request and pass the request to the TDG circuit 708B. In response to receiving the request, the TDG circuit 708B extracts an observer identifier from the request.

The TDG circuit 708B is configured to generate tracing data by identifying, using the observation data from the database, the one or more observer devices 716 (and their respective observer identifiers) that had a predetermined amount of exposure (or interaction) to the observer device that was identified in the request. Using the observation data from the database, the TDG circuit 708B generates a rank that quantifies the degree of interaction that each of the one or more observers 716 had with the observer device that was identified in the request. The TDG circuit 708B may generate the rank (e.g., high, medium, low) based on whether an observer devices was within a predetermined distance from the observer device (i.e., the observer device that was identified in the request) for a predetermined amount of time and/or a predetermined number of times. The TDG circuit 708B generates the tracing data by including a mapping of the observer identifiers corresponding to the observer devices with an exposure risk and their corresponding ranks. The TDG circuit 708B is configured to transmit the tracing data to the HR server 750.

The TDG 2908 includes a bus (not shown), such as an address/data bus or other communication mechanism for communicating information, which interconnects circuits and/or subsystems of the TDG 2908. In some embodiments, the TDG 2908 may include one or more of any such circuits and/or subsystems.

In some embodiments, some or all of the circuits of the TDG 2908 may be implemented with the processing circuit 702B. For example, any of the TDG 2908 may be implemented as a software application stored within the memory 704B and executed by the processor 703B. Accordingly, such arrangement can be implemented with minimal or no additional hardware costs. In some embodiments, any of these above-recited circuits rely on dedicated hardware specifically configured for performing operations of the circuit.

FIG. 7C is a block diagram depicting an example HR server of the network environment in FIG. 6, according to an embodiment. While various circuits, interfaces, and logic with particular functionality are shown, it should be understood that the HR server 750 includes any number of circuits, interfaces, and logic for facilitating the functions described herein. For example, the activities of multiple circuits may be combined as a single circuit and implemented on a single processing circuit (e.g., processing circuit 702A), as additional circuits with additional functionality are included.

The HR server 750 includes a processing circuit 702C composed of one or more processors 703C and a memory 704C. The processing circuit 702C includes identical or nearly identical functionality as processing circuit 702A in FIG. 7A, but with respect to circuits and/or subsystems of the HR server 750 instead of circuits and/or subsystems of the observer device 716.

The memory 704C of processing circuit 702C stores data and/or computer instructions/code for facilitating at least some of the various processes described herein. The memory 704C includes identical or nearly identical functionality as memory 704A in FIG. 7A, but with respect to circuits and/or subsystems of the HR server 750 instead of circuits and/or subsystems of the observer device 716.

The HR server 750 includes a network interface 706C configured to establish a communication session with a computing device for sending and receiving data over a network to the computing device. Accordingly, the network interface 706C includes identical or nearly identical functionality as network interface 706A in FIG. 6A, but with respect to circuits and/or subsystems of HR server 750 instead of circuits and/or subsystems of the observer device 716.

The HR server 750 includes an input/output circuit 705C configured to receive user input from and provide information to a user. In this regard, the input/output circuit 705C is structured to exchange data, communications, instructions, etc. with an input/output component of the HR server 750. The input/output circuit 705C includes identical or nearly identical functionality as input/output circuit 705A in FIG. 7A, but with respect to circuits and/or subsystems of HR server 750 instead of circuits and/or subsystems of the observer device 716.

The HR server 750 includes a device identification circuit 707C (shown in FIG. 7C as device ID circuit 707C) configured to generate and/or manage a device identifier associated with the HR server 750. The device ID circuit 707C includes identical or nearly identical functionality as device ID circuit 707A in FIG. 7A, but with respect to circuits and/or subsystems of HR server 750 instead of circuits and/or subsystems of the observer device 716.

The HR server 750 includes an HR notification circuit 708C that is configured to receive the consent of an employee of an organization (e.g., organization 640 in FIG. 6) to configure a client device (e.g., observer device 716) that is associated with the employee as an observer device. In response to receiving the consent, the HR notification circuit 708C retrieves configuration data from a database (e.g., configuration data database 604c in FIG. 6) and send the configuration data to the client device (e.g., observer device 716) to cause the client device to operate as an observer device.

The HR notification circuit 708C is configured to receive reporting data (e.g., observer ID, etc.) from the client device (e.g., observer device 716). The HR notification circuit 708C is configured to store the reporting data in a database (e.g., HR data database 604b in FIG. 1A).

The HR notification circuit 708C is configured to receive a message (e.g., a human health status message) from a health messaging system 660, where the message includes an identifier (e.g., an employee name) to an employee and an indication that the employee has been exposed to an infectious disease. In response to receiving the message, the HR notification circuit 708C retrieves an observer device identifier associated with the employee from a database (e.g., HR data database 604b in FIG. 6). The HR notification circuit 708C is configured to send a request (e.g., a tracing data request) to the analytic server 712 for tracing data, where the request includes the observer device identifier associated with the “infected” employee.

The HR notification circuit 708C is configured to receive the tracing data from the analytic server 712. In response to receiving the tracing data, the HR notification circuit 708C extracts the one or more observer identifiers from the tracing data, and retrieves the contact information associated with each of the extracted observer identifiers from the HR database 604b. The HR notification circuit 708C is configured to generate, for each of the retrieved contact information (e.g., email address, etc.), generate a message (e.g., alert data) and transmit the message to the employee associated with the contact information to notify the employee to self-quarantine.

The HR server 750 includes a bus (not shown), such as an address/data bus or other communication mechanism for communicating information, which interconnects circuits and/or subsystems of the HR server 750. In some embodiments, the HR server 750 may include one or more of any such circuits and/or subsystems.

In some embodiments, some or all of the circuits of the HR server 750 may be implemented with the processing circuit 702C. For example, any of the HR server 750 may be implemented as a software application (e.g., a browser providing access to a web portal) stored within the memory 704C and executed by the processor 703C. Accordingly, such arrangement can be implemented with minimal or no additional hardware costs. In some embodiments, any of these above-recited circuits rely on dedicated hardware specifically configured for performing operations of the circuit.

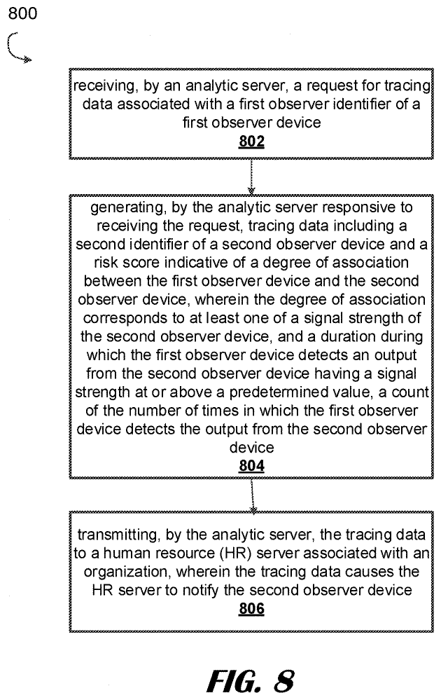

FIG. 8 is a flow diagram depicting a method for generating risk scores based on wireless signals, according to an embodiment. Additional, fewer, or different operations may be performed in the method depending on the particular arrangement. In some embodiments, some or all operations of method 800 may be performed by one or more processors executing on one or more computing devices, systems, or servers. In some embodiments, method 800 may be performed by one or more analytic servers, such as the analytic server 102 in FIG. 1A. In some embodiments, method 800 may be performed by one or more observer devices, such as observer device 106a and/or 106b. Each operation may be re-ordered, added, removed, or repeated.

As shown in FIG. 8, the method 800 includes the operation 802 of receiving, by an analytic server, a request for tracing data associated with a first device identifier (e.g., an observer identifier, an advertisement identifier, etc.) of a first observer device. The method 800 also includes the operation 804 of generating, by the analytic server responsive to receiving the request, tracing data including a second identifier of a second observer device and a risk score (e.g., high, medium, low, etc.) indicative of a degree of association between the first observer device and the second observer device. In some embodiments, the degree of association corresponds to at least one of a signal strength (e.g., expressed in dB or dBm) of the second observer device, and a duration during which the first observer device detects an output from the second observer device having a signal strength at or above a predetermined value, a count of the number of times in which the first observer device detects the output from the second observer device. The method 800 also includes the operation 806 of transmitting, by the analytic server, the tracing data to a human resource (HR) server associated with an organization, wherein the tracing data causes the HR server to notify the second observer device. In some embodiments, the method 800 may include the operation (not shown) of receiving observation data from an observer device (e.g., observer device 106a, 106b in FIG. 1A).

In some embodiments, the observer device may generate the observation data based on a plurality of wireless signals emitted from a plurality of observer devices. In some embodiments, the method 800 may include the operation (not shown) of maintaining, in a database (e.g., database 104 in FIG. 1A, database 604a in FIG. 6) and based on the observation data, an association between a plurality of observer identifiers of the plurality of observer devices and a plurality of signal strengths determined by the observer device. In some embodiments, each signal strength of the plurality of signal strengths corresponding to a respective wireless signal of the plurality of wireless signals that is emitted by a respective observer device of the plurality of observer devices. In some embodiments, the method 800 may include the operation (not shown) of generating, by the analytic server and based on the database, a risk score indicative of a degree of association between a first observer device and a second observer device.

### III. Determining Risk Levels for Users Based on Multiple Types of Inputs for Tracing

Embodiments herein describe systems and methods for determining risk levels for users based on multiple types of inputs for tracing. The systems and methods may determine risk scores using observation data received from observation devices in the system. Certain data fields of the observation data may correspond to risk factors that can be used to determine risk score. For example, a signal strength value can be used as a distance risk factor. Beneficially, some embodiments may use information to determine a risk score in addition or as an alternative to observed signal data. Such additional information can include, for example, information about the other observed devices and information about a user's patterns, among other types of information that may be directly received from or algorithmically derived by other observer devices, servers, or databases of the system.

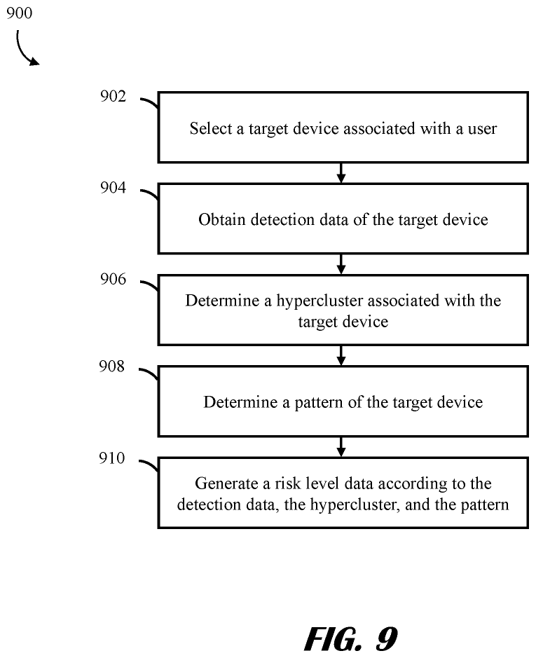

FIG. 9 illustrates a flowchart 900 for determining a risk level of a target device based on detection data of the target device, a hypercluster associated with the target device, and a pattern of movements of the target device, according to an embodiment. In some embodiments, the method shown in the flowchart 900 is performed by a server (e.g., the analytic server). In some embodiments, the method shown in the flowchart 900 is performed by other entities. Other embodiments may comprise additional or alternative operations, or may omit some operations altogether.

At operation 902, the server may select a target device associated with a user to determine a risk level of a risk of exposure of the user to disease. The target device may be an observer device or any electronic device. The server may receive the request to determine the risk level from a client computing device or server, such as an administrator computer or administrator server. The client device may receive information that the user who is operating or associated with the target device may be exposed to disease or may have a high risk of exposure to such disease. The client device may receive from one or more databases mapping information indicating an association between the user and the target device. The client device may refer to the mapping information, and generate a request including the unique identification (e.g., an observer identifier) of the target device associated with the user. In one aspect, the unique identification may identify, but not the user associated with the target device. Hence, the server receiving the request may operate without identifying the user operating the target device. In response to the unique identification included in the request, the server may select the target device associated with the unique identification.

At operation 904, the server may obtain detection data of the target device. The detection data may indicate a history of detecting wireless signals of other electronic devices (e.g., other observer devices). For example, the detection data may indicate an identification of another electronic device detected by the target device, timestamp at which the another electronic device is detected by the target device, and a signal strength (e.g., Bluetooth, Wi-Fi, or other wireless signals) of the wireless signal from the another electronic device detected the target device.

At operation 906, the server may determine a hypercluster associated the target device. In some embodiments, the server determines the hypercluster associated with the target device, as described above with respect to FIGS. 1A-1C and FIG. 2. In one example, the server receives, from a plurality of electronic device (e.g., observer devices), detection data (sometimes referred to as observation data) and aggregated different detection data. According to the aggregated detection data, the server may determine a number of detections of wireless signals (e.g., Bluetooth, Wi-Fi, etc.) among devices, locations of the detections, and overlapping trajectories of locations of the device. Moreover, the server may determine the hypercluster according to the determined number of detections of wireless signals among devices, locations of the detections, and overlapping trajectories of locations of the device, or any combination thereof. The server may store mapping information indicating an association between identifications of the electronic devices (e.g., observer devices) and associated hyperclusters. The server may apply the unique identification of the target device to such mapping and determine or identify an associated hypercluster.

At operation 908, the server may determine a pattern of movements of the target device. In one aspect, the pattern of movements of the target device indicates or corresponds to a pattern of life of the user operating the target device. In some embodiments, the server determines the pattern of movements of the target device, as described above with respect to FIGS. 1A-1C and FIG. 2. In one example, the server obtains a trajectory of movements of the target device at different time periods, and determines the pattern of movements of the target device, according to the trajectory. The movements or locations of the target device may be detected by other electronic devices (e.g., observer devices 106). For example, the server determines that during business days (e.g., Monday through Friday), the target device is generally located at a gym between 8:00 AM to 9:00 AM, at an office between 9:00 AM to 12:00 PM and 1:00 PM to 6:00 PM, and at a cafeteria between 12:00 PM and 1:00 PM. The server may generate and store different patterns of movements of different devices. The server may obtain or retrieve the pattern of movements of the target device according to the unique identification of the target device.

At operation 910, the server generates a risk level data indicating a risk level of the target device, according to the detection data, the hypercluster, and the pattern. In one aspect, the server determines a risk level indicating or corresponding to a risk of exposure of the user to disease, according to the detection data, the hypercluster, and the pattern. Examples of risk level include a high risk, a medium risk, a low risk, etc. The high risk may correspond to, for example, a centers for disease control and prevention (CDC) guideline. In one approach, the server obtains different risk scores of the target device, according to the detection data, the hypercluster, and the pattern. According to the obtained risk scores of the target deice, the server may determine the risk level of the target device. The server may generate the risk level data indicating the determined risk level of the target device, and transmit the risk level data to the requesting device (e.g., client device).

In some embodiments, the server determines a risk score of the target device, according to the detection data. For example, the server may count a number of times the target device detected another electronic device with the signal strength over a threshold power level (e.g., −65 dB). The threshold power level may correspond to a predetermined distance between the target device and another electronic device. The server may count the number for a given time period (e.g., within an hour, a day, or a week). In one example, the server counts, for each of different electronic devices, a corresponding number of times the target device detected the each of the different electronic devices. The server may multiply or scale each of the counted numbers by a corresponding risk score, and obtains the sum or an average of the multiplication results as the first score. For example, assuming that the target device detected a first device, a second device, a third device, 5 times, 4 times, 2 times, respectively, and that each of the first device, the second device, the third device has a risk score of 3, 7, 1, respectively, the server may compute 5×3, 4×7, and 8×1 and obtain a sum 51 of the multiplication results. The server may divide the sum 51 by the number of device (e.g., 3) to obtain an average 17 as the first score of the target device. In some embodiments, the server may apply a decay function to the first score and/or the counted number, in response to determining that the target device did not detect another electronic device for a predetermined time period (e.g., a week or two weeks). By applying the decay function, the server may decrease the first score and/or the counted number by a predetermined amount.

In some embodiments, the server adjusts the risk score or determines a second risk score of the hypercluster. In some embodiments, the server aggregates risk scores of a plurality of devices associated with the hypercluster, and determines the second risk score of the hypercluster, according to the aggregated risk scores. For example, the server may multiply the aggregated risk scores by a corresponding weight to obtain the second risk score of the hypercluster. The server may divide the aggregated risk scores by a number of devices associated with the hypercluster to obtain an average risk score as the second risk score.

In some embodiments, the server adjusts a risk score or determines a third risk score of the target device, according to the pattern of movements of the target device. In some embodiments, the server determines the pattern of movements of the target device, and predicts coexistences of the target device or the user of the target device with one or more other electronic devices (e.g., observer devices) or one or more hyperclusters. In one aspect, a presence of the target device may not have been detected, but the server may predict coexistence of the target device with another device or another hypercluster based on the determined pattern of movements of the target device. For example, the server may determine that despite the presence of the target device is not detected for a particular time period, the target device likely has visited a gym based on the pattern of movements of the target device, and predict exposures of the target device with another electronic device or hyperclusters that visited the gym. The server may assign the third risk score according to the predicted exposures with another electronic device or hyperclusters. For example, the server may compute, for each device or a hypercluster, a multiplication of a number of predicted exposures and a corresponding risk level, and compute a sum the multiplication results. Then, the server may obtain the average of the multiplication results as the third score of the target device.

In one aspect, the server determines a risk level of the target device, according to the adjusted risk score or one or more of the first risk score, the second risk score, and the third risk score. In one approach, the server updates to the adjusted risk score, or to each of the first risk score, the second risk score, and the third risk score, by a corresponding weight, and obtains a sum of the multiplication results. The server may compare the sum against predetermined threshold levels, and determines the risk level according to the comparison. For example, if the sum exceeds a first threshold value, the server may determine that the user of the target device has a high risk of exposure to disease or exposure to another user associated with the disease. For example, if the sum is between the first threshold value and a second threshold value, the server may determine that the target device has a medium risk of exposure to disease or exposure to another user associated with the disease. For example, if the sum is less than the second threshold value, the server may determine that the target device has a low risk of exposure to disease or exposure to another user associated with the disease. In some embodiments, the threshold values are predetermined. In some embodiments, the threshold values are fixed and unchanged. In some embodiments, the threshold values can be dynamically changed. In some embodiments, the threshold values can be set and/or adjusted by the client device.

Advantageously, the risk of exposures of a user operating the target device can be determined despite lack of detections of other devices by the target device. In one example, a risk of exposure of the user to disease may be determined based on a history of the target device operated by the user detecting wireless signals (e.g., Bluetooth or Wi-Fi) of other adjacent or nearby devices. However, the target device may not be able to detect or process wireless signals from a larger number (e.g., 10 or more) of devices in a given space (e.g., a meeting room), for example, due to interferences, or a limited hardware capacity of the target device. By employing the hypercluster to group a number of devices that have a high likelihood of exposures or coexistences among each other, the risk of exposure to disease can be determined despite lack of detection of wireless signals of certain electronic devices. Moreover, by predicting coexistences of the target device or the user of the target device with other electronic devices based on the pattern of movements of the target device, a risk of exposure of the user to disease may be determined, even when the user lost or does not carry the target device. Accordingly, more comprehensive risk levels of the user operating the target device can be determined.

### IV. Generating Risk Tiers from Hyperclusters and Other Data for Tracing Devices

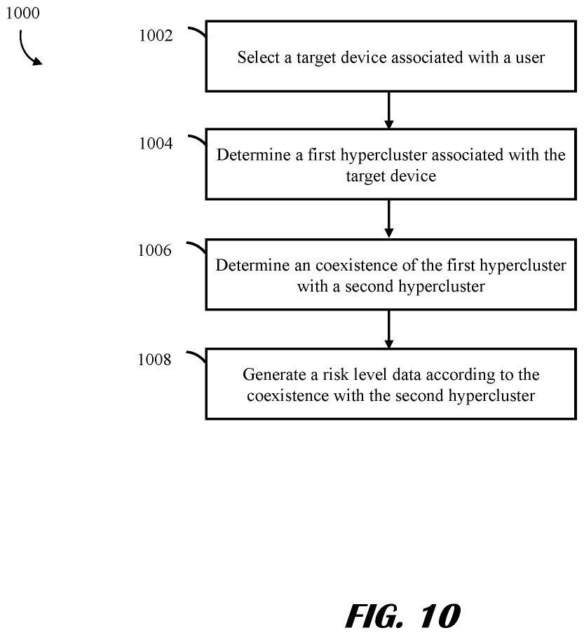

Embodiments herein describe systems and methods for using private hyperclusters (and other data) to trace devices and associated devices with tiers. FIG. 10 illustrates a flowchart 1000 for determining a risk level of a target device based on coexistences of the target device with one or more hyperclusters, according to an embodiment. In some embodiments, the method shown in the flowchart 1000 is performed by a server (e.g., the analytic server). In some embodiments, the method shown in the flowchart 1000 is performed by other entities. Other embodiments may comprise additional or alternative operations, or may omit some operations altogether.

At operation 1002, the server may select a target device associated with a user. The target device may be an observer device or any electronic device. In some embodiments, the server receives a request to determine a risk level of a risk of exposure of the user to disease. The server may receive the request to determine the risk level from a client device, such as an administrator device or an administrator server. The client device may receive information that the user operating or associated with the target device may be exposed to disease or may have a high risk of exposure to such disease. The client device may include or receive from a database mapping information indicating an association between the user and the target device. The client device may refer to the mapping information, and generate a request including the unique identification (e.g., observer identifier) of the target device associated with the user. In one aspect, the unique identification may identify the target device, but not the user associated with the target device. Hence, the server receiving the request may operate without identifying the user operating the target device. In response to the unique identification included in the request, the server may select the target device associated with the unique identification.

At operation 1004, the server may determine a hypercluster associated the target device. In some embodiments, the server determines the hypercluster associated with the target device, as described above with respect to FIGS. 1A-1C and FIG. 2. In one example, the server receives, from a plurality of electronic device (e.g., observer devices), detection data, and aggregated different detection data. According to the aggregated detection data, the server may determine a number of detections of wireless signals (e.g., Bluetooth, Wi-Fi, etc.) among devices, locations of the detections, and overlapping trajectories of locations of the device. Moreover, the server may determine the hypercluster according to the determined number of detections of wireless signals among devices, locations of the detections, and overlapping trajectories of locations of the device, or any combination thereof. The server may store mapping information indicating an association between identifications of the electronic devices (e.g., observer devices) and associated hyperclusters. The server may apply the unique identification of the target device to such mapping and determine or identify an associated hypercluster.

At operation 1006, the server may determine a coexistence of the first hypercluster associated with the target device with a second hypercluster. In one approach, the server determines coexistences of one or more devices of the first hypercluster with one or more devices of the second hypercluster. Furthermore, the server may set or adjust risk level of the target device or the risk levels of the electronic devices in the first hypercluster, according to the determined coexistences. The server may detect or determine that a device (e.g., observer device 106A) in the first hypercluster was adjacent to or nearby another device (e.g., observer device 106B) based on wireless signals (e.g., Bluetooth, Wi-Fi, etc.) detected. For example, in response to a first device detecting a wireless signal from a second device with signal strength exceeding a threshold power level (e.g., −65 dB), the first device may determine that it detected the second device within a predetermined distance corresponding to the threshold power level.

In one approach, the server determines or adjusts a risk score of the target device according to the coexistence of the first hypercluster with the second hypercluster. For example, the server may increase or multiply the risk score of the target device, in response to detecting coexistences of one or more devices associated with the first hypercluster with one or more devices associated with the second hypercluster. In some embodiments, the server increases or multiplies the risk score of the target device by a predetermined amount. In some embodiments, the server increases or multiplies the risk score of the target device according to a risk level of the second hypercluster or risk levels of the detected one or more devices associated second hypercluster.

In one approach, the server determines or adjusts a risk score of the target device according to relative distances of the hyperclusters. Assuming for an example that the target device is associated with a first hypercluster, in response to determining that a user of a first device in the same first hypercluster is exposed to disease, the server may increase or multiply the risk score of the target device by a first amount. For example, in response to determining that a user of a second device in a second hypercluster is exposed to disease and that the second device or a third device in the second hypercluster coexisted with or is exposed to first hypercluster, the server may increase or multiply the risk score of the target device by a second amount less than the first amount. For example, in response to determining that a user of a fourth device in a third hypercluster is exposed to disease and that the fourth device or a fifth device in the third hypercluster is indirectly exposed to the first hypercluster through the second hypercluster, the server may increase or multiply the risk score of the target device by a third amount less than the second amount.

At operation 1008, the server generates a risk level data indicating a risk level of the target device, according to the coexistence with the second hypercluster. In one aspect, the server determines a risk level indicating or corresponding to a risk of exposure of the user to disease, according to the coexistence with the second hypercluster. Examples of risk level include a high risk, a medium risk, and a low risk, etc. The high risk may correspond to, for example, a centers for disease control and prevention (CDC) guideline. The server may generate the risk level data indicating the determined risk level of the target device, and transmit the risk level data to the requesting device (e.g., client device).

In some embodiments, the server compares the risk score of the target device against predetermined threshold levels, and determines the risk level according to the comparison. For example, if the risk score of the target device exceeds a first threshold value, the server may determine that the user of the target device has a high risk of exposure to disease or exposure to another user associated with the disease. For example, if the risk score of the target device is between the first threshold value and a second threshold value, the server may determine that the target device has a medium risk of exposure to disease or exposure to another user associated with the disease. For example, if the risk score of the target device is less than the second threshold value, the server may determine that the target device has a low risk of exposure to disease or exposure to another user associated with the disease. In some embodiments, the threshold values are predetermined. In some embodiments, the threshold values are fixed and unchanged. In some embodiments, the threshold values can be dynamically changed. In some embodiments, the threshold values can be set and/or adjusted by the client device.

Advantageously, the risk of exposures of a user operating the target device can be determined despite lack of detections of other devices by the target device. In one example, a risk of exposure of the user to disease may be determined based on a history of the target device operated by the user detecting wireless signals (e.g., Bluetooth or Wi-Fi) of other adjacent or nearby devices. However, the target device may not be able to detect or process wireless signals from a larger number (e.g., 10 or more) of devices in a given space (e.g., a meeting room), for example, due to interferences, or a limited hardware capacity of the target device. By employing the hypercluster to group a number of devices that have a likelihood of exposures among each other, the risk of exposure to disease can be determined despite lack of detection of wireless signals of certain electronic devices. In addition, the target device may not have directly encountered a first device associated with a different hypercluster, but may have encountered a second device associated with the same hypercluster as the target device. By employing the hypercluster to group a number of devices that have a likelihood of exposure among each other, the risk of indirect exposure to disease can be determined. Accordingly, more comprehensive risk levels of the user operating the target device can be determined.

### V. Generating Risk Tiers by Referencing or Adapting Hypercluster Timing

Embodiments herein describe systems and methods for generating risk tiers by referencing or adapting hypercluster timing. Hyperclusters may be determined according to certain points in time when certain devices were within a certain proximity. The duration of contact between an infected user and other people can be a risk factor for determining a risk score. In some embodiments, it may be beneficial for the system to reference and/or adjust hypercluster time points to correspond with and determine certain levels or tiers of risk to provide a more detailed understanding of the risk.

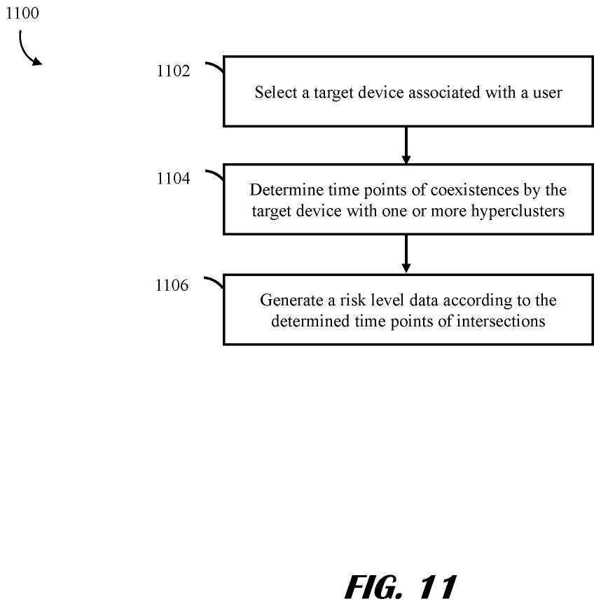

FIG. 11 illustrates a flowchart 1100 for determining a risk level of a target device based on time points of coexistences of target device with one or more hyperclusters, according to an embodiment. In some embodiments, the method shown in the flowchart 1100 is performed by a server (e.g., the analytic server). In some embodiments, the method shown in the flowchart 1100 is performed by other entities. Other embodiments may comprise additional or alternative operations, or may omit some operations altogether.

At operation 1102, the server may select a target device associated with a user. The target device may be an observer device or any electronic device. In some embodiments, the server receives a request to determine a risk level of a risk of exposure of the user to disease. The server may receive the request to determine the risk level from a client device, such as an administrator computer or administrator server. The client device may receive information that the user who is operating or associated with the target device may be exposed to disease or may have a high risk of exposure to such disease. The client device may include or receive from an associated database mapping information indicating an association between the user and the target device. The client device may refer to the mapping information, and generate a request including the unique identification of the target device (e.g., observer identifier) associated with the user. In one aspect, the unique identification may identify the target device, but not the user associated with the target device. Hence, the server (e.g., an analytic server) receiving the request may operate without identifying or knowledge of the user operating the target device. In response to the unique identification included in the request, the server may select the target device associated with the unique identification.

At operation 1104, the server may determine time points of coexistences by the target device with one or more hyperclusters. In one approach, the server determines or adjusts a risk score of the target device according to duration or a length of time periods of coexistences by the target device with one or more hyperclusters. The server may determine the duration or length of time periods of coexistences according to the timestamps.

At operation 1106, the server generates a risk level data indicating a risk level of the target device, according to the time points of coexistences with the one or more hyperclusters. In one aspect, the server determines a risk level indicating or corresponding to a risk of exposure of the user to disease, the time points of coexistences with the one or more hyperclusters. Examples of risk level include a high risk, a medium risk, a low risk, etc. The high risk may correspond to, for example, a centers for disease control and prevention (CDC) guideline. The server may generate the risk level data indicating the determined risk level of the target device, and transmit the risk level data to the requesting device (e.g., client device).

In some embodiments, the server compares the risk score of the target device against predetermined threshold levels, and determines the risk level according to the comparison. For example, if the risk score of the target device exceeds a first threshold value, the server may determine that the user of the target device has a high risk of exposure to disease or exposure to another user associated with the disease. For example, if the risk score of the target device is between the first threshold value and a second threshold value, the server may determine that the target device has a medium risk of exposure to disease or exposure to another user associated with the disease. For example, if the risk score of the target device is less than the second threshold value, the server may determine that the target device has a low risk of exposure to disease or exposure to another user associated with the disease. In some embodiments, the threshold values are predetermined. In some embodiments, the threshold values are fixed and unchanged. In some embodiments, the threshold values can be dynamically changed. In some embodiments, the threshold values can be set and/or adjusted by the client device.

Advantageously, the risk of exposures of a user operating the target device can be determined according to the length of time period or duration of coexistences. In one aspect, a risk of transmission of disease from one person to another may increase linearly or non-linearly (e.g., step function or exponential function). By adjusting a risk score according to the length of time period or duration of coexistences with another device, the risk level of the user of the target device being exposed to disease can be determined in an accurate manner.

### VI. Single-Use Observation Devices (Virtual Observer and Virtual Observations)

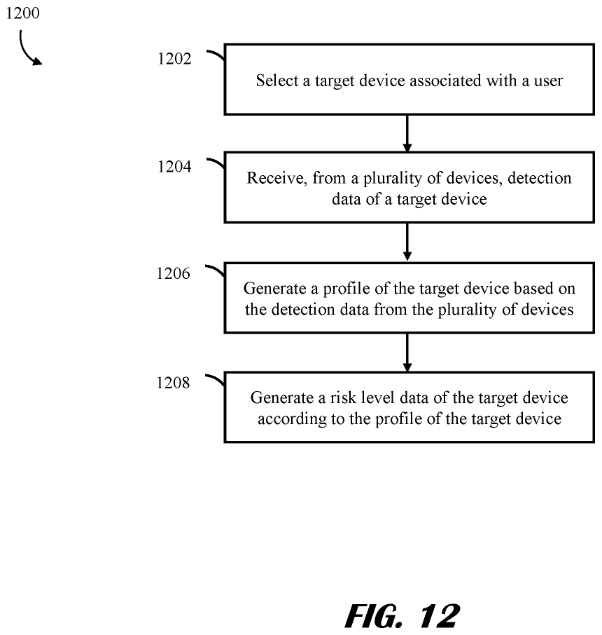

Embodiments herein describe systems and methods for single-use observation devices (virtual observer and virtual observations). FIG. 12 illustrates a flowchart 1200 for determining a risk level of a virtual observer device, according to an embodiment. In some embodiments, the method shown in the flowchart 1200 is performed by a server (e.g., the analytic server). In some embodiments, the method shown in the flowchart 1200 is performed by other entities. Other embodiments may comprise additional or alternative operations, or may omit some operations altogether.

At operation 1202, the server may select a target device associated with a user. The target device may be, for example, a beacon device. The target device may be configured to transmit a wireless signal. The target device may lack a wireless receiver, and may not receive any wireless signal from other devices (e.g., observer devices). In some embodiments, the server receives a request to determine a risk level of a risk of exposure of the user to disease. The server may receive the request to determine the risk level from a client device, such as an administrator computer or administrator server. The client device may receive information that the user operating or associated with the target device may be exposed to disease or may have a high risk of exposure to such disease. The client device may include or receive from a database mapping information indicating an association between the user and the target device. The client device may refer to the mapping information, and generate a request including the unique identification (e.g., observer identifier) of the target device associated with the user. In one aspect, the unique identification may identify the target device, but not the user associated with the target device. Hence, the server receiving the request may operate without identifying the user operating the target device. In response to the unique identification included in the request, the server may select the target device associated with the unique identification.

At operation 1204, the server receives, from each of a plurality of devices (e.g., observer devices), detection data of the target device. The detection data may indicate a history of detecting a wireless signal from the target device by an observer device. For example, the detection data may indicate an identification of the observer device that detected the wireless signal from the target device, timestamp at which the observer device detected the target device, a signal strength (e.g., Bluetooth, Wi-Fi, or other wireless signals) of the wireless signal from the target device detected by the observer device.

At operation 1206, the server generates a profile of the target device based on the detection data from the plurality of devices (e.g., observer devices). In one aspect, the server generates the profile of the target device, such that the target device can be processed as other observer devise. Example profile of the target device includes, locations of the target device, a trajectory of movements of the target device, etc. The server may determine a trajectory of movements of the target device, according to the locations of the plurality of devices, timestamps at which the target device is detected, and signal strengths of the wireless signal from the target device detected by the plurality of devices. For example, if two or more devices detected a wireless signal from the target device at a particular time, the server may compare the signal strengths detected by the two or more devices, and determine that the target device is located closer to a device with the highest signal strength than other devices at the particular time. The server may connect the detected locations at different timestamps to determine a trajectory of movements of the target device.

In one approach, the server determines or adjusts a risk score of the target device according to the profile of the target device. The server may compare the trajectory of movements of the target device with trajectories of movements with other devices (e.g., observer devices 106) or one or more hyperclusters. The server may determine a number of overlaps in the trajectories, and the duration of the overlaps. The server may determine or assign a risk score of the target device, according to the determined number of overlaps and/or duration of the overlaps. For example, the server may increase the risk score of the target device, as the number of overlaps and/or the duration of the overlaps increase.

At operation 1208, the server generates a risk level data indicating a risk level of the target device, according to the profile of the target device. In one aspect, the server determines a risk level indicating or corresponding to a risk of exposure of the user to disease, the time points of coexistences with the one or more hyperclusters. Examples of risk level include a high risk, a medium risk, a low risk, etc. The high risk may correspond to, for example, a centers for disease control and prevention (CDC) guideline. The server may generate the risk level data indicating the determined risk level of the target device, and transmit the risk level data to the requesting device (e.g., client device).

In some embodiments, the server compares the risk score of the target device against predetermined threshold levels, and determines the risk level according to the comparison. For example, if the risk score of the target device exceeds a first threshold value, the server may determine that the user of the target device has a high risk of exposure to disease or exposure to another user associated with the disease. For example, if the risk score of the target device is between the first threshold value and a second threshold value, the server may determine that the target device has a medium risk of exposure to disease or exposure to another user associated with the disease. For example, if the risk score of the target device is less than the second threshold value, the server may determine that the target device has a low risk of exposure to disease or exposure to another user associated with the disease. In some embodiments, the threshold values are predetermined. In some embodiments, the threshold values are fixed and unchanged. In some embodiments, the threshold values can be dynamically changed. In some embodiments, the threshold values can be set and/or adjusted by the client device.

In some embodiments, the server generates a risk level data indicating a risk level of another device (e.g., observer device) or a hypercluster, according to the risk level of the target device. In one approach, the server determines a number of overlaps in trajectories of movements of (i) the target device and (ii) another device or other device in a hypercluster associated with the another device, and/or durations of the overlaps. The server may determine or adjust the risk level of another device or the hypercluster, according to the number of overlaps in trajectories, and/or durations of the overlaps. For example, in response to determining that a user operating the target device is exposed to disease or has a high risk of exposure to the disease, the server may determine that another device or the hypercluster sharing the same or partially overlapping trajectories of movements has a high risk of exposure to such disease.

Advantageously, the server can generate a virtual observer device for a device unable to receive a wireless signal, and determine a risk level of a user operating such device. Moreover, the server can determine risk levels of users of other devices (e.g., observer devices) due to the virtual observer device. In one example, a visitor may not have an observer device configured to communicate or operate with the server (e.g., analytic server). In another example, a user of an observer device may have forgotten to carry the observer device. In above examples, a beacon device can be configured as the virtual observer device based on detection data from other observer devices, and allow determination of the risk level of the user operating the virtual observer device. Furthermore, the beacon device may allow determination of the risk level of users operating other observer devices due to the virtual observer device. In some embodiments, the virtual observer device may be added to a hypercluster, and the risk level of the virtual observer device or other observer devices can be determined according to the hypercluster as disclosed herein.

### VII. Generating a Risk Score Based on Electronic Labels

Embodiments herein describe systems and methods for generating a risk score based on electronic labels. Electronic devices, especially wireless communications devices, are ubiquitous. It is however difficult to determine proximal relationships between these devices in a world where their locations may not be known and/or are continuously changing. Determining device relationships can reveal groups of similar devices and describe properties of different classes of physical spaces (e.g., hotels, restaurants, etc.), in a similar fashion to systems such as recommendation and classification engines. As mentioned, embodiments described herein may capture and analyze observation data received from various types of observation devices, such as, for example, communication devices (e.g., smartphones), and various other electronic devices (e.g., wireless headphones) or smart devices, sometimes referred to as “internet of things” (“IoT”) devices (e.g., ibeacons, smart appliances), having wireless communications capabilities. To mitigate against unwanted outcomes resulting from stray observations or malicious interlopers, devices associated with the system may be enrolled or otherwise known and trusted by the system, as confirmed according to unique observer identifiers. By virtue of having known observer identifiers being broadcasted and received, the observation data is already validated since the observation data is only associated with enrolled, known devices. In some embodiments, the data transmitted and observed between devices may further include a semantic identifier, wireless name, or other type of label data that identifies a participating and trusted observer device according to an another data field in addition to or as an alternative to an observer identifier. Additionally or alternatively, even though data from observer devices may be trusted as a function of the device enrollment, the observation data for a user's personal device is only as reliable as the user's habit of having the device on them as the user interacts with others. The server may perform various machine-learning algorithms on observation data received from a particular observation device to determine a pattern of behavior for the user. Advantageously, the analytic server can use this pattern data to backfill gaps in the observation data associated with the user's movements and interactions. The analytic server can use this pattern data for additional or alternative benefits and operations as well.

For example, once a server has created groups of electronic devices, the server may perform data analytics based on the various types of data the server receives from devices of the groups. But because groupings could be determined based on the proximal relationship of the devices and not based on any other characteristics about the devices or their environment, a server may not be able to infer any information about the groups and treat the data from each group equally when performing analytics.

In an example embodiment, the systems and methods described herein may use groupings of signals to identify different proximal groupings of signals with which a person was associated during a time period (e.g., a day, week, two weeks, etc.). The systems and methods may leverage data gathered from the proximal groupings of signals (sometimes embodied in “hypercluster” data structures) to generate risk scores for individuals indicating a likelihood that an individual is infected (and in some cases a degree of infection) with a disease such as COVID-19. To do so, the system may generate sets of label data and perform analytics on the label data. Labels of the labeled datasets may be associated with characteristics of the groups of signals with which they are associated. For example, the labels may be associated with a physical location of the groups of devices. Observation identifiers or labels may be associated with a corresponding weight for determining an infection risk score. The systems and methods may use the weights associated with one or more devices or the groups of devices with which an observer device has come into contact during the time period to perform analytics to determine a risk score indicating a likelihood that the user associated with the observer device has a particular disease.

A system utilizing the techniques described herein would be able to generate and use trusted and accurate observation datasets to generate such risk scores for individuals based on the labels associated with the respective group of devices. The system may do so by excluding any data that was generated by a potentially untrustworthy or unknown device or group of devices as may be determined by a label or the lack of a label for a particular group of devices. Systems not implementing the techniques described herein may treat data from each group of devices the same, causing the system to generate inaccurate datasets to use as input and potentially produce faulty result such as improper risk scores for diseases.

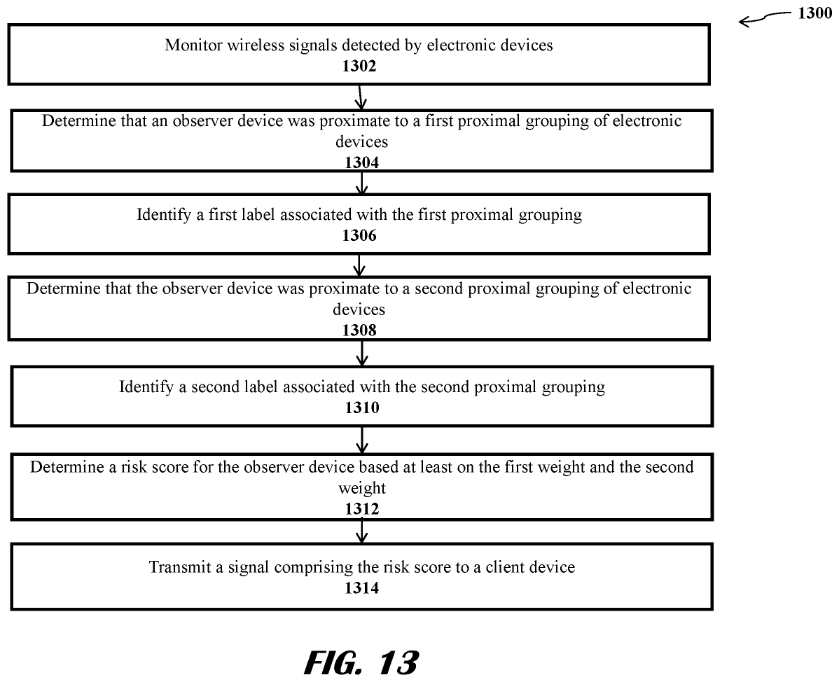

FIG. 13 shows a flow diagram 1300 of a method of generating a risk score based on electronic labels, according to an illustrative embodiment. Other embodiments may comprise additional or alternative steps, or may omit some steps altogether. Although multiple computing systems and databases can implement one or more steps of the method, this description details, for brevity, an analytic server implementing the various steps of the method. In the illustrative method, the analytics identifies weights for locations of an observer device when generating an infection risk score.

At step 1302, the analytic server may monitor wireless signals detected by electronic devices, which include observer devices. The analytic server may monitor the wireless signals using the techniques and methods as discussed herein.

At step 1304, the analytic server may determine that an observer device was proximate to a first proximal grouping of electronic devices (e.g., a first hypercluster). The analytic server may do so based on observation data of the plurality of observer devices. The observation data may comprise wireless signals of the observer device and other wireless signals of the surrounding environment. The analytic server may determine the observer device was proximate to the first hypercluster at the first time point based on one or more signals that identify the observer devices and that are associated with the first hypercluster.

Wireless signals that observer devices detect and transmit to the analytic server may include time points. A time point may be the time that an observer device detected a wireless signal from another device of a hypercluster. In some embodiments, the analytic server determines that the other wireless signals of the surrounding environment are associated with the observer device based on the signals having time points that are the same as or similar to (e.g., within a range or threshold) time points of the wireless signal that the observer device detects. For example, as discussed above, the analytic server may build hyperclusters based on the monitoring of the plurality of electronic devices. Based on observations from the plurality of observer devices, the analytic server may determine the signal context, such as the hypercluster, of the observer device at a given time point by determining the hypercluster of the observer devices that detect wireless signals from the observer device. For example, the analytic server may query a list of detected wireless signals from the observer devices and determine the corresponding hypercluster. The analytic server may determine the hypercluster with which the observer device was associated (e.g., that the observer device was proximate to the hypercluster) based on the observer device detecting and transmitting signals between the same observer devices of the respective hypercluster.

The hypercluster may provide information on the signal environment of the observer device, and further provide information on the physical environment. Because a hypercluster may be a set of signals that have been observed together within a number of observations, a given hypercluster may represent a set of devices that remain in physical proximity over time. In other words, a hypercluster may correspond to a physical location. The analytic server may determine the physical location based on the hypercluster. For example, the analytic server may retrieve data from the database storing the hyperclusters and the corresponding locations.

At step 1306, the analytic server may identify a first label that is associated with the first proximal grouping. A label may be a flag or setting that is stored in the database and/or that the analytic server receives in a detected signal. A label may be a string of characters. A label may be an identifier of a particular hypercluster. In some embodiments, a label may be an indicator of a type of a hypercluster. A type of a hypercluster may be associated with a location (e.g., a type of room such as a kitchen, a gym, a personal office, a hallway, etc.) of a hypercluster within a building or on a premises including a building and grounds around the building, types of activities (e.g., eating, exercising, working, singing, yelling, whispering, etc.) that are performed in a location of a hypercluster, the types of electronic devices of the hypercluster (e.g., mobile phones, headphones, televisions, laptops, LTE Beacons, Bluetooth Beacons, Wi-Fi routers, etc.), etc. In some embodiments, labels may be associated with a combination of the location of the hypercluster and the types of the devices that are associated with the hypercluster. For example, a label may be “gym—headphones.” Such a label may indicate that the hypercluster is associated with a location within a gym and that at least one of the devices associated with the hypercluster is a set of headphones. The labels may be associated with any permutation of devices and/or locations. Such labels may be associated with varying weights and/or other values that the analytic server may use to analyze observation data that it collects, as described herein. In some embodiments, a label may act as a key for the analytic server to identify the hypercluster that is associated with data of a detected signal from the database and consequently the weight associated with the hypercluster and/or label. The analytic server may store associations between the hyperclusters and such labels in a database.

In some embodiments, observer devices of hyperclusters may transmit labels to the analytic server with the detected wireless signals from devices within the hypercluster with which they are associated. Observer devices may include such labels in the header or body of data packets that include data of the detected wireless signals that the observer devices transmit to the analytic server. The observer devices may be configured (e.g., via an input) to transmit such labels to the analytic server. For example, a BLE beacon in a kitchen may be configured to transmit a signal with a label indicating that the BLE beacon is associated with a kitchen label along with any detected wireless signals that the BLE beacon transmits to the analytic server. The analytic server may receive such signals and store data from the wireless signal in the database along with an association between the kitchen label and the detected wireless signals. In another example, a BLE beacon may be configured to transmit a signal with a label indicating that the BLE beacon is in the hallway. The analytic server may store associations between the label and the wireless signals that the BLE beacon provides in the database so the analytic server may identify the labels and characteristics of the labels (e.g., weights) when analyzing the data of the wireless signals.

In some embodiments, the analytic server may only use data from labeled hyperclusters to perform analyses or may only store data from labeled hyperclusters. For example, the analytic server may perform an analysis to perform a trace of the hyperclusters with which a particular observer device came into contact during a time period, which may be specified by an administrator in a query for the trace. In performing the analysis, the analytic server may exclude or disregard any data that the analytic server received from an unlabeled hypercluster. In some embodiments, the analytic server may generate a dataset including data of approximate associations between the observer device and observer devices of a hypercluster (e.g., instances in which the observer devices exchanged signals with the respective hypercluster and/or the observer devices forwarded the signals to the analytic server). The analytic server may perform the analysis on the generated dataset. Advantageously, by only using labeled data to perform an analysis, the analytic server may only use data from known or verified hyperclusters. For example, the analytic server can avoid using data from spoofers attempting to spoof other devices, or other non-trusted devices such as devices that are controlled by other entities (e.g., a company next door that shares a wall) that are not associated with the entity for which the analytic server is performing an analysis.

In some embodiments, a label indicates whether a hypercluster and/or data associated with the hypercluster is valid and/or whether the analytic server will process or store data that it receives that is associated with the hypercluster. For example, a label may be a binary 0 (zero). A label of 0 may indicate that a hypercluster is “invalid” or “not trusted” and consequently that the analytic server will not use the data that it receives from the hypercluster when performing an analysis. In some embodiments, the analytic server may discard or delete from memory any data that it receives from the hypercluster with a label of 0. Similar to above, by only storing and/or processing data from labeled hyperclusters, the analytic server can avoid processing data that may be inaccurate or even fraudulent because it comes from unknown or untrusted sources. A label of another string may indicate that the hypercluster is valid and consequently that the analytic server may use data it receives from the hypercluster when performing an analysis (e.g., an analysis to determine if a particular observer device has been proximal to another observer device at a same or a similar time point, an analysis to determine if the observer device has been proximal to a hypercluster, an analysis to determine which hyperclusters the observer device has been a part of during a time period, etc.) and/or to otherwise store such data.

In some embodiments, an administrator may associate instances of hyperclusters stored in the database with labels. An administrator may do so by inputting labels into a user interface to label instances of hyperclusters within the database with labels indicating the respective types of the hyperclusters. For example, an administrator may, via a user interface displayed on a client device being accessed by the administrator, view a list of hyperclusters that have been instantiated in the database. In some cases, the user interface may additionally display the electronic devices that are associated with the respective hyperclusters. In some embodiments, the analytic server may generate the user interface. For one or more of the hyperclusters, the administrator may select the hypercluster and/or input a label to associate with the particular hypercluster. The analytic server may receive the input and update the database by storing an association between the hypercluster and the label. For example, the analytic server may receive an input from a client device operated by an administrator indicating that a particular hypercluster is associated with the label “gym.” The analytic server may identify the gym label and the hypercluster associated with the gym label and store an association between the gym label and the hypercluster in the database. Any data that the analytic server receives that is associated with the gym hypercluster may be associated with the gym label within the database. Consequently, the analytic server can maintain the database of hyperclusters and labels that are associated with the hyperclusters to analyze observation data.

In some embodiments, the analytic server automatically determines a label for a hypercluster based on the devices that are associated with the respective hypercluster. The analytic server may store associations between labels and the observer devices in the database. The analytic server may use the stored associations between the labels and the observer devices to identify the labels to associate with the respective hyperclusters. To do so, the analytic server may identify the devices that are associated with a hypercluster and determine if any labels are associated with the devices. The analytic server may make such a determination by scanning the database to determine if it has any stored associations between the devices and labels. Responsive to identifying a stored association between an observer device of a hypercluster and a label (e.g., an identifier of the observer device and the label), the analytic server may retrieve the label from the database and determine the retrieved label is associated with the hypercluster. For example, the analytic server may generate a hypercluster that is associated with a BLE beacon in a kitchen, a first observer device, and a second observer device. The analytic server may scan the database to determine if any of the BLE beacon, the first observer device, or the second observer device are associated with a label. The analytic server may identify a label associated with the BLE Beacon with the string “kitchen” based on the scanning. Accordingly, the analytic server may identify “kitchen” as the label that is associated with the hypercluster that is associated with the BLE Beacon, the first observer device, and the second observer device.

Advantageously, by automatically associating hyperclusters with labels based on labels associated with devices, the analytic server may determine labels for newly generated hyperclusters and generate data for the analytic server to use to determine risk scores without any user input. In some instances, labels associated with individual observer devices may be manually entered by an administrator. Consequently, the analytic server can automatically label hyperclusters using labeled data from a trusted source (e.g., the administrator), inhibiting malicious parties from impacting the automatic labeling and thereby increasing the accuracy of such labeling.

In some embodiments, the analytic server may automatically determine labels for hyperclusters based on identifiers (e.g., SSIDs, MAC Addresses, and/or universal unique identifiers) of one or more electronic devices of the respective hypercluster. In some embodiments, the analytic server uses natural language processing techniques such as those discussed herein to determine the semantic resolution of an SSID and consequently a location within a respective building. For example, the analytic server may analyze identifiers of one or more devices of a hypercluster and determine a label for the hypercluster based on the identifiers. For instance, the analytic server may identify the word “gym” in an identifier for a device of a hypercluster. The analytic server may compare the word gym with a list of labels in the database and identify a match. Accordingly, the analytic server may label the hypercluster of the device with the label, gym. In some instances, a label may be associated with multiple words and/or phrases. Responsive to the analytic server identifying a word or phrase from an identifier that matches one of the words or phrases that are associated with the label, the analytic server may select the label associated with the matching word or phrase as the label to associate with the hypercluster of the device. The analytic server may store, in the database, an association between the label and the hypercluster with which the label is associated. The analytic server may use any natural language processing techniques to determine labels for hyperclusters based on the identifiers of the devices of the respective hyperclusters.

In some embodiments, the analytic server may determine the label to associate with a hypercluster based on device labels that are associated with the observer device of the hypercluster. The database may store a series of labels that may each be associated with one or more combinations of device labels. For example, a “gym-headphone” label may be associated with the labels “gym” and “headphones.” The analytic server may identify the device labels from identifiers of observer devices of a hypercluster and compare the device labels to the database to determine if a label matches the identified device labels of the hypercluster. Continuing with the example above, the analytic server may determine a BLE beacon is a part of a hypercluster and identify a “gym” label that is associated with an identifier of the BLE beacon from the database. The analytic server may also determine a set of headphones is a part of the same hypercluster as the BLE beacon and identify a “headphone” label that is associated with an identifier of the set of headphones from the database. The analytic server may compare the combination of the gym label and the headphone label to the database, determine that the combination matches or is otherwise associated with the “gym-headphone” label in the database, and determine that the gym-headphone label is associated with the hypercluster of the BLE beacon and the set of headphones. Any combination of one or more device labels may match or otherwise be associated with the labels in the database. Accordingly, the analytic server may take one or more device labels into account when determining labels for hyperclusters.

The analytic server may store a first association between the first hypercluster and the first label in a database. The first association may be an instance of a tag or setting that the analytic server stores in the database. The analytic server may determine the first label that is associated with the first hypercluster using any one of the methods described above. In some embodiments, the analytic server may determine the first label that is associated with the first hypercluster by identifying a stored association between the first hypercluster and the first label in the database. In some embodiments, the first association is the first label. Upon processing observation data associated with the first hypercluster, the analytic server may identify the first label associated with the first hypercluster from the stored first association.

In some embodiments, the labels are associated with weights. For instance, the first label may be associated with a first weight. As will be described below, the analytic server may use the weights of labels to determine a risk score for the observer device. Weights may range from 0-1, 1-10, 1-100, or any range. The weights may be associated with the respective location of the hypercluster, the actions that are performed in the location, the number of people that are generally around the location, etc. For example, the kitchen may be associated with a high weight because of the number of surfaces in the kitchen that could carry bacteria or viruses while a conference room may have a low risk score because of low through traffic of people.

In some embodiments, weights associated with labels are input by an administrator at a client device. For example, via a user interface, an administrator may input a weight of 0.8 for the gym label and a weight of 0.2 for an outside garden. The analytic server may receive the weights and store associations between the weights and the labels and/or hyperclusters associated with the labels in the database.

In some embodiments, labels may be associated with weights that are particular to a specific disease. For example, a label may be associated with a first weight for COVID-19 and a second weight for allergies or the flu. When inputting weights for various labels, an administrator may identify the disease that is associated with the particular weight. In some cases, the administrator may input multiple weights for a particular label, each weight associated with a different disease. Advantageously, by using different weights for different diseases, an administrator may control the data that the analytic server uses to determine accurate risk scores for a disease without assuming every disease has the same risk factors.

At step 1308, the analytic server may determine that the observer device was proximate to a second proximal grouping of electronic devices (e.g., second hypercluster). One or more observer devices of the second hypercluster may transmit data to the analytic server indicating that the observer device is proximate to the second hypercluster. For example, two of the observer devices of the second hypercluster may transmit signals to the analytic server that include an identifier of the observer device, indicating that the devices are within a proximate range that enables them to exchange signals. In some cases, the signals may include a second time point indicating when they detected the signal from the observer device. Based on receiving such signals, the analytic server may determine that the observer device was proximate to the second hypercluster at the second time point.

At step 1310, the analytic server may identify a second label that is associated with the second hypercluster. The analytic server may identify the second label that is associated with the second hypercluster using the same methods described for the first hypercluster. For example, the analytic server may identify a label in data that the analytic server receives from an observer device of the second hypercluster. The analytic server may identify the label and store an association between the label and the data that the analytic server receives that is associated with the label in the database. The second label may be associated with a second weight. Accordingly, upon processing observation data associated with the second hypercluster, the analytic server may identify the second label associated with the data and, in some cases, identify the second weight associated with the second label from the database.

At step 1312, the analytic server may determine a risk score for the observer device based on at least the first weight and the second weight. In some embodiments, the analytic server may determine the risk score responsive to receiving a query from a client device. The query may include a time frame for the analytic server to process observation data. Upon receiving the query, the analytic server may retrieve, from the database, the observation data associated with time points within the given time frame and determine the risk scores for observer devices based on the retrieved observation data. For example, for the observer device, the analytic server may identify each instance that the observer device was proximate to hyperclusters within a time period of a query based on observation data associated with the observer device. The analytic server may determine that the observer device was proximate to the first hypercluster and the second hypercluster at the first time point and the second time point, respectively, both of which may be associated with times within the time period of the query. The analytic server may identify the first label of the first hypercluster and the second label of the second hypercluster, identify the first weight associated with the first label and the second weight associated with the second label, and determine a risk score for the observer device based on the first and second weight.

The analytic server may determine risk scores for observer devices based on weights by applying one or more algorithms to a dataset. As described herein, weights are values that may be stored in a database of the analytic server or a database from which the analytic server can retrieve the value. The analytic server may use the weight in combination with risk factors for a particular infectious disease to determine a risk score for a user via their observer device. Examples of risk factors may be a length of time that the observer device was at a location (e.g., proximate to a hypercluster and/or RSSI values for the RSSI values of the respective observation data. The risk score may indicate a likelihood that the user has the particular infectious disease.

In some embodiments, when determining the risk score for a person or an observer device, the analytic server may generate datasets indicating the proximate associations and the weights of the proximate associations. When generating the datasets, the analytic server may disregard or discard any data that is not associated with a labeled hypercluster or that is associated with an “invalid” or “not trusted” (e.g., a “0”) label. In some embodiments, the analytic server may generate the datasets and remove any data that is associated with an invalid or a not trusted label. Accordingly, the analytic server may generate trusted datasets and avoid using data that was generated by devices that produce inaccurate data.

Further, because the analytic server includes the weights in the generated datasets, the analytic server may create a robust dataset that takes into account the environmental factors that may be associated with specific diseases. For example, a hypercluster labeled with a gym label may be weighted higher when associated with an infectious disease such as COVID-19 because of the yelling and the contact that normally occurs when a person is in a gym. The gym label may have a low or average weighting for other diseases such as allergies because these factors do not affect a person's risk for experiencing allergies. The opposite may be true for a garden in which a weighting for allergies may be high while a weighting for COVID-19 may be low. Accordingly, the analytic server can create tailored datasets to particular diseases to more accurately determine risk scores for individuals or their observer devices. The analytic server may do so based on the signals that individuals' observer devices transmit and detect from other observer devices or electronic devices in hypercluster.

In some embodiments, because the labels of hyperclusters may be associated with specific locations within a building, the analytic server may determine the locations within the building to which a person traveled during a time period based on observation data associated with the hyperclusters with which an observer device of the person was proximately associated during the time period. The analytic server may identify the proximately associated hyperclusters and the labels identifying the locations within the building of the proximately associated hyperclusters to determine where the person was within the building during the time period. As described above, because each location may be associated with a weight, the analytic server may be able to determine a risk score for the observer device based on the locations of the observer device during the time period.

At step 1314, the analytic server may transmit a signal comprising the risk score to a client device. The analytic server may transmit the signal responsive to determining the risk score for the observer device. The analytic server may transmit the signal to a client device that sent a query to the analytic server to determine the risk scores for various observer devices. The signal comprising the risk score may include a graphical user interface including the risk score and, in some cases, other information detailing how the risk score was determined. For example, the graphical user interface may include a list of the hyperclusters and their respective labels that the analytic server identified as the observer device having been proximately associated with within a time frame of the query. The graphical user interface may also include details on the weighting that was used to determine the risk score. The graphical user interface may include any information. In some embodiments, transmitting the risk score for the observer device may cause the client device to send an alert to the observer device indicating the risk score. In some embodiments, the client device may send the alert responsive to determining the risk score exceeds a threshold.

### VIII. Determining Risk Score Using Contextualized Data

Embodiments herein describe systems and methods for generating a risk score based on sets of contextualized data. It is difficult to determine proximal relationships between electronic device (e.g., wireless communications devices) in a world where their locations may not be known and/or are continuously changing. Determining device relationships can reveal groups of similar devices and describe properties of different classes of physical spaces (e.g., hotels, restaurants, etc.), in a similar fashion to systems such as recommendation and classification engines. Systems and methods may enable a server to determine and use data from such groups of physical devices to determine characteristics about individual devices and/or the users associated with the individual devices. For example, some systems may determine risk scores for individuals indicating the likelihood that the individuals have an infectious disease (and in some cases a degree of infections) such as COVID-19. However, it can be difficult to determine risk scores for such diseases while the environment around the individual is ever-changing. For example, systems that do not implement the systems and methods discussed herein may use static data that is independent of any environmental factors that may impact such risk scores. Such systems may take the data that they receive at face value when determining risk scores for observer devices for infectious diseases and not take into account contextual features of the data that may have a substantial impact on the risk score.

In an example embodiment, a system implementing the systems and methods described herein may use groupings of signals to identify different proximal groupings of signals with which a person was associated during a time period (e.g., a day, week, two weeks, etc.). The system may leverage data gathered from the proximal groupings of signals to generate risk scores for individuals indicating a likelihood that an individual is infected with a disease such as COVID-19. To do so, the system may determine weights for labels representing areas within the building. The system may determine the weights based on the context of the area such as the activities that are performed in the area, characteristics of the area (e.g., number of surfaces), and the number of people that enter the area during a time period such as a day. Each of the context factors may impact the effect that entering the area may have on a user's chance of contracting an infectious disease. When determining the risk score for an observer device based on data within a time period (e.g., two weeks), the system may generate a dataset that includes the weights for the areas (represented by a label of a set of signals in the areas) that the user entered during the time period. The system may include weights specific to each day to account for environmental factors that may kill off or add viruses of infectious diseases into the area. The system may generate the dataset to include data about each instance that the user was in each area and the weights associated with the areas. The system may use the dataset to determine a risk score for the observer device.

Accordingly, embodiments herein recite systems and methods for determining a risk score based on sets of contextualized data. Embodiments disclosed herein solve the aforementioned problems and other problems.

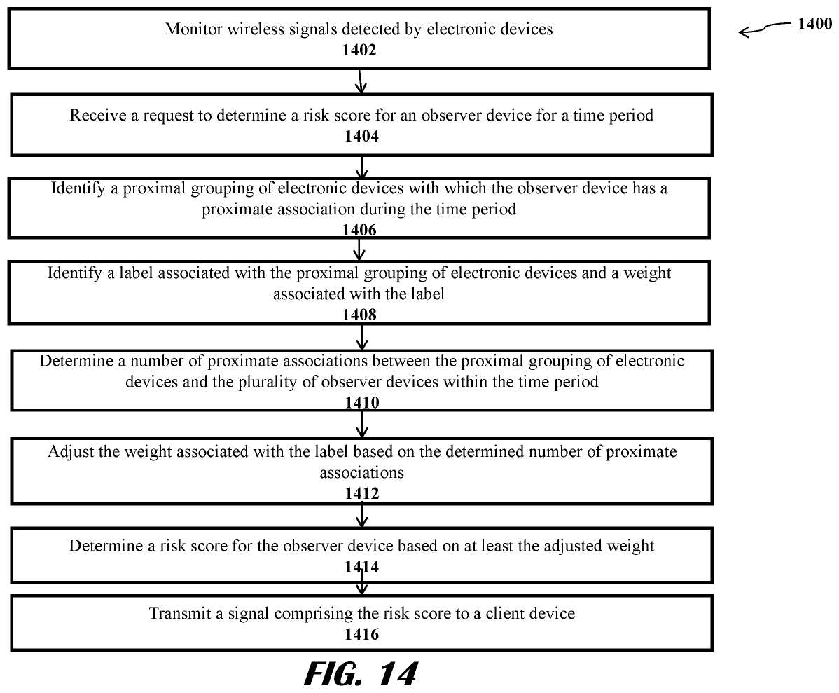

FIG. 14 shows a flow diagram 1400 of a method of generating a risk score based on sets of contextualized data, according to an illustrative embodiment. Other embodiments may comprise additional or alternative steps, or may omit some steps altogether. Although multiple computing systems and databases can implement one or more steps of the method, this description details, for brevity, an analytic server implementing the various steps of the method.

At step 1402, the analytic server may monitor wireless signals detected by electronic devices, which include observer devices. The analytic server may monitor the wireless signals using the techniques and methods as discussed herein.

At step 1404, the analytic server may receive a request to determine a risk score for an observer device for a time period. In some instances, the request may be a generic request to determine a risk score for each observer device that is associated with an entity such as a company. In some embodiments, the request is specific to the observer device and may comprise an identifier of the observer device. The identifier may be a BLE standard unique identifier. Alternatively, the identifier may be a MAC (media access control) address. In operation, a user may open a website in an Internet browser or a local application on an electronic client device configured to receive a request from the user. The analytic server may display a graphical user interface (GUI) for the user to input the request. For example, the user interface may include a text-based interface where the user can manually type requests and provide identifiers of target devices using a keyboard. In another example, the user interface may include an audio-based interface where the user can issue requests by verbally requesting a service.

At step 1406, the analytic server may identify a proximal grouping of electronic devices (e.g., a hypercluster) with which the observer device has a proximate association during the time period. The analytic server may do so based on observation data of the hypercluster. The hypercluster may be a set of signals or devices at a physical location within a building. For example, the hypercluster may be located in a kitchen or a conference room that includes a BLE beacon. The observer devices that enter a range to exchange wireless signals with the BLE beacon may enter a range of the hypercluster associated with the BLE beacon and the kitchen. The observation data may comprise wireless signals of the observer device and other wireless signals of the surrounding environment. The analytic server may determine the observer device was proximate to the hypercluster at a time point within the time period based on the observation data. The analytic server may store associations between signals from devices of the hypercluster and the hypercluster itself in the database.

Wireless signals that observer devices detect and transmit to the analytic server may include time points. A time point may be the time that an observer device detected a wireless signal from another device of a hypercluster. In some embodiments, the analytic server determines that the other wireless signals of the surrounding environment are associated with the observer device based on the signals having time points that are the same as or similar to (e.g., within a range or threshold) time points of the wireless signal that the observer device detects. For example, as discussed above, the analytic server may build hyperclusters based on the monitoring of the plurality of electronic devices. Based on observations from the plurality of observer devices, the analytic server may determine the signal context, such as the hypercluster, of the observer device at a given time point by determining the hypercluster of the observer devices that detect wireless signals from the observer device. For example, the analytic server may query a list of detected wireless signals from the observer devices and determine the corresponding hypercluster. The analytic server may determine the hypercluster with which the observer device was associated (e.g., the hypercluster to which the observer device was has a proximate association) based on the observer device detecting and transmitting signals between the same observer devices of the respective hypercluster. The data that the analytic server stores related to the hypercluster may also be associated with a time point indicating when the wireless signals associated with the data were detected.

The hypercluster may provide information on the signal environment of the observer device, and further provide information on the physical environment. Because a hypercluster may be a set of signals that have been observed together within a number of observations, a given hypercluster may represent a set of devices that remain in physical proximity over time. In other words, a hypercluster may correspond to a physical location. The analytic server may determine the physical location based on the hypercluster. For example, the analytic server may retrieve data from the database storing the hyperclusters and the corresponding locations.

In some embodiments, upon receiving the request to determine the risk score for the observer device, the analytic server may identify observation data from the database that is associated with time points within the time period. The analytic server may do so by filtering out data with time points outside of the time period. The analytic server may do so by removing such data from a list of observation data. The analytic server may compare the time points of observation data to the time period and remove observation data that is not within the time period. For example, the analytic server may receive a request to determine a risk score for an observer device over the immediately previous two weeks. The analytic server may identify the observation data from the immediately previous two weeks in a dataset and exclude any observation data that is not associated with time points within the two week period. Consequently, the analytic server may create a list of observation data that is associated with time points within the time period.

The analytic server may identify a hypercluster that is associated with observation data within the time period. The analytic server may identify any number of hyperclusters that are associated with observation data from within the time period. The analytic server may identify the hypercluster by identifying the hypercluster that is associated with observation data associated with the observer device (e.g., observation data indicating the observer device was proximate to the hypercluster) within the time period.

At step 1408, the analytic server may identify a label that is associated with the proximal grouping and a weight associated with the label. A label may be a flag or setting that is stored in the database. A label may be a string of characters. A label may be an identifier of a particular hypercluster. In some embodiments, a label may be an indicator of a type of a hypercluster. A type of a hypercluster may be associated with a location (e.g., a type of room such as a kitchen, a gym, a personal office, a hallway, etc.) of a hypercluster within a building or on a premises including a building and grounds around the building, types of activities (e.g., eating, exercising, working, singing, yelling, whispering, etc.) that are performed in a location of a hypercluster, the types of electronic devices of the hypercluster (e.g., mobile phones, headphones, televisions, laptops, LTE Beacons, Bluetooth Beacons, Wi-Fi routers, etc.), etc. In some embodiments, labels may be associated with a combination of the location of the hypercluster and the types of the devices that are associated with the hypercluster. For example, a label may be “gym—headphones.” Such a label may indicate that the hypercluster is associated with a location within a gym and that at least one of the devices associated with the hypercluster is a set of headphones. The labels may be associated with any permutation of devices and/or locations. Such labels may be associated with varying weights and/or other values that the analytic server may use to analyze observation data that it collects, as described herein. In some embodiments, the weights may be associated or matched with the respective hypercluster itself. The analytic server may store associations between the hyperclusters and such labels in a database.

In some embodiments, observer devices of hyperclusters may transmit labels to the analytic server with the detected wireless signals from devices within the hypercluster with which they are associated. Observer devices may include such labels in the header or body of data packets that include data of the detected wireless signals that the observer devices transmit to the analytic server. The observer devices may be configured (e.g., via an input) to transmit such labels to the analytic server. For example, a BLE beacon in a kitchen may be configured to transmit a signal with a label indicating that the BLE beacon is associated with a kitchen label along with any detected wireless signals that the BLE beacon transmits to the analytic server. The analytic server may receive such signals and store data from the wireless signal in the database along with an association between the kitchen label and the detected wireless signals. In another example, a BLE beacon may be configured to transmit a signal with a label indicating that the BLE beacon is in the hallway. The analytic server may store associations between the label and the wireless signals that the BLE beacon provides in the database so the analytic server may identify the labels and characteristics of the labels (e.g., weights) when analyzing the data of the wireless signals.

In some embodiments, an administrator may associate instances of hyperclusters stored in the database with labels. An administrator may do so by inputting labels into a user interface to label instances of hyperclusters within the database with labels indicating the respective types of the hyperclusters. For example, an administrator may, via a user interface displayed on a client device (e.g., a human resources device) being accessed by the administrator, view a list of hyperclusters that have been instantiated in the database. In some cases, the user interface may additionally display the electronic devices that are associated with the respective hyperclusters. In some embodiments, the analytic server may generate the user interface. For one or more of the hyperclusters, the administrator may select the hypercluster and/or input a label to associate with the particular hypercluster. The analytic server may receive the input and update the database by storing an association between the hypercluster and the label. For example, the analytic server may receive an input from a client device operated by an administrator indicating that a particular hypercluster is associated with the label “gym.” The analytic server may identify the gym label and the hypercluster associated with the gym label and store an association between the gym label and the hypercluster in the database. Any data that the analytic server receives that is associated with the gym hypercluster may be associated with the gym label within the database. Consequently, the analytic server can maintain the database of hyperclusters and labels that are associated with the hyperclusters to analyze observation data.

In some embodiments, the analytic server automatically determines a label for a hypercluster based on the devices that are associated with the respective hypercluster. The analytic server may store associations between labels and the observer devices in the database. The analytic server may use the stored associations between the labels and the observer devices to identify the labels to associate with the respective hyperclusters. To do so, the analytic server may identify the devices that are associated with a hypercluster and determine if any labels are associated with the devices. The analytic server may make such a determination by scanning the database to determine if it has any stored associations between the devices and labels. Responsive to identifying a stored association between an observer device of a hypercluster and a label (e.g., an identifier of the observer device and the label), the analytic server may retrieve the label from the database and determine the retrieved label is associated with the hypercluster. For example, the analytic server may generate a hypercluster that is associated with a BLE beacon in a kitchen, a first observer device, and a second observer device. The analytic server may scan the database to determine if any of the BLE beacon, the first observer device, or the second observer device are associated with a label. The analytic server may identify a label associated with the BLE Beacon with the string “kitchen” based on the scanning. Accordingly, the analytic server may identify “kitchen” as the label that is associated with the hypercluster that is associated with the BLE Beacon, the first observer device, and the second observer device. Advantageously, by automatically associating hyperclusters with labels based on labels associated with devices, the analytic server may determine labels for newly generated hyperclusters and generate data for the analytic server to use to determine risk scores without any user input.

The analytic server may store an association between the hypercluster and the label in a database. The first association may be an instance of a tag or setting that the analytic server stores in the database. The analytic server may determine the label that is associated with the hypercluster using any one of the methods described above. Once the analytic server has determined the label, the analytic server may store an association between the observer device and the first label in the database. Upon processing observation data associated with the first hypercluster, the analytic server may identify the label associated with the hypercluster from the stored association.

In some embodiments, the labels and/or hyperclusters are associated with weights. For instance, the first label may be associated with a first weight. Weights may be the magnitude of the impact the observer device being proximate to a hypercluster may have on a risk score that the analytic server determines for the observer device. As will be described below, the analytic server may use the weights of labels to determine a risk score for the observer device. Weights may range from 0-1, 1-10, 1-100, or any range. The weights may be associated with the respective location of the hypercluster, the actions that are performed in the location, the number of people that are generally around the location, etc. For example, the kitchen may be associated with a high weight because of the number of surfaces in the kitchen that could carry bacteria or viruses while a conference room may have a low-risk score because of low through-traffic of people.

In some embodiments, weights associated with labels and/or hyperclusters are input by an administrator at a client device. For example, via a user interface, an administrator may input a weight of 0.8 for the gym label and a weight of 0.2 for an outside garden. The analytic server may receive the weights and store associations between the weights and the labels and/or hyperclusters associated with the labels in the database.

At step 1410, the analytic server may determine a number of proximate associations between the proximal grouping of electronic devices and the plurality of observer devices within the time period. During the time period, one or more observer devices of the second hypercluster may transmit data to the analytic server indicating that the respective observer device is proximate to the second hypercluster. For example, during the course of a day, a number (e.g., 1, 10, 100, 500, etc.) of observer devices of a hypercluster may transmit signals to the analytic server indicating that they are proximate to the hypercluster. The analytic server may receive each of these signals and maintain and increment a counter associated with the respective hypercluster for each signal the analytic server receives from an observer device. A number of the counter may indicate the number of instances that observer devices were proximate to the hypercluster during the time period.

At step 1412, the analytic server may adjust the weight associated with the label based on the determined number of proximate associations. The analytic server may adjust the weight by increasing or decreasing the weight associated with the label. The analytic server may adjust the weight for the determination of the risk score in response to the request. The analytic server may determine whether to increase or decrease the weight for the label based on the counter associated with the label for the time period. For instance, responsive to the count being high, the analytic server may increase the weight for the label. However, responsive to the count being low, the analytic server may decrease the weight for the label. For example, the weight associated with the “kitchen” label may be 0.8 on a scale from 0 to 1. The analytic server may determine the count associated with the kitchen was low. Consequently, the analytic server may decrease the weight for the kitchen by 0.2 so the weight becomes 0.6. In another example, the weight associated with a conference room may be 0.3. The analytic server may determine the count associated with the conference room to be high. Consequently, the analytic server may increase the weight for the conference room by 0.3 so the weight becomes 0.6. The analytic server may adjust the weights associated with any labels.

When adjusting the weight for the determination of the risk score, the adjustment may be specific to calculations performed for the time period and/or request. For example, the analytic server may adjust the weight associated with the label to determine the risk score for an observer device and, upon determining the risk score, change the weight of the label back to original weighting. Accordingly, when the analytic server next determines risk scores for an observer device, the analytic server can use the original weighting for the label and adjust it specific to the factors that are specific (e.g., time period, number devices that become proximate to the hypercluster associated with the label, etc.) to the request. For example, after receiving the risk score for the observer device in response to its request, the client device may transmit a second request to the analytic server to determine the risk score for another observer device over a later second time period. In response to receiving the request, the analytic server may adjust the weighting of the labels of the hyperclusters with which the other observer device is associated based on the weighting of the label and irrespective of any adjustments the analytic server made to respond to the previous request. In some embodiments, the analytic server generate a new weight for the label when determining the risk score, the new weight may match the weight associated with the label that is stored in the database. The analytic server may adjust the new weight and associate the adjusted new weight with data that is generated from the hypercluster within the time period.

In some embodiments, the analytic server may adjust the weight for the label based on a comparison of the number of the counter to a baseline. The baseline may be associated with the label and may indicate an expected or average number of instances in which observer devices become proximate to the hypercluster associated with the label. For example, a baseline for a kitchen label may be 500 instances of proximation with observer devices while a baseline for a conference room label may be 50 instances of proximation with observer device because more people generally visit the kitchen in an office building than visit the conference room. The analytic server may compare the counter to the baseline for the label associated with the hypercluster and adjust the weight associated with the label based on the difference between the counter and the baseline. For instance, responsive to determining the counter is lower than the baseline for a label, the analytic server may decrease the weight associated with the label. Responsive to determining the counter is higher than the baseline for the label, the analytic server may increase the weight associated with the label. The analytic server may adjust the weight proportional to the magnitude of the difference between the counter and the baseline. For example, if the counter is much larger than the baseline, the analytic server may increase the weight by a relatively large amount. If the counter is much smaller than the baseline, the analytic server may decrease the weight by a large amount.

In some embodiments, the analytic server adjusts the weight for the label for sub-time periods within the time period of the request. The sub-time periods may be every 30 minutes, hour, day, etc., as configured by an administrator, within the time period. For example, the request for the risk score may be for a two week time period. The analytic server may individually adjust the weight for the label of the hypercluster on a per-day basis so the label may be associated with a different weight each day of the two week period. The analytic server may determine the number of observer devices that became proximate to the hypercluster of the label each day and adjust the weight associated with the label for each day accordingly, as discussed above. For each adjustment, the analytic server may adjust the original weight for the label to determine the weight of the sub-time period. For example, if a kitchen label has a weight of 0.8, the analytic server may increase the weight by 0.1 to 0.9 one day and decrease the weight by 0.2 to 0.6 the next day. Consequently, when determining the risk score for an infectious disease like COVID-19, the analytic server may take into account that the virus dies in the air over time and that instances that observer devices entered a hypercluster one day may not impact the chances the weighting of for the hypercluster the next day because the virus may have died in the air during the respective day (or other sub-time period). The analytic server may store an association between the label, the respective day, and the adjusted weight in the database to use when determining the risk score for the observer device.

Advantageously, by accounting for the number of devices and/or the number of instances that such devices have a proximate association with the analytic server during a time period for which the analytic server is determining a risk score, the analytic server may more accurately take into account factors that are specific to a particular disease. For example, for COVID-19, a person may be more likely to contract the disease if the person is in an area of a building that has a lot of foot traffic. Because COVID-19 can linger in the air, the more people that enter the area of the building over the course of a time period (e.g., a day), the higher the odds that a person will contract COVID-19 when entering the area, even if there are not any people there at the time the person is in the area. By adjusting the weights for labels that may represent such areas based on the number of instances that devices are proximate to the hypercluster associated with such areas, the analytic server may dynamically determine weights specific to the hypercluster and/or label. The analytic server may use the dynamically determined weights to determine the risk score for an observer device that the analytic server identified as being associated with the same hypercluster, improving the accuracy of the determination compared with systems with static weights that do not take changing environmental factors into account when determining risk scores.

At step 1414, the analytic server may determine a risk score for the observer device based on at least the adjusted weight. The analytic server may determine risk scores for the observer device based on the adjusted weight by applying one or more algorithms to a dataset that includes data identifying the instances the observer device was proximately associated with various hyperclusters, the weights and/or labels associated with such hyperclusters, and/or a length of time that each proximate association between the respective observer device and the hypercluster occurred (e.g., a length of time the observer device was proximately associated with the respective hypercluster). The analytic server may identify the weight for the label of each hypercluster for which the observer device has a proximate association during the time period and for each sub-time period within the requested time period when determining the risk score for the observer device. For example, the analytic server may determine a risk score for an observer device based on data within a time period. The analytic server may do so by identifying the weights for the labels for each sub-time period within the time period and multiplying the weights of data associated with time points within the sub-time periods and the observer device by the number of instances that the observer device was proximately associated with each respective hypercluster to obtain weighted proximate associations. The analytic server may aggregate the weighted proximate associations to determine the risk score. In some embodiments, continuing with this example, the analytic server may determine the length of each proximate association and further multiply the weighted proximate associations by their respective lengths before aggregating the weighted proximate associations to determine the risk score for the observer device. In another example, the analytic server may only use proximate associations that are associated with weights that exceed a threshold when determining the risk score. The analytic server may compare the weights of proximate associations to a threshold and only aggregate weighted proximate associations that are associated with weights that exceed the threshold. Thus, to determine risk scores for diseases such as COVID-19, the analytic server may not take into account noise that does not increase a person chances of getting the disease but when aggregated together may have a substantial impact on determining the risk score for the person's observer device, which could cause the risk score to be inaccurate. In some embodiments, the analytic server may further take into RSSI data to determine the risk score. For example, signals associated with higher RSSI values may be associated with higher weights than signals associated with lower RSSI values. Any algorithm may be used to determine a risk score for a person or observer device.

At step 1416, the analytic server may transmit a signal comprising the risk score to a client device. The analytic server may transmit the signal responsive to determining the risk score for the observer device. The analytic server may transmit the signal to the client device that sent the request to determine the risk score for the observer device. The signal comprising the risk score may include a graphical user interface including the risk score and, in some cases, other information detailing how the risk score was determined. For example, the graphical user interface may include a list of the hyperclusters and their respective labels that the analytic server identified as the observer device having been proximately associated with within a time frame of the query. The graphical user interface may also include details on the weighting and any adjustments to the weighting that were used to determine the risk score. The graphical user interface may include any information. In some embodiments, transmitting the risk score for the observer device may cause the client device to send an alert to the observer device indicating the risk score. In some embodiments, the client device may send the alert responsive to determining the risk score exceeds a threshold.

### IX. Generating a Risk Score Based on Predictive Data

Embodiments herein describe systems and methods for generating a risk score based on predictive data. Observation data that is collected from observer devices may be used to determine risk scores for infectious diseases for users. A server may aggregate the observation data associated with an observer device for a series of days to determine a risk score for the observer device (as a proxy for a user). The server may use risk factors associated with particular locations that the user visits during the time period to determine such a risk score. However, in some instances, a user may not be in the same location as observation data associated with their device indicates. The user may have left their phone at home, dropped it outside, left it in their office, etc. In some cases, the observer device may not be associated with any observation data for a time period at all. Such may be the case as a result of their observer device turning off, running out of battery, or being located outside of a geofenced area that enables it to transmit signals. Because the environmental factors of the physical location of the user may be important for determining a risk score, there is a need to determine which location the user was in at a time point irrespective of observation data associated with the user's observer device.

Advantageously, by implementing the systems and methods described herein, a server may automatically determine the location of a user at a time point irrespective of the wireless signals that are associated with the user's observer device at the time point. The server may use historical observation data to determine a pattern of locations of the observer device for a given day or time period to determine expected locations for the observer device throughout the day or time period. When determining a risk score for the observer device, the server may compare the expected locations with observed locations of the observer device. Responsive to the server identifying a conflict, the server may determine the user associated with the observer device was likely at the expected location and use observation data associated with the expected location when determining the risk score for the observer device, in some cases instead of observation data of the observed location. The server may use a weight associated with the expected location on the observation data of the time period to determine the risk score for the observer device. Consequently, the server may determine a more accurate risk score for observer devices based on location patterns the server has identified for the observer device.

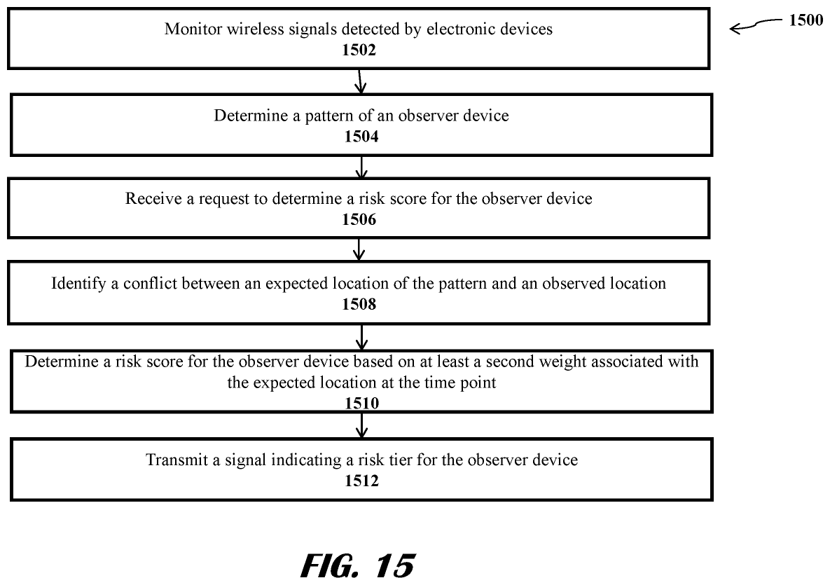

FIG. 15 shows a flow diagram 1500 of a method for determining a risk score based on predictive data, according to an illustrative embodiment. Other embodiments may comprise additional or alternative steps, or may omit some steps altogether. Although multiple computing systems and databases can implement one or more steps of the method, this description details, for brevity, an analytic server implementing the various steps of the method.

At step 1502, the analytic server may monitor wireless signals detected by a plurality of electronic devices (including observer devices). The analytic server may monitor the wireless signals using the techniques and methods as discussed herein.

At step 1504, the analytic server may determine a pattern of an observer device. The analytic server may determine a pattern for an observer device based on observation data that the analytic server collects from the plurality of observer devices. The observation data may comprise wireless signals of the observer device and other wireless signals of the surrounding environment. The analytic server may determine the observer device was proximate to the groups of signals (e.g., hyperclusters) based on the observation data.

The hypercluster may provide information on the signal environment of the observer device, and further provide information on the physical environment. Because a hypercluster may be a set of signals that have been observed together within a number of observations, a given hypercluster may represent a set of devices that remain in physical proximity over time. In other words, a hypercluster may correspond to a physical location. The analytic server may determine the physical location of observer devices based on the hypercluster. For example, the analytic server may retrieve data from the database storing the hyperclusters and the corresponding locations.

In some embodiments, the analytic server may determine the location of the observer device based on labels associated with the hyperclusters with which the observer device is proximate. As discussed herein, labels may correspond to locations within a building. Such labels may be stored on an observer device (e.g., a BLE beacon or a Wi-Fi router) and transmitted to the analytic server with detected signals from observer devices. The analytic server may identify the labels of the signals and associate the signals with the respective hypercluster in the database. Consequently, the analytic server may store labeled data associated with the physical locations of the observer device around a building that was generated throughout the day or within another time period.

In some embodiments, the analytic server may determine patterns of the observer devices interactions with one or more of the hyperclusters. For example, the analytic server may determine that the observer device detects wireless signals in one location in the morning, another location during business hours, and yet another location again in the evening and overnight. Based on this determination, the analytic server may indicate that the observer device adheres to or approximates movements throughout a time period (e.g., a day). Each of the locations may be within an office building or within a geofenced area of the office building, as discussed herein.

In some embodiments, to determine the pattern, the analytic server may maintain counters indicating the time points or time ranges for which the observer device was at different locations. Each location may be associated with a series of counters associated with different time points or time ranges throughout a time period. For example, the analytic server may maintain counters that are associated with hour time ranges throughout a day (e.g., a 24-hour period) of a time period of one year. The analytic server may maintain counters for any time ranges and/or time periods. For instance, the analytic server may maintain counters for the kitchen of a building for 1:00, 2:00, 3:00, etc. Upon determining or detecting that the observer device was in the kitchen at 1:00 and 3:00, the analytic server may increment the counters associated with 1:00 and 3:00. Responsive to the analytic server detecting that the observer device was in the kitchen at 1:00 and 3:00 the next day, the analytic server may increment the same counters. Consequently, the analytic server may keep track of the number of instances the observer device is at one or more locations over time.

In some embodiments, the analytic server may compare each of the counters to a threshold to determine whether a particular location is an expected location for a time point. The threshold may be a value stored in a database of the analytic server or a database from which the analytic server may retrieve data. Responsive to a counter for a time point and location exceeding the threshold, the analytic server may determine the location associated with the counter is an expected location at the time point. The analytic server may generate or determine a series of expected locations for an observer device throughout a day to determine an expected pattern of movement for the observer device throughout the day.

At step 1506, the analytic server may receive a request to determine a risk score for the observer device. The analytic server may receive a request to determine a risk score for an observer device for a time period. The time period may be one week, two week, a month, a year, etc. In some instances, the request may be a generic request to determine a risk score for each observer device that is associated with an entity such as a company. In some embodiments, the request is specific to the observer device and may comprise an identifier of the observer device. The identifier may be a BLE standard unique identifier. Alternatively, the identifier may be a MAC (media access control) address. In operation, a user may open a website in an Internet browser or a local application on an electronic client device configured to receive a request from the user. The analytic server may display a graphical user interface (GUI) for the user to input the request. For example, the user interface may include a text-based interface where the user can manually type requests and provide identifiers of missing devices using a keyboard. In another example, the user interface may include an audio-based interface where the user can issue requests by verbally requesting a service.

At step 1508, the analytic server may identify a conflict between an expected location of the pattern and an observed location. The conflict may be an indication that the observed location of the observer device at a time point was different than an expected location of the pattern at the same time point. The analytic server may make such a determination by identifying the location of the observer device throughout the time period of the request and compare each location to expected locations of the pattern of expected locations. Responsive to identifying an instance in which the observed location of the observer device is different than the expected location for a given time point, the analytic server may identify a conflict.

For example, for a time point the analytic server may identify the observer device as being in an office. The analytic device may identify the expected location for the observer device at the time point as the kitchen. The analytic server may compare the two locations and determine there is a conflict for the time point.

In another example, the analytic server may observe a period in which there is not observation data for the observer device. Such a period may occur when the observer device runs out of battery, is turned off, or is outside of a range of a geofencing associated with a building (e.g., outside of an administrator input polygon) and does not transmit any detected signals to the analytic server to be stored. Because the analytic server may not identify any observation data from this period when determining the risk score for the observer device, the analytic server may determine that the time points conflict with the observed pattern for each time point within the period.

At step 1510, responsive to identifying the conflict, the analytic server may determine a risk score for the observer device based on at least a weight associated with the expected location at the time point. The analytic server may identify the weight associated with the expected location from the database as the weight that is associated with the hypercluster of the expected location. The analytic server may apply the identified weight to observation data from the expected location at the respective time point and use weights and observation data from within the time period to determine the risk score, as discussed herein.

As described herein, weights are values that may be stored in a database of the analytic server or a database from which the analytic server can retrieve the value. The analytic server may use the weight in combination with risk factors for a particular infectious disease to determine a risk score for a user via their observer device. Examples of risk factors may be a length of time that the observer device was at a location (e.g., was proximate to a hypercluster), RSSI values for the wireless signals of the respective observation data, and/or identifications of infected observer devices that were at the same location as the observer device at the same time or at a different time. The analytic server may multiply the weights by any of the risk factors when determining the risk score for the observer device. The risk score may indicate a likelihood that the user has a particular infectious disease.

In some embodiments, the analytic server may determine the risk score for the observer device using the weight associated with the expected location by identifying observation data that is associated with the expected location at the time point of the conflict. Despite the observation data not being associated with the observed location of the observer device, the analytic server may determine that the user associated with the observer device was likely in the expected location at the time point. Consequently, the analytic server may determine the user was susceptible to the same infection factors as if the observer device were in the expected location at the time point. The analytic server may use a combination of the weight of the expected location and the observation data at the time point to determine the risk score for the observer device. The analytic server may similarly use observation data from other conflicts within the time period when determining the risk score. The analytic server may do so in combination with other observation data and weights of the requested time period.

In some embodiments, the analytic server may determine the risk score for an observer device by excluding observation data that is associated with the observed location at the time point of the conflict. The analytic server may exclude the observation data associated with the observed location when determining the risk score by filtering out such observation data when generating a dataset to determine the risk score for the observer device. The analytic server may exclude any observation data that is associated with a conflict between the expected location and the observed location of the observer device. The analytic server may apply weights that are associated with the observation data of the datasets excluding the observation data at the time point of the conflict to determine the risk score.

In some embodiments, the analytic server may determine to exclude observation data associated with an observed location at the conflicting time and/or determine to use observation data associated with the expected location instead responsive to determining that a probability that the user associated with the observer device was not at the observed location exceeds a threshold. To do so, the analytic server may analyze a historical pattern of the observer device and determine the probability of the observer device being in the current location based on the historical pattern. The analytic server may analyze the counters that it maintains to determine the number of instances that the analytic server has observed the observer device at the expected location, in some cases within a most recent time period as input by an administrator. The analytic server may determine a probability that the user associated with the observer device was at the expected location at the time based on the determined number of instances. The analytic server may compare the probability to a threshold and, responsive to the probability exceeding the threshold, the analytic server may use observation data and/or the weight associated with the expected location at the time point to determine the risk score for the observer device. Responsive to the probability not exceeding the threshold, however, the analytic server may use the data associated with the observed location, if it is available, to determine the risk score for the observer device.

At step 1512, the analytic server may transmit a signal indicating a risk tier for the observer device. The analytic server may transmit the signal to a client device that sent the request to the analytic server. A risk tier may be a risk level indicating or corresponding to a risk of exposure of a user to disease. Examples of risk tiers include a high risk, a medium risk, a low risk, etc. The high risk may correspond to, for example, a centers for disease control and prevention (CDC) guideline. The server may generate the risk level data indicating the determined risk level of the target device, and transmit the risk level data to the requesting device (e.g., client device).

To determine the risk tier for the observer device, the analytic server may compare the determined risk to risk thresholds associated with each tier. The risk thresholds may be values that are stored in a database of the analytic server or a database from which the analytic server may retrieve data. Each risk threshold may be associated with a risk tier (e.g., low, medium, high, etc.). Responsive to determining that the risk score satisfies a particular risk threshold, the analytic server may determine the observer device is associated with the risk tier associated with the threshold. Upon determining the risk tier for the observer device, the analytic server may transmit a signal to the observer device indicating the determined risk tier.

### X. Additional Embodiments

In some embodiments, the analytic server can build location maps indicating the location of one or more observer devices at various points in time. For example, an observer device can capture a wireless signal emitted from one or more neighboring wireless devices, where the wireless signal includes an observer ID associated with the wireless device and geolocation data indicating the location (e.g., latitude, longitude) of the wireless device when it transmitted the wireless signal. The observer device can include the geolocation data that it captures from each wireless device in the observation data that it generates and sends to the analytic server. In response to receiving a request (e.g., a tracing data request) from the HR server, the analytic server can generate tracing data that includes a plurality of observation identifiers, a plurality of risk scores, a plurality of geolocation data, and/or a plurality of identifiers to hyperclusters. That is, each observation identifier of the plurality of observation identifiers may be mapped (e.g., linked) to a respective risk score, a respective set of geolocation data, and an identifier to the hypercluster that contains the respective observation device. The analytic server can send the tracing data to the HR server. In response to receiving the tracing data, the HR server can use an API (or plug-in application) to read the location data in order to display the locations of the observer devices on a map (e.g., satellite view, topography view, street view, etc.). Accordingly, an HR representative can determine whether the location/movement of the observer devices, as displayed on the map, should warrant sending the employee associated with the observer device a notification to self-quarantine.

In some embodiments, an administrator may tailor the notifications that it transmits to devices based on hyperclustering contacts, device contacts, and/or “tiered” business rules. For example, in response to receiving the tracing data from the analytic server, the HR server may determine whether to send a notification to an employee associated with an observer identifier that is included in the tracing data based on whether the employee is (or once was) within a hypercluster having a particular ranking. For example, if the employee identified in the tracing data is (or once was) within a hypercluster having a high risk rating, then the HR server can send a notification (alert) to the employee. As another example, if the employee identified in the tracing data is (or once was) within a hypercluster having a low risk rating, then the HR server can determine to not send a notification to the employee. In some embodiments, an observer device may share its device contacts with the HR server, by including the device contacts in the reporting data that it sends to the HR server. If the HR server receives tracing data that includes an identifier to the observer device, then the HR server can send notification to some or all employees that are listed in the device contacts for that observer device. In some embodiments, the HR server can determine whether to send a notification to an employee based on the tiered business rules. The tiered business rules may include, employee title, employee office location, employee working hours, employee age, etc.

For each of the retrieved contact information, the HR server may generate a message (shown in FIG. 6 as “alert data”) and transmits the message to the employee associated with the contact information to notify the employee to self-quarantine

In some embodiments, the analytic server receives, from a client device, a request to back-trace an observer device. The request may include an identifier of the observer device and a time period for the analytic server to conduct the trace. The request may cause the analytic server to analyze observation data associated with the observer device that was generated during the time period. The analytic server may analyze the observation data and determine the hyperclusters that the observer device came into contact with within the time period. The analytic server may identify the locations associated with such hyperclusters based on their labels. For example, the analytic server may perform a back-trace for an observer device to determine the hyperclusters with which the observer device was associated within the previous two weeks. The analytic server may identify each hypercluster from observation data that was generated within the two weeks. The analytic server may identify the labels associated with the identified hypercluster to identify the locations and/or other context of the hyperclusters. Furthermore, while conducting the back-tracing, the analytic server may identify the observer devices with which the observer device came into contact within the two week period. In some cases, the analytic server may determine the locations that the observer devices came into contact based on labels of hyperclusters that the observer devices were near when they came into contact. The analytic server may determine a risk score for the observer device based on the identified hyperclusters and observer devices.

In some embodiments, the analytic server uses machine learning or artificial intelligence algorithmic classifiers to determine associations based on the back-tracing described above. The classifiers may include neural networks, random forest, support vector machines, deduction models, or any other type of classifier. The analytic server may use such algorithmic classifiers to determine risk tiers or notifications or alerts. For example, after conducting or performing a back-trace for an observer device, the analytic server may input observation data that was generated within the two week time period into the classifier. The analytic server may also input the labels identifying the hyperclusters and/or other observer devices that the observer device came into contact with and the time points associated with the hyperclusters. The weights and/or parameters of the classifier may operate on the data to automatically determine risk tiers (e.g., high, medium, or low) or notifications or alerts (e.g., alerts identifying the risk tiers or indicating that the observer device is close to a particular risk tier). The classifiers may output these risk tiers or notifications or alerts. The analytic server may identify the output. In some cases, the analytic server may transmit the output to the client device.

In some embodiments, the server includes or implements a machine learning artificial reality algorithm that infers user behaviors of enabling or disabling tracing and/or privacy settings, and automatically enables or disables the tracing and/or privacy settings. For example, a user operating an observer device may manually enable or disable tracing or privacy settings at particular locations (e.g., restroom) or during a particular time (e.g., between 12:00 PM and 1:00 PM). The machine learning artificial reality algorithm may monitor the history of enabling and disabling the tracing or privacy settings, and infer or learn the pattern of enabling and disabling the tracing or privacy settings. Then, the machine learning artificial reality algorithm may automatically apply the tracing or privacy settings according to the inferred or learned pattern.

In some embodiments, the server receives a unique identification of a device (e.g., observer device) operated by a user that may be exposed to disease, and automatically determines or identifies one or more devices (e.g., observe devices) associated with the same hypercluster as the device. In one approach, the server receives a request, from the client device, to identify one or more devices that coexisted with or detected by the device. The server may automatically backtrack to identify one or more devices coexisted with the device in the same or nearby geographic area or shared the trajectory of the device. The server may identify one or more hyperclusters that coexisted with the device in the same or nearby geographic area or shared the trajectory of the server, and detect one or more devices in the identified one or more hyperclusters. The server may generate risk level data indicating risk levels of the identified one or more devices. The server may also provide a notification (e.g., push notification) to the identified one or more devices (or observer devices in the identified hyperclusters) to alert the risk levels. Advantageously, one or more devices operated by a user with a high likelihood of exposure to certain disease can be automatically identified, and an alarm or a warning message can be provided to users operating the identified one or more devices in a prompt and efficient manner.

### XI. Registered Observation and Tracer Devices

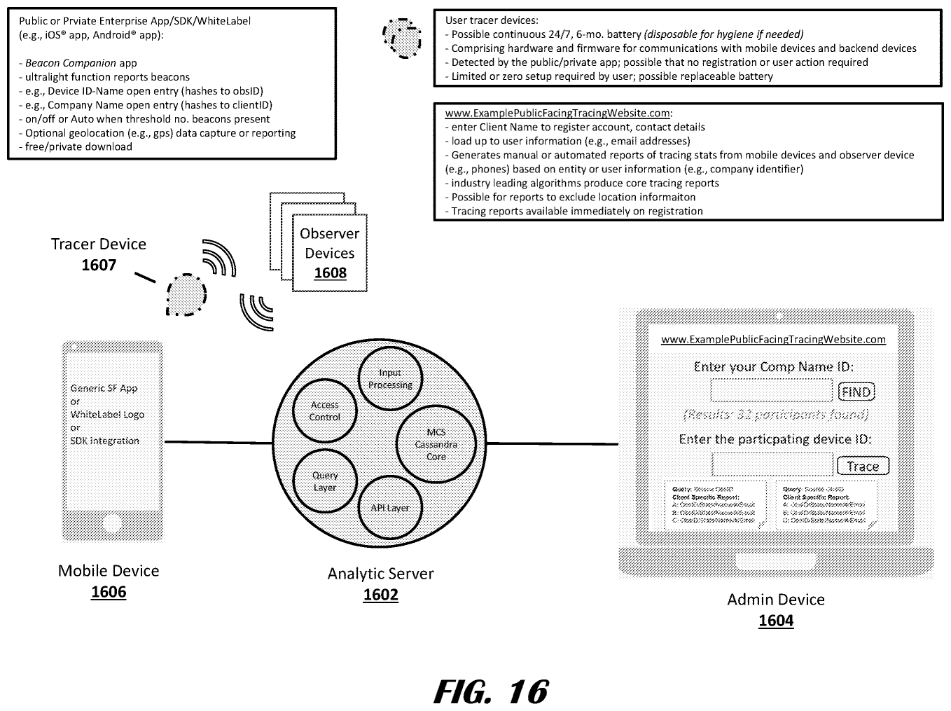

FIG. 16 shows components of a system 1600 according an embodiment. The illustrative system 1600 includes analytic servers 1602, admin devices 1604, mobile devices 1606, tracer devices 1607, and observer devices 1608. Although only a limited number of devices are shown in FIG. 16, it should be appreciated that embodiments can have any number of devices.

Observer devices 1608 are electronic devices that capture and communicate observation data with other components of the system 1600. Observer devices 1608 may be any electronic device having a processor, non-transitory machine-readable storage medium, and capable of performing the various processes and tasks described herein. Non-limiting examples of an observer device 1608 may include smartphones, laptops, tablets, beacon devices, and the like. The observer devices 1608 may emit signals containing observation data available for capture by other observer devices 1608, receive signals containing observation data emitted by observer devices 1608, or both.

In some embodiments, the observer devices 1608 can include mobile devices 1606 (e.g., smartphones, laptops, tablets) paired with tracer devices 1607 and execute a mobile application configured to communicate with analytic components of the system 1600. In such embodiments, the tracer devices 1607 emit the observation data on behalf the paired mobile devices 1606; the observer devices 1608 receive the wireless signals emitted by the tracer device 1607 containing various types of data about the mobile device 1606 paired with the tracer device 1607. Likewise, the mobile device 1606 receives the signals emitted by other observer devices 1608, which may be another tracer device 1607 or any other observer device 1608, and transmits the observation data to an analytic server 1602 or other device for performing various analytics. In the illustrative system 1600 of FIG. 16, the mobile device 1606 captures incoming observation data emitted in wireless signals from the observer devices 1608, but the mobile device 1606 does not transmit observation data. Rather, the tracer device 1607 paired with the mobile device 1606 emits the observation data about the mobile device 1606. As mentioned, other embodiments may vary how much operational functions are performed or shared by the mobile device 1606 and the paired tracer device 1607. In some implementations, the pairings between particular mobile devices 1606 and particular tracer devices 1607 may be predefined and stored into a database. Additionally or alternatively, the pairings may be dynamically determined by the analytic server 1602, such that mobile devices 1606 might not be permanently paired with tracer devices 1607.

The mobile device 1606 communicates observation data between observer devices 1608 of the system 1600 and reports observation data to analytic components (e.g., analytic server 1602) via one or more networks. The mobile device 1606 may emit observation data available for capture by other observation devices 1608, receive observation data emitted by observer devices 1608, or both. As mentioned, in some embodiments the mobile device 1606 is logically paired or otherwise associated with a particular tracer device 1607. In such embodiments, the mobile application or the operating system of the mobile device 1606 is instructed or otherwise configured to send observation information to the tracer device 1607, which then emits the wireless signals including the observation information about the mobile device 1607.

The mobile device 1606 may download and install the mobile application associated with the system 1600 from a public or private datastore. The mobile application instructs the mobile device 1606 on operations for capturing the observation data via the wireless signals and for reporting information to the analytic server 1602 via the one or more networks, such as the captured observation data and any configuration data. The configuration data reported with the observation data may include information the analytic server 1602 can analyze in order to, for example, determine which of the tracer devices 1607 is paired with which of the mobile devices 1606. Non-limiting examples of identification data for tracer devices 1607 and mobile devices 1606 may include a universal unique identifiers (UUIDs) within the system 1600 that is assigned or generated by the mobile application or the analytic server 1602, MAC addresses, and IP addresses, among others. In some cases, the identification data may include an identification for the mobile application in addition, or as an alternative, to the identification data for the mobile device 1607.

The analytic server 1602 executes various operations, such as tracing functions and updating a database, using the observation data and/or other types of data received from the various observer devices 1608 via one or more networks. In some embodiments, the analytic server 1602 can automatically determine whether the particular mobile device 1606 is associated with a particular tracer device 1607 using certain types of observation data received from the various observer devices 1607 and the mobile device 1606, such as timestamps, signal strengths, identifiers of the tracer devices 1607, identifiers of the observer devices 1608, and the like. The analytic server 1602 may, for example, compare the values of the observer data against various predetermined threshold values.

An admin device 1604 is a computing device that an administrator of the system 1600 uses to enter or update certain types of data and instruct the analytic server 1602 to perform the tracing functions. The admin device 1604 may access the analytic server 1602 through a graphical user interface (GUI), such as a web browser or native application that is locally installed on the admin device 1604. The administrator may complete an electronic form that include interface prompts allowing the administrator to input various types of registration data associated with the devices of the system 1600. The admin device 1604 may also be used by a user to send instructions to the analytic server 1602 to perform various tracing operations. Such operations are described in detail elsewhere herein, and are not further detailed here.

The registration data entered using the admin device 1604 may include, for example, data about an entity being serviced by the system 1600 (e.g., company name, company ID), the tracer devices 1607 (e.g., UUID, MAC address), the users (e.g., email address, user ID, username), the observer devices 1608 and mobile devices 1606 (e.g., UUID, MAC address), and the installed instances of the mobile application (e.g., UUID of the instance), among other types of data. The registration data is stored into a database, or other non-transitory storage medium of the system 1600, and may be accessed by the analytic server 1602 when executing certain processes. The registration data may indicate associations between users and the mobile devices 1606 or other observer devices 1608. For example, users may be associated with mobile devices 1606 or other observer devices 1608, which may be reflected in data records containing registration data. As mentioned, in some implementations, the database may store information indicating predetermined relationships between mobile devices 1606 and tracer devices 1607; and in some implementations, the relationships may be dynamically identified by the analytic server 1602, such that the relationships between mobile devices 1606 and tracer devices 1607 are not static.

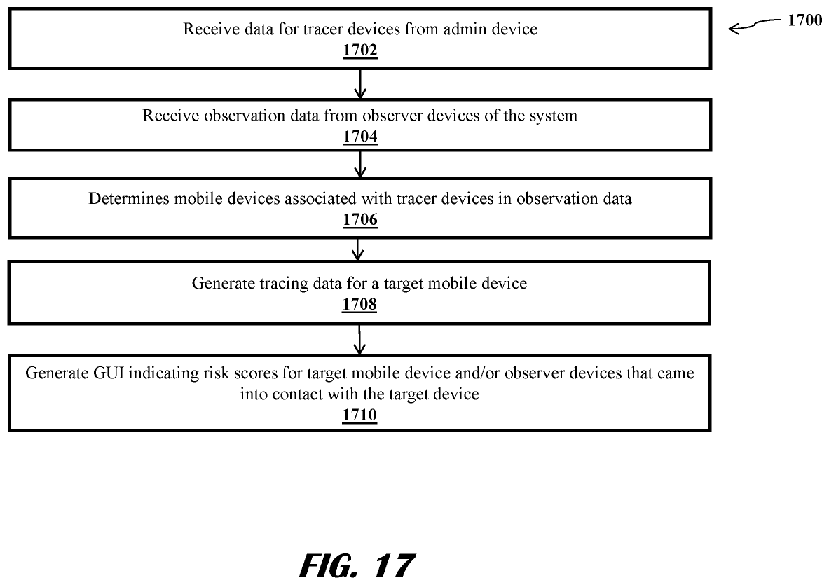

FIG. 17 shows execution steps of an illustrative method 1700 for contact tracing using a system with tracer devices, such as the illustrative system 1600 of FIG. 16.

In a first step 1702, an analytic server or other component of the system receives registration data from an admin device. The registration data indicating, for example, a company or entity identifier, tracer device identifiers, user identifiers, and/or mobile devices of the system. In some cases, the mobile device may transmit information from a mobile application associated with the system that is installed on the mobile device, such as universe unique identifier (UUID) for the instance of the mobile application.

In some embodiments, the system may be configured to require minimal registration information about users and/or mobile devices, such that the system receives data about the user and/or mobile devices during operation, without requiring a prerequisite registration process for the user or mobile device. In such embodiments, the registration data may be limited to, for example, information about the tracer device. It should be appreciated that the amount and type of information loaded into the system may vary between embodiments without departing from the scope of this disclosure.

In a next step 1704, the analytic server receives observation data from observer devices. The observer devices may include mobile devices (e.g., smartphones), among other types of wireless communication devices (e.g., beacons). Observation data received from a mobile device contains indicators of other devices or hyperclusters that the mobile device interacting with or captured data from through the course of operations. The mobile device reports the observation data captured during operation to the analytic server, by transmitting the captured observation data to the analytic server via one or more data networks. The observation data may further include indicators of tracer devices, such as a tracer identifier (tracer ID), from which the mobile device received observation data, where the tracer devices are associated with mobile devices of the system.

In step 1706, the analytic server identifies which mobile devices are associated with tracer devices included in the observation data. In some embodiments, the mobile devices are associated with tracer devices according to predetermined data entries. In such embodiments, database records, stored in a database accessible to the analytic server, may indicate that a mobile device is associated with a tracer device.

In some embodiments, the analytic server automatically determines that a tracer device is associated with a mobile device based upon one or more association rules, such as timing thresholds or observation thresholds, among other types of data. For example, the analytic server may receive updates from a mobile device identifying the tracer device paired with the particular mobile device. The analytic device may automatically determine that a particular mobile device is associated with a particular tracer device when, for example, the analytic server receives a number of reports indicating the tracer device is paired with the mobile device satisfies a threshold volume or timestamps of reports indicate the tracer device is paired with the mobile device satisfies a threshold time period.

In step 1708, the analytic server generates tracing data for a target mobile device. The tracing data indicates, for example, the various devices that the target mobile device interacted with over a given period of time. The tracing data for the target mobile device may be generated at a predetermined interval or time, or in response to a query received from the admin device. The analytic server identifies the target mobile device

In some embodiments, such as the illustrative method 1700, the tracing data may include one or more risk scores. In some implementations, the analytic server may generate a risk score for the target mobile device; and in some implementations, the analytic server may generate risk scores for each of the observer devices of the system that interacted with the target mobile device.

In step 1710, the analytic server generates a GUI indicating risk scores for target mobile device and/or observer devices that came into contact with the target device. The GUI may be generated and presented to the admin device that submitted the instructions to the analytic server.

The foregoing method descriptions and the process flow diagrams are provided merely as illustrative examples and are not intended to require or imply that the operations of the various embodiments must be performed in the order presented. The operations in the foregoing embodiments may be performed in any order. Words such as “then,” “next,” etc. are not intended to limit the order of the operations; these words are simply used to guide the reader through the description of the methods. Although process flow diagrams may describe the operations as a sequential process, many of the operations can be performed in parallel or concurrently. In addition, the order of the operations may be re-arranged. A process may correspond to a method, a function, a procedure, a subroutine, a subprogram, and the like. When a process corresponds to a function, the process termination may correspond to a return of the function to a calling function or a main function.

The various illustrative logical blocks, modules, circuits, and algorithm operations described in connection with the embodiments disclosed herein may be implemented as electronic hardware, computer software, or combinations of both. To clearly illustrate this interchangeability of hardware and software, various illustrative components, blocks, modules, circuits, and operations have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of this disclosure or the claims.

Embodiments implemented in computer software may be implemented in software, firmware, middleware, microcode, hardware description languages, or any combination thereof. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

The actual software code or specialized control hardware used to implement these systems and methods is not limiting of the claimed features or this disclosure. Thus, the operation and behavior of the systems and methods were described without reference to the specific software code being understood that software and control hardware can be designed to implement the systems and methods based on the description herein.

When implemented in software, the functions may be stored as one or more instructions or code on a non-transitory computer-readable or processor-readable storage medium. The operations of a method or algorithm disclosed herein may be embodied in a processor-executable software module, which may reside on a computer-readable or processor-readable storage medium. A non-transitory computer-readable or processor-readable media includes both computer storage media and tangible storage media that facilitate transfer of a computer program from one place to another. A non-transitory processor-readable storage media may be any available media that may be accessed by a computer. By way of example, and not limitation, such non-transitory processor-readable media may comprise RAM, ROM, EEPROM, CD-ROM or other optical disk storage, magnetic disk storage or other magnetic storage devices, or any other tangible storage medium that may be used to store desired program code in the form of instructions or data structures and that may be accessed by a computer or processor. Disk and disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and/or instructions on a non-transitory processor-readable medium and/or computer-readable medium, which may be incorporated into a computer program product.

Following is a list of some embodiments:

1. A method comprising:

receiving, by an analytic server, a request for tracing data associated with a first identifier of a first observer device;

generating, by the analytic server responsive to receiving the request, tracing data including a second identifier of a second observer device and a risk score indicative of a degree of association between the first observer device and the second observer device, wherein the degree of association corresponds to at least one of a signal strength of the second observer device, a duration during which the first observer device detects an output from the second observer device having a signal strength at or above a predetermined value, a count of the number of times in which the first observer device detects the output from the second observer device; and transmitting, by the analytic server, the tracing data to a human resource (HR) associated with an organization, wherein the tracing data causes the HR server to notify the second observer device using the second identifier.

2. A system comprising one or more processors configured to cause the system to:

receive, by an analytic server, a request for tracing data associated with a first identifier of a first observer device;

generate, by the analytic server responsive to receiving the request, tracing data including a second identifier of a second observer device and a risk score indicative of a degree of association between the first observer device and the second observer device, wherein the degree of association corresponds to at least one of a signal strength of the second observer device, a duration during which the first observer device detects an output from the second observer device having a signal strength at or above a predetermined value, a count of the number of times in which the first observer device detects the output from the second observer device; and

transmit, by the analytic server, the tracing data to a human resource (HR) associated with an organization, wherein the tracing data causes the HR server to notify the second observer device using the second identifier.

3. A non-transitory computer-readable storage medium storing instructions configured to be executed by one or more processors to cause a system to:

receive, by an analytic server, a request for tracing data associated with a first identifier of a first observer device;

generate, by the analytic server responsive to receiving the request, tracing data including a second identifier of a second observer device and a risk score indicative of a degree of association between the first observer device and the second observer device, wherein the degree of association corresponds to at least one of a signal strength of the second observer device, a duration during which the first observer device detects an output from the second observer device having a signal strength at or above a predetermined value, a count of the number of times in which the first observer device detects the output from the second observer device; and

transmit, by the analytic server, the tracing data to a human resource (HR) associated with an organization, wherein the tracing data causes the HR server to notify the second observer device using the second identifier.

4. A system comprising:

an analytic server configured to:
receive observation data from an observer device, wherein the observer device generates the observation data based on a plurality of wireless signals emitted from a plurality of observer devices;
maintain, in a database and based on the observation data, an association between a plurality of observer identifiers of the plurality of observer devices and a plurality of signal strengths determined by the observer device, each signal strength of the plurality of signal strengths corresponding to a respective wireless signal of the plurality of wireless signals that is emitted by a respective observer device of the plurality of observer devices; and
generate, by the analytic server and based on the database, a risk score indicative of a degree of association between a first observer device and a second observer device.

5. A method comprising:

receiving, by an analytic server, observation data from an observer device, wherein the observer device generates the observation data based on a plurality of wireless signals emitted from a plurality of observer devices;

maintaining, in a database and based on the observation data, an association between a plurality of observer identifiers of the plurality of observer devices and a plurality of signal strengths determined by the observer device, each signal strength of the plurality of signal strengths corresponding to a respective wireless signal of the plurality of wireless signals that is emitted by a respective observer device of the plurality of observer devices; and

generating, by the analytic server and based on the database, a risk score indicative of a degree of association between a first observer device and a second observer device.

6. A non-transitory computer-readable storage medium storing instructions configured to be executed by one or more processors to cause a system to:

receive, by an analytic server, observation data from an observer device, wherein the observer device generates the observation data based on a plurality of wireless signals emitted from a plurality of observer devices;

maintain, in a database and based on the observation data, an association between a plurality of observer identifiers of the plurality of observer devices and a plurality of signal strengths determined by the observer device, each signal strength of the plurality of signal strengths corresponding to a respective wireless signal of the plurality of wireless signals that is emitted by a respective observer device of the plurality of observer devices; and

generate, by the analytic server and based on the database, a risk score indicative of a degree of association between a first observer device and a second observer device.

7. A method comprising:

capturing, by an application executing on a first observer device and responsive to entering a geofence location according to instructions from a human resource (HR) server, a wireless signal emitted from a second observer device;

generating, by the application executing on the first observer device, observation data based on the wireless signal, wherein the observation data indicates a signal strength of the captured wireless signal; and

transmitting, by the application executing on the observer device, the observation data to an analytic server causing the analytic server to determine a risk score that indicates a degree of proximity between the first observer device and the second observer device based on the observation data.

8. A system comprising:

a first observer device configured to, responsive to entering a geofence location according to instructions from a human resource (HR) server:
capture, by an application executing on the first observer device, a wireless signal emitted from a second observer device;
generate, by the application executing on the first observer device, observation data based on the wireless signal, wherein the observation data indicates a signal strength of the captured wireless signal;
transmit, by the application executing on the observer device, the observation data to an analytic server causing the analytic server to determine a risk score that indicates a degree of proximity between the first observer device and the second observer device based on the observation data.

9. A non-transitory computer-readable storage medium storing instructions configured to be executed by one or more processors to cause a system to:

responsive to entering a geofence location according to instructions from a human resource (HR) server:
capture, by an application executing on the first observer device, a wireless signal emitted from a second observer device;
generate, by the application executing on the first observer device, observation data based on the wireless signal, wherein the observation data indicates a signal strength of the captured wireless signal;
transmit, by the application executing on the observer device, the observation data to an analytic server causing the analytic server to determine a risk score that indicates a degree of proximity between the first observer device and the second observer device based on the observation data.

10. A system comprising:

an observation database configured to store user identifiers associated with observation device identifiers; and

a human resource (HR) server configured to:
transmit, to an analytic server, a request for tracing data associated with a first observer device;
receive, from the analytic server, the tracing data, wherein the tracing data includes a risk score indicative of a degree of association between the first observer device and a second observer device;
determine that the risk score exceeds a predetermined threshold; and
transmit, to the second observer device and responsive to determining that the risk score exceeds the predetermined threshold, a message indicating a health status of a user of the second observer device.

11. A non-transitory computer-readable storage medium storing instructions configured to be executed by one or more processors of a system comprising an observation database configured to store user identifiers associated with observation device identifiers and comprising a human resource (HR) serve, wherein the instructions are configured to cause the system to:

transmit, to an analytic server, a request for tracing data associated with a first observer device;

receive, from the analytic server, the tracing data, wherein the tracing data includes a risk score indicative of a degree of association between the first observer device and a second observer device;

determine that the risk score exceeds a predetermined threshold; and

transmit, to the second observer device and responsive to determining that the risk score exceeds the predetermined threshold, a message indicating a health status of a user of the second observer device.

12. A method comprising:

at a system comprising an observation database configured to store user identifiers associated with observation device identifiers and comprising a human resource (HR) server:

transmit, by the HR server, to an analytic server, a request for tracing data associated with a first observer device;

receive, by the HR server, from the analytic server, the tracing data, wherein the tracing data includes a risk score indicative of a degree of association between the first observer device and a second observer device;

determine, by the HR server, that the risk score exceeds a predetermined threshold; and

transmit, by the HR server, to the second observer device and responsive to determining that the risk score exceeds the predetermined threshold, a message indicating a health status of a user of the second observer device.

13. A method comprising:

selecting, by a server, a target device associated with a user;

obtaining, by the server, detection data of the target device;

determining, by the server, a hypercluster associated the target device;

determining, by the server, a pattern of movements of the target device; and

generating, by the server, a risk level data indicating a risk level of the target device, according to the detection data, the hypercluster, and the pattern.

14. The method of embodiment 13, wherein generating the risk level data indicating the risk level of the target device, according to the detection data, the hypercluster, and the pattern includes:

determining, by the server, a first risk score of the target device according to the detection data,

determining, by the server, a second risk score of the hypercluster associated with the target device,

determining, by the server, a third risk score of the target device according to the pattern of movements of the target device,

determining an aggregated risk score, according to the first risk score, the second risk score, and the third risk score, and

determining the risk level of the target device according to the aggregated risk score.

15. The method of embodiment 14, wherein determining, by the server, the first risk score of the target device according to the detection data includes:

comparing, by the server, a number of detections of the target device by a plurality of observer devices against predetermined threshold values, and

determining the first risk score, according to the comparison.

16. The method of embodiment 14, wherein determining, by the server, the second risk score of the hypercluster associated with the target device includes:

aggregating, by the server, risk scores of a plurality of devices associated with the hypercluster, and

determining, by the server, the second risk score of the hypercluster, according to the aggregated risk scores.

17. The method of embodiment 14, wherein determining, by the server, the third risk score of the target device according to the pattern of movements of the target device includes:

determining, by the server, the pattern of movements of the target device, and

predicting, by the server, coexistences of the target device with one or more devices, according to the pattern of movement.

18. A system comprising one or more processors configured to cause the system to:

select, by a server, a target device associated with a user;

obtain, by the server, detection data of the target device;

determine, by the server, a hypercluster associated the target device;

determine, by the server, a pattern of movements of the target device; and

generate, by the server, a risk level data indicating a risk level of the target device, according to the detection data, the hypercluster, and the pattern.

19. A non-transitory computer-readable storage medium storing instructions configured to be executed by one or more processors of a system to cause the system to:

select, by a server, a target device associated with a user;

obtain, by the server, detection data of the target device;

determine, by the server, a hypercluster associated the target device;

determine, by the server, a pattern of movements of the target device; and

generate, by the server, a risk level data indicating a risk level of the target device, according to the detection data, the hypercluster, and the pattern.

20. A method comprising:

selecting, by a server, a target device associated with a user;

determining, by the server, a first hypercluster associated with the target device;

determining, by the server, a coexistence of the first hypercluster with a second hypercluster; and

generating, by the server, a risk level data indicating a risk level of the target device according to the determined coexistence.

21. The method of embodiment 20, wherein generating, by the server, the risk level data indicating the risk level of the target device according to the determined coexistence includes:

determining, by the server, coexistences of one or more devices of the first hypercluster with one or more devices of the second hypercluster; and

determining, by the server, a risk level of the first hypercluster, according to determined coexistences.

22. A system comprising one or more processors configured to cause the system to:

select, by a server, a target device associated with a user;

determine, by the server, a first hypercluster associated with the target device;

determine, by the server, a coexistence of the first hypercluster with a second hypercluster; and

generate, by the server, a risk level data indicating a risk level of the target device according to the determined coexistence.

23. A non-transitory computer-readable storage medium storing instructions configured to be executed by one or more processors to cause a system to:

select, by a server, a target device associated with a user;

determine, by the server, a first hypercluster associated with the target device;

determine, by the server, a coexistence of the first hypercluster with a second hypercluster; and

generate, by the server, a risk level data indicating a risk level of the target device according to the determined coexistence.

24. A method comprising:

selecting, by a server, a target device associated with a user;

determining, by the server, time points of coexistences by the target device with one or more hyperclusters; and

generating, by the server, a risk level data indicating a risk level of the target device according to the time points of coexistences.

25. The method of embodiment 24, wherein generating, by the server, the risk level data indicating the risk level of the target device according to the time points of coexistences includes:

determining, by the server, a first coexistence of the target device with one or more first devices associated with a first hypercluster at a first time point;

determining, by the server, a second coexistence of the target device with one or more second devices associated with a second hypercluster at a second time point; and

determining, by the server, the risk level of the target device according to the first coexistence with the one or more first devices at the first time point and the second coexistence with the one or more second devices at the second device.

26. A system comprising one or more processors configured to cause the system to:

select, by a server, a target device associated with a user;

determine, by the server, time points of coexistences by the target device with one or more hyperclusters; and

generate, by the server, a risk level data indicating a risk level of the target device according to the time points of coexistences.

27. A non-transitory computer-readable storage medium storing instructions configured to be executed by one or more processors to cause a system to:

select, by a server, a target device associated with a user;

determine, by the server, time points of coexistences by the target device with one or more hyperclusters; and

generate, by the server, a risk level data indicating a risk level of the target device according to the time points of coexistences.

28. A method comprising:

receiving, by a server, observation data of a plurality of wireless signals observed by a plurality of observer devices, the observation data comprising spatial proximity and temporal persistence data between the plurality of observer devices;

determining, by the server, that an observer device of the plurality of observer devices was proximate to a proximal grouping of electronic devices at a time point based on observation data associated with the observer device;

identifying, by the server in a database, a label associated with the proximal grouping, the label associated with a weight;

determining, by the server, a risk score for the observer device based on a set of one or more risk factors of the observation data and the weight associated with the proximal group identified by the label; and

transmitting, by the server, a signal comprising the risk score to an administrator client device.

29. The method of embodiment 28, further comprising:

determining, by the server, that the observer device of the plurality of observer devices was proximate to a third proximal grouping of electronic devices at a third time point based on observation data associated with the observer device;

determining, by the server, that the third proximal grouping is not associated with a label; and

responsive to determining that the third proximal grouping is not associated with a label, discarding observation data associated with the third proximal grouping of electronic devices.

30. The method of embodiment 29, wherein determining the risk score for the observer device comprises aggregating the first weight and the second weight.

31. The method of embodiment 29, wherein the first label and the second label each correspond to a location within a building.

32. A method comprising:

receiving, by a server, observation data of a plurality of wireless signals observed by a plurality of observer devices, the observation data comprising spatial proximity and temporal persistence data between the plurality of observer devices;

receiving, by the server, a request to determine a risk score for an observer device for a time period;

identifying, by the server, a proximal grouping of electronic devices with which the observer device has a proximate association during the time period based on the observation data;

identifying, by the server, a label associated with the identified proximal grouping of electronic devices and a weight associated with the label;

determining, by the server, a number of proximate associations between the proximal grouping of electronic devices and the plurality of observer devices within the time period;

adjusting, by the server, the weight associated with the label based on the determined number of proximate associations;

determining, by the server, a risk score for the observer device based on at least the adjusted weight; and

transmitting, by the server, a signal comprising the risk score to a client device.

33. The computer-implemented method of embodiment 32, wherein determining the risk score for the observer device comprises aggregating the first weight and the second weight.

34. The computer-implemented method of embodiment 32, wherein the first label and the second label each correspond to a location within a building.

35. A system comprising one or more processors configured to cause the system to:

receive, by a server, observation data of a plurality of wireless signals observed by a plurality of observer devices, the observation data comprising spatial proximity and temporal persistence data between the plurality of observer devices;

receive, by the server, a request to determine a risk score for an observer device for a time period;

identify, by the server, a proximal grouping of electronic devices with which the observer device has a proximate association during the time period based on the observation data;

identify, by the server, a label associated with the identified proximal grouping of electronic devices and a weight associated with the label;

determine, by the server, a number of proximate associations between the proximal grouping of electronic devices and the plurality of observer devices within the time period;

adjust, by the server, the weight associated with the label based on the determined number of proximate associations;

determine, by the server, a risk score for the observer device based on at least the adjusted weight; and

transmit, by the server, a signal comprising the risk score to a client device.

36. A non-transitory computer-readable storage medium storing instructions configured to be executed by one or more processors to cause a system to:

receive, by a server, observation data of a plurality of wireless signals observed by a plurality of observer devices, the observation data comprising spatial proximity and temporal persistence data between the plurality of observer devices;

receive, by the server, a request to determine a risk score for an observer device for a time period;

identify, by the server, a proximal grouping of electronic devices with which the observer device has a proximate association during the time period based on the observation data;

identify, by the server, a label associated with the identified proximal grouping of electronic devices and a weight associated with the label;

determine, by the server, a number of proximate associations between the proximal grouping of electronic devices and the plurality of observer devices within the time period;

adjust, by the server, the weight associated with the label based on the determined number of proximate associations;

determine, by the server, a risk score for the observer device based on at least the adjusted weight; and

transmit, by the server, a signal comprising the risk score to a client device.

37. A method comprising:

receiving, by a server, observation data of a plurality of wireless signals observed by a plurality of observer devices, the observation data comprising spatial proximity and temporal persistence data between the plurality of observer devices;

determining, by the server, a pattern of an observer device, the pattern indicating an expected location of the observer device at the time point;

receiving, by the server, a request to determine a risk score for an observer device for a time period;

identifying, by the server, a conflict between the expected location of the pattern and an observed location of the observer device indicated in the observation data at a given time point;

responsive to identifying the conflict:
determining, by the server, a risk score for the observer device based on at least a second weight associated with the expected location at the time point; and

transmitting, by the server to a client device, a signal indicating a risk tier for the observer device within the time period.

38. The method of embodiment 37, wherein determining the risk score for the observer device comprises excluding the observation data associated with the observed location at the time point.

39. The method of embodiment 37, wherein the conflict is associated with a second time period in which the observer device is not associated with any observation data.

40. A system comprising one or more processors configured to cause the system to:

receive, by a server, observation data of a plurality of wireless signals observed by a plurality of observer devices, the observation data comprising spatial proximity and temporal persistence data between the plurality of observer devices;

determine, by the server, a pattern of an observer device, the pattern indicating an expected location of the observer device at the time point;

receive, by the server, a request to determine a risk score for an observer device for a time period;

identify, by the server, a conflict between the expected location of the pattern and an observed location of the observer device indicated in the observation data at a given time point;

responsive to identifying the conflict:
determine, by the server, a risk score for the observer device based on at least a second weight associated with the expected location at the time point; and

transmit, by the server to a client device, a signal indicating a risk tier for the observer device within the time period.

41. A non-transitory computer-readable storage medium storing instructions configured to be executed by one or more processors to cause a system to:

receive, by a server, observation data of a plurality of wireless signals observed by a plurality of observer devices, the observation data comprising spatial proximity and temporal persistence data between the plurality of observer devices;

determine, by the server, a pattern of an observer device, the pattern indicating an expected location of the observer device at the time point;

receive, by the server, a request to determine a risk score for an observer device for a time period;

identify, by the server, a conflict between the expected location of the pattern and an observed location of the observer device indicated in the observation data at a given time point;

responsive to identifying the conflict:
determine, by the server, a risk score for the observer device based on at least a second weight associated with the expected location at the time point; and

transmit, by the server to a client device, a signal indicating a risk tier for the observer device within the time period.

42. A method comprising:

selecting, by a server, a target device associated with a user;

receiving, by the server from a plurality of devices, detection data of the target device;

generating, by the server, a profile of the target device based on the detection data from the plurality of devices; and

generating, by the server, a risk level data indicating a risk level of the target device according to the profile of the target device.

43. The method of embodiment 42, wherein generating, by the server, the profile of the target device based on the detection data from the plurality of devices includes:

generating, by the server, a trajectory of locations of the target device.

44. The method of embodiment 42, wherein the target device is a beacon device transmitting a wireless signal, wherein the plurality of devices generate the detection data, in response to detecting the wireless signal from the beacon device.

45. The method of embodiment 42, further comprising:

generating, by the server for another device of the plurality of devices, another risk level data indicating another risk level of the another device according to the risk level of the target device.

46. A system comprising one or more processors configured to cause the system to:

select, by a server, a target device associated with a user;

receive, by the server from a plurality of devices, detection data of the target device;

generate, by the server, a profile of the target device based on the detection data from the plurality of devices; and

generate, by the server, a risk level data indicating a risk level of the target device according to the profile of the target device.

47. A non-transitory computer-readable storage medium storing instructions configured to be executed by one or more processors to cause a system to:

select, by a server, a target device associated with a user;

receive, by the server from a plurality of devices, detection data of the target device;

generate, by the server, a profile of the target device based on the detection data from the plurality of devices; and

generate, by the server, a risk level data indicating a risk level of the target device according to the profile of the target device

48. A system comprising:

a plurality of observer devices configured to generate observation data based on a plurality of wireless signals emitted from each of the plurality of observer devices, wherein the observer devices includes at least one mobile device paired with at least one tracer device respectively, and wherein the observer devices are configured to generate the observation data based on the wireless signals emitted from each of the at least one tracer device;

a mobile device paired with a tracer device, the mobile device configured to receive the wireless signals emitted from the observer devices and transmit the observation data to an analytic server via one or more networks, and the tracer device configured to emit wireless signals containing the observation data for the mobile device paired with the tracer device;

an analytic server configured to:
receive the observation data from each observer device;
identify a proximity relationship between the mobile device and a set of one or more observer devices based up on the observation data received for the mobile device and the set of one or more observer devices; and
generate a risk score for the mobile device based upon the proximity relationship, the risk score indicative of a degree of association between the mobile device each observer device of the set of observer devices.

49. The system according to embodiment 48, wherein each observer device is further configured to determine a signal strength for each of the wireless signals, and wherein the proximity relationship is identified based upon the signal strength of each of the wireless signals.

50. The system according to embodiment 48, wherein the observer device is configured to begin detecting transmitting observation data to the analytic server in response to determining that the observer device is receiving the wireless signals from a threshold number of other observer devices.

51. A non-transitory computer-readable storage medium storing instructions configured to be executed by one or more processors of a system comprising (i) a plurality of observer devices configured to generate observation data based on a plurality of wireless signals emitted from each of the plurality of observer devices, wherein the observer devices includes at least one mobile device paired with at least one tracer device respectively, and wherein the observer devices are configured to generate the observation data based on the wireless signals emitted from each of the at least one tracer device; comprising (ii) a mobile device paired with a tracer device, the mobile device configured to receive the wireless signals emitted from the observer devices and transmit the observation data to an analytic server via one or more networks, and the tracer device configured to emit wireless signals containing the observation data for the mobile device paired with the tracer device; and comprising (iii) an analytic server, wherein the instructions are configured to be executed by the one or more processors of the system to cause the system to:

receive, by the analytic server, the observation data from each observer device;

identify, by the analytic server, a proximity relationship between the mobile device and a set of one or more observer devices based up on the observation data received for the mobile device and the set of one or more observer devices; and

generate, by the analytic server, a risk score for the mobile device based upon the proximity relationship, the risk score indicative of a degree of association between the mobile device each observer device of the set of observer devices.

52. A method comprising:

at a system comprising (i) a plurality of observer devices configured to generate observation data based on a plurality of wireless signals emitted from each of the plurality of observer devices, wherein the observer devices includes at least one mobile device paired with at least one tracer device respectively, and wherein the observer devices are configured to generate the observation data based on the wireless signals emitted from each of the at least one tracer device; (ii) a mobile device paired with a tracer device, the mobile device configured to receive the wireless signals emitted from the observer devices and transmit the observation data to an analytic server via one or more networks, and the tracer device configured to emit wireless signals containing the observation data for the mobile device paired with the tracer device; and (iii) an analytic server configured to:

receive the observation data from each observer device;

identify a proximity relationship between the mobile device and a set of one or more observer devices based up on the observation data received for the mobile device and the set of one or more observer devices; and

generate a risk score for the mobile device based upon the proximity relationship, the risk score indicative of a degree of association between the mobile device each observer device of the set of observer devices.

The preceding description of the disclosed embodiments is provided to enable any person skilled in the art to make or use the embodiments described herein and variations thereof. Various modifications to these embodiments will be readily apparent to those skilled in the art, and the generic principles defined herein may be applied to other embodiments without departing from the spirit or scope of the subject matter disclosed herein. Thus, the present disclosure is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting, with the true scope and spirit being indicated by the following claims.

What is claimed is:

1. A method comprising:
receiving, by an analytic server, a request for tracing data associated with a first identifier of a first observer device;
generating, by the analytic server responsive to receiving the request, tracing data including a second identifier of a second observer device and a risk score indicative of a degree of association between the first observer device and the second observer device, wherein the degree of association corresponds to at least one of a signal strength of the second observer device, a duration during which the first observer device detects an output from the second observer device having a signal strength at or above a predetermined value, a count of the number of times in which the first observer device detects the output from the second observer device; and
transmitting, by the analytic server, the tracing data to a human resource (HR) associated with an organization, wherein the tracing data causes the HR server to notify the second observer device using the second identifier.

2. A system comprising:
an analytic server configured to:
receive observation data from an observer device, wherein the observer device generates the observation data based on a plurality of wireless signals emitted from a plurality of observer devices;
maintain, in a database and based on the observation data, an association between a plurality of observer identifiers of the plurality of observer devices and a plurality of signal strengths determined by the observer device, each signal strength of the plurality of signal strengths corresponding to a respective wireless signal of the plurality of wireless signals that is emitted by a respective observer device of the plurality of observer devices; and
generate, by the analytic server and based on the database, a risk score indicative of a degree of association between a first observer device and a second observer device.

3. A method comprising:
capturing, by an application executing on a first observer device and responsive to entering a geofence location according to instructions from a human resource (HR) server, a wireless signal emitted from a second observer device;
generating, by the application executing on the first observer device, observation data based on the wireless signal, wherein the observation data indicates a signal strength of the captured wireless signal; and
transmitting, by the application executing on the observer device, the observation data to an analytic server causing the analytic server to determine a risk score that indicates a degree of proximity between the first observer device and the second observer device based on the observation data.

4. A system comprising:
a first observer device configured to, responsive to entering a geofence location according to instructions from a human resource (HR) server:
capture, by an application executing on the first observer device, a wireless signal emitted from a second observer device;
generate, by the application executing on the first observer device, observation data based on the wireless signal, wherein the observation data indicates a signal strength of the captured wireless signal;
transmit, by the application executing on the observer device, the observation data to an analytic server causing the analytic server to determine a risk score that indicates a degree of proximity between the first observer device and the second observer device based on the observation data.

5. A system comprising:
an observation database configured to store user identifiers associated with observation device identifiers; and
a human resource (HR) server configured to:
transmit, to an analytic server, a request for tracing data associated with a first observer device;
receive, from the analytic server, the tracing data, wherein the tracing data includes a risk score indicative of a degree of association between the first observer device and a second observer device;
determine that the risk score exceeds a predetermined threshold; and
transmit, to the second observer device and responsive to determining that the risk score exceeds the predetermined threshold, a message indicating a health status of a user of the second observer device.

6. A method comprising:
selecting, by a server, a target device associated with a user;
obtaining, by the server, detection data of the target device;
determining, by the server, a hypercluster associated the target device;
determining, by the server, a pattern of movements of the target device; and
generating, by the server, a risk level data indicating a risk level of the target device, according to the detection data, the hypercluster, and the pattern.

7. The method of claim 6, wherein generating the risk level data indicating the risk level of the target device, according to the detection data, the hypercluster, and the pattern includes:
determining, by the server, a first risk score of the target device according to the detection data,
determining, by the server, a second risk score of the hypercluster associated with the target device,
determining, by the server, a third risk score of the target device according to the pattern of movements of the target device,
determining an aggregated risk score, according to the first risk score, the second risk score,
and the third risk score, and
determining the risk level of the target device according to the aggregated risk score.

8. The method of claim 7, wherein determining, by the server, the first risk score of the target device according to the detection data includes:
comparing, by the server, a number of detections of the target device by a plurality of observer devices against predetermined threshold values, and
determining the first risk score, according to the comparison.

9. The method of claim 7, wherein determining, by the server, the second risk score of the hypercluster associated with the target device includes:
aggregating, by the server, risk scores of a plurality of devices associated with the hypercluster, and
determining, by the server, the second risk score of the hypercluster, according to the aggregated risk scores.

10. The method of claim 7, wherein determining, by the server, the third risk score of the target device according to the pattern of movements of the target device includes:
determining, by the server, the pattern of movements of the target device, and
predicting, by the server, coexistences of the target device with one or more devices,
according to the pattern of movement.

11. A system comprising one or more processors configured to cause the system to:
select, by a server, a target device associated with a user;
obtain, by the server, detection data of the target device;
determine, by the server, a hypercluster associated the target device;
determine, by the server, a pattern of movements of the target device; and
generate, by the server, a risk level data indicating a risk level of the target device, according to the detection data, the hypercluster, and the pattern.
